(19) United States
(12) Patent Application Publication
(10) Pub. No.: US 2011/0183980 A1
(43) Pub. Date: Jul. 28, 2011

(54) O-BENZYL NICOTINAMIDE ANALOGS AS MGLUR5 POSITIVE ALLOSTERIC MODULATORS

(76) Inventors: P. Jeffrey Conn, Brentwood, TN (US); Craig W. Lindsley, Brentwood, TN (US); Charles David Weaver, Franklin, TN (US); Shaun Stauffer, Brentwood, TN (US); Richard Williams, Bangor North Down (IE); Gregor MacDonald, Beerse (BE); Ya Zhou, Nashville, TN (US); José Manuel Bartolomé-Nebreda, Toledo (ES)

(21) Appl. No.: 12/886,950

(22) Filed: Sep. 21, 2010

Related U.S. Application Data
(60) Provisional application No. 61/244,417, filed on Sep. 21, 2009.

(51) Int. Cl. A61K 31/5377; C07D 213/62; C07D 213/78; C07D 401/12; C07D 417/12; C07D 403/12; C07D 413/12; A61K 31/444; A61K 31/44; A61K 31/4427; A61K 31/4439; A61K 31/506; A61P 25/00

(52) U.S. Cl. 514/237.2; 546/261; 546/298; 546/268.1; 546/269.7; 544/333; 544/124; 514/335; 514/350; 514/340; 514/342; 514/256

(57) ABSTRACT

In one aspect, the invention relates to O-benzyl nicotinamide analogs, derivatives thereof, and related compounds, which are useful as positive allosteric modulators of the metabotropic glutamate receptor subtype 5 (mGluR5); synthetic methods for making the compounds; pharmaceutical compositions comprising the compounds; and methods of treating neurological and psychiatric disorders associated with glutamate dysfunction using the compounds and compositions. This abstract is intended as a scanning tool for purposes of searching in the particular art and is not intended to be limiting of the present invention.

### CROSS-REFERENCE TO RELATED APPLICATIONS

This application claims the benefit of U.S. Application No. 61/244,417, filed Sep. 21, 2009, which is hereby incorporated herein by reference in its entirety.

### ACKNOWLEDGMENT

This invention was made with government support under Grant no. 5R01 NS031373-15 awarded by the National Institute of Neurological Disorders and Stroke (NINDS) and Grant no. 5R01MH073676-04 awarded by the National Institute of Mental Health (NIMH). The United States government has certain rights in the invention.

### BACKGROUND

L-glutamic acid, the most commonly occurring neurotransmitter in the central nervous system, plays a role in a large number of physiological processes. The glutamate-dependent stimulus receptors are divided into two main groups. The first main group forms ligand-controlled ion channels. The second main group is metabotropic glutamate receptors (mGluRs), which belong to the family of G-protein-coupled receptors. Metabotropic glutamate receptors, including mGluR5, have been implicated in a wide range of biological functions, indicating a potential role for the mGluR5 receptor in a variety of disease processes in mammals. Ligands of metabotropic glutamate receptors can be used for the treatment or prevention of acute and/or chronic neurological and/or psychiatric disorders associated with glutamate dysfunction, such as psychosis, schizophrenia, age-related cognitive decline, and the like.

Selective positive allosteric modulators are compounds that do not directly activate receptors by themselves, but binding of these compounds increase the affinity of a glutamate-site agonist at its extracellular N-terminal binding site. Positive allosteric modulation (potentiation) is thus an attractive mechanism for enhancing appropriate physiological receptor activation.

Unfortunately, there is a scarcity of selective positive allosteric modulators for the mGluR5 receptor. Further, conventional mGluR5 receptor modulators typically lack satisfactory aqueous solubility and exhibit poor oral bioavailability. Therefore, there remains a need for methods and compositions that overcome these deficiencies and that effectively provide selective positive allosteric modulators for the mGluR5 receptor.

### SUMMARY

In accordance with the purpose(s) of the invention, as embodied and broadly described herein, the invention, in one aspect, relates to compounds useful as positive allosteric modulators (i.e., potentiators) of the metabotropic glutamate receptor subtype 5 (mGluR5), methods of making same, pharmaceutical compositions comprising same, and methods of treating neurological and psychiatric disorders associated with glutamate dysfunction using same.

Disclosed are methods for the treatment of a neurological and/or psychiatric disorder associated with glutamate dysfunction in a mammal comprising the step of administering to the mammal a therapeutically effective amount of least one compound having a structure represented by a formula:

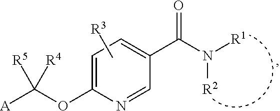

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

Also disclosed are methods for potentiation of metabotropic glutamate receptor activity in a mammal comprising the step of administering to the mammal a therapeutically effective amount of least one compound having a structure represented by a formula:

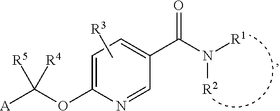

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

Also disclosed are methods for partial agonism of metabotropic glutamate receptor activity in a mammal comprising the step of administering to the mammal a therapeutically effective amount of least one compound having a structure represented by a formula:

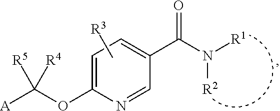

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

Also disclosed are methods for enhancing cognition in a mammal comprising the step of administering to the mammal an effective amount of least one compound having a structure represented by a formula:

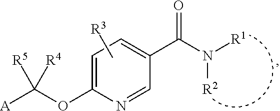

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

Also disclosed are methods for modulating mGluR5 activity in a mammal comprising the step of administering to the mammal an effective amount of least one compound having a structure represented by a formula:

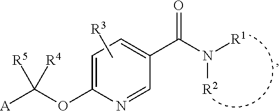

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

Also disclosed are methods for modulating mGluR5 activity in at least one cell, comprising the step of contacting the at least one cell with an effective amount of least one compound having a structure represented by a formula:

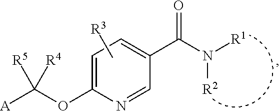

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

Also disclosed are compounds having a structure represented by a formula:

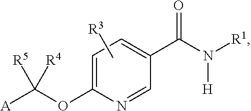

wherein R¹ is an C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide; wherein R³ represents 0-1 substituents independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted C3 to C9 cyclic organic residue selected from aryl, heteroaryl, cycloalkyl, heterocycloalkyl, cycloalkenyl, and heterocycloalkenyl; or a pharmaceutically acceptable salt or N-oxide thereof, wherein the compound exhibits potentiation of mGluR5 response to glutamate as an increase in response to non-maximal concentrations of glutamate in human embryonic kidney cells transfected with rat mGluR5 in the presence of the compound, compared to the response to glutamate in the absence of the compound.

Also disclosed are methods of making a compound, or pharmaceutically acceptable salt or N-oxide thereof, comprising the step of reacting a first compound having a structure represented by a formula:

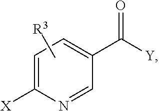

wherein X is halogen; wherein Y is −OR⁶ or —NR¹R²; wherein R⁶ is alkyl or aryl; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; and wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; with a second compound having a structure represented by a formula:

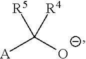

wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and wherein A is an optionally substituted cyclic organic residue selected from aryl, heteroaryl, cycloalkyl, heterocycloalkyl, cycloalkenyl, and heterocycloalkenyl, thereby providing a compound having a structure represented by a formula:

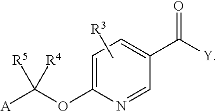

Also disclosed are methods of making a compound, or pharmaceutically acceptable salt or N-oxide thereof, comprising the step of reacting a first compound having a structure represented by a formula:

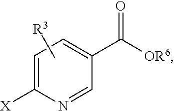

wherein X is halogen or

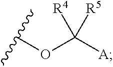

wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl, wherein R⁶ is alkyl or aryl; and wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; with a second compound having a structure represented by a formula:

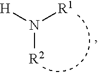

wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons, thereby providing a compound having a structure represented by a formula:

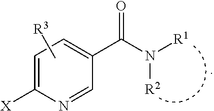

Also disclosed are the products of the disclosed methods.

Also disclosed are methods for manufacturing a medicament comprising combining at least one disclosed compound or at least one disclosed product with a pharmaceutically acceptable carrier or diluent.

Also disclosed are uses of a disclosed compound or a disclosed product in the manufacture of a medicament for the treatment of a disorder associated with glutamate dysfunction in a mammal.

Also disclosed are kits comprising at least one disclosed compound or at least one disclosed product and one or more of at least one agent known to increase mGluR5 activity; at least one agent known to decrease mGluR5 activity; at least one agent known to treat a neurological and/or psychiatric disorder; at least one agent known to treat a disease of uncontrolled cellular proliferation; or instructions for treating a disorder associated with glutamate dysfunction.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a schematic of the NMDA receptor.

FIG. 2 shows a schematic illustrating that activation of mGluR5 potentiates NMDA receptor function.

FIG. 3 illustrates allosteric modulation of mGluR5.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “mGluR5 receptor positive allosteric modulator” refers to any exogenously administered compound or agent that directly or indirectly augments the activity of the mGluR5 receptor in the presence or in the absence of the endogenous ligand (such as glutamate) in an animal, in particular a mammal, for example a human. The term “mGluR5 receptor positive allosteric modulator5 includes a compound that is an “mGluR5 receptor allosteric potentiator” or an “mGluR5 receptor allosteric agonist,” as well as a compound that has mixed activity as both an “mGluR5 receptor allosteric potentiator” and an “mGluR5 receptor allosteric agonist.”

As used herein, the term “mGluR5 receptor allosteric potentiator” refers to any exogenously administered compound or agent that directly or indirectly augments the response produced by the endogenous ligand (such as glutamate) when it binds to the orthosteric site of the mGluR5 receptor in an animal, in particular a mammal, for example a human. The mGluR5 receptor allosteric potentiator binds to a site other than the orthosteric site (an allosteric site) and positively augments the response of the receptor to an agonist. Because it does not induce desensitization of the receptor, activity of a compound as an mGluR5 receptor allosteric potentiator provides advantages over the use of a pure mGluR5 receptor allosteric agonist. Such advantages can include, for example, increased safety margin, higher tolerability, diminished potential for abuse, and reduced toxicity.

As used herein, the term “mGluR5 receptor allosteric agonist” refers to any exogenously administered compound or agent that directly augments the activity of the mGluR5 receptor in the absence of the endogenous ligand (such as glutamate) in an animal, in particular a mammal, for example a human. The mGluR5 receptor allosteric agonist binds to the orthosteric glutamate site of the mGluR5 receptor and directly influences the orthosteric site of the mGluR5 receptor. Because it does not require the presence of the endogenous ligand, activity of a compound as an mGluR5 receptor allosteric agonist provides advantages over the use of a pure mGluR5 receptor allosteric potentiator, such as more rapid onset of action.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more neurological and/or psychiatric disorder associated with glutamate dysfunction prior to the administering step. In some aspects of the disclosed method, the subject has been diagnosed with a need for positive allosteric modulation of metabotropic glutamate receptor activity prior to the administering step. In some aspects of the disclosed method, the subject has been diagnosed with a need for partial agonism of metabotropic glutamate receptor activity prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by modulation of mGluR5” means having been subjected to a physical examination by a person of skill, for example, a physician, and found to have a condition that can be diagnosed or treated by a compound or composition that can modulate mGluR5. As a further example, “diagnosed with a need for modulation of mGluR5” refers to having been subjected to a physical examination by a person of skill, for example, a physician, and found to have a condition characterized by mGluR5 activity. Such a diagnosis can be in reference to a disorder, such as a neurodegenerative disease, and the like, as discussed herein. For example, the term “diagnosed with a need for positive allosteric modulation of metabotropic glutamate receptor activity” refers to having been subjected to a physical examination by a person of skill, for example, a physician, and found to have a condition that can be diagnosed or treated by positive allosteric modulation of metabotropic glutamate receptor activity. For example, “diagnosed with a need for partial antagonism of metabotropic glutamate receptor activity” means having been subjected to a physical examination by a person of skill, for example, a physician, and found to have a condition that can be diagnosed or treated by partial antagonism of metabotropic glutamate receptor activity. For example, “diagnosed with a need for treatment of one or more neurological and/or psychiatric disorder associated with glutamate dysfunction” means having been subjected to a physical examination by a person of skill, for example, a physician, and found to have one or more neurological and/or psychiatric disorder associated with glutamate dysfunction.

In some aspects of the disclosed methods, the subject has been diagnosed with a need for treatment of one or more neurological and/or psychiatric disorder associated with glutamate dysfunction prior to the administering step. In some aspects of the disclosed method, the subject has been diagnosed with a need for potentiation of metabotropic glutamate receptor activity prior to the administering step. In some aspects of the disclosed method, the subject has been diagnosed with a need for partial agonism of metabotropic glutamate receptor activity prior to the administering step.

As used herein, the term “diagnosed with a need for potentiation of metabotropic glutamate receptor activity” refers to having been subjected to a physical examination by a person of skill, for example, a physician, and found to have a condition that can be diagnosed or treated by potentiation of metabotropic glutamate receptor activity. As used herein, “diagnosed with a need for partial agonism of metabotropic glutamate receptor activity” means having been subjected to a physical examination by a person of skill, for example, a physician, and found to have a condition that can be diagnosed or treated by partial agonism of metabotropic glutamate receptor activity. As used herein, “diagnosed with a need for treatment of one or more neurological and/or psychiatric disorder associated with glutamate dysfunction” means having been subjected to a physical examination by a person of skill, for example, a physician, and found to have one or more neurological and/or psychiatric disorder associated with glutamate dysfunction.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to mGluR5 activity) based upon diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target histamine receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “hydrolysable residue” is meant to refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂, where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²—OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —o—(CH₂)₀₋₄C(O)OR^{o}; —(CH₂)₀₋₄—CH(OR^{O})₂; —(CH₂)₀₋₄SR^{o}; —(CH₂)₀₋₄Ph, which may be substituted with R^{o}; —(CH₂)₀₋₄—O—(CH₂)₀₋₁Ph which may be substituted with R^{o}; —CH═CHPh, which may be substituted with R^{o}; —(CH₂)₀₋₄—O—(CH₂)₀₋₁₋pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o})₂; —(CH₂)₀₋₄N(R^{o})C(O)OR^{o}; —N(R^{o})N(R^{o})C(O)R^{o}; —N(R^{o})N(R^{o})C(O)NR^{o})₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o})₂; —C(S)NR^{o})₂; —C(S)SR^{o}; —SC(S)SR^{o}, —(CH₂)₀₋₄OC(O)NR^{o})₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; (CH₂)₀₋₄OS(O)₂R^{o}; —S(O)₂NR^{o})₂; —(CH₂)₀₋₄S(O)R^{o}; —N(R^{o})S(O)₂NR^{o})₂; —N(R^{o})S(O)₂R^{o}; —N(OR^{o})R^{o}; —C(NH)NR^{o})₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—) N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, —(haloR^{•}), —(CH₂)₂₋₀H, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

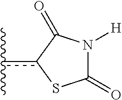

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (ee). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are minor image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non- isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

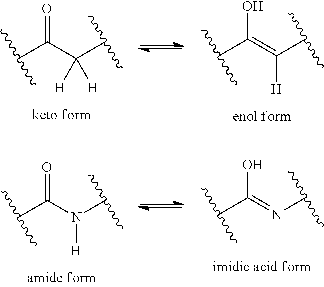

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

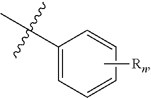

which is understood to be equivalent to a formula:

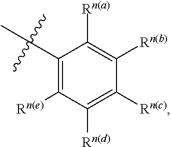

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substitutent. By “independent substituents,” it is meant that each R substituent can be independently defined. For example, if in one instance R^{n(a)} is halogen, then R^{n(b)} is not necessarily halogen in that instance.

In some yet further aspects, a structure of a compound can be represented by a formula:

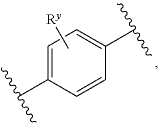

wherein R^{y} represents, for example, 0-2 independent substituents selected from A¹, A², and A³, which is understood to be equivalent to the groups of formulae:
wherein R^{y} represents 0 independent substituents

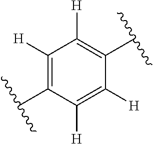

wherein R^{y} represents 1 independent substituent

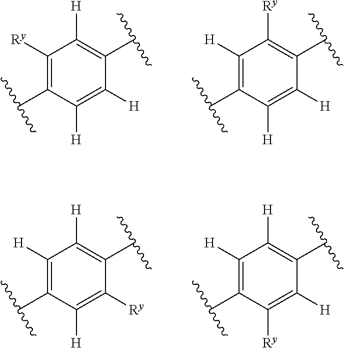

wherein R^{y} represents 2 independent substituents

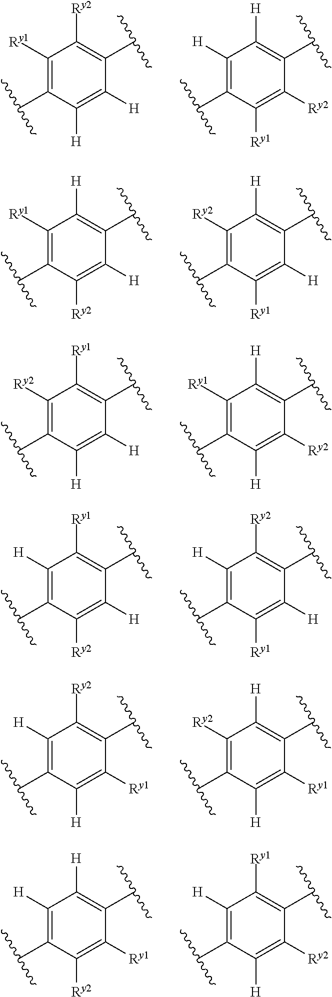

Again, by “independent substituents,” it is meant that each R substituent can be independently defined. For example, if in one instance R^{y1} is A¹, then R^{y2} is not necessarily A¹ in that instance.

In some further aspects, a structure of a compound can be represented by a formula,

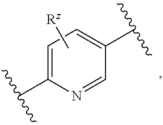

wherein, for example, R^{z} comprises three substituents independently selected from hydrogen and A, which is understood to be equivalent to a formula:

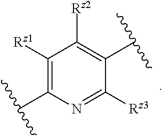

Again, by “independent substituents,” it is meant that each IV substituent is independently defined as hydrogen or A, which is understood to be equivalent to the groups of formulae:
wherein R^{z} comprises three substituents independently selected from H and A

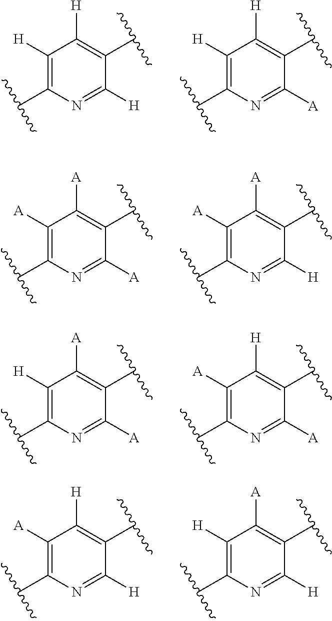

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

B. Development of Novel Allosteric Potentiators of mGluR5

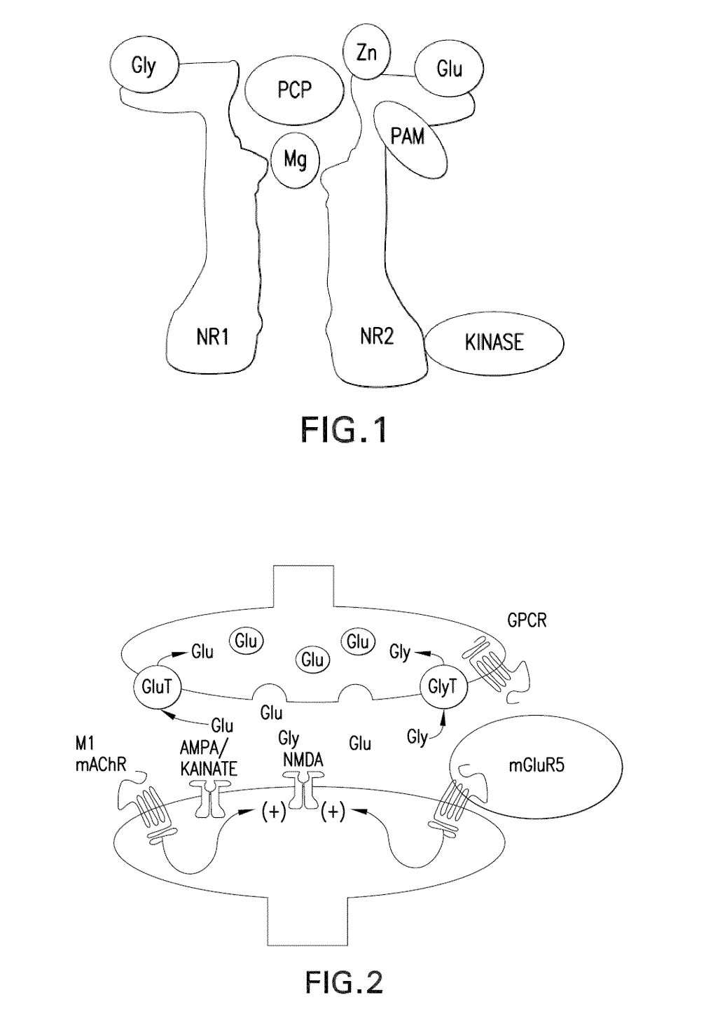

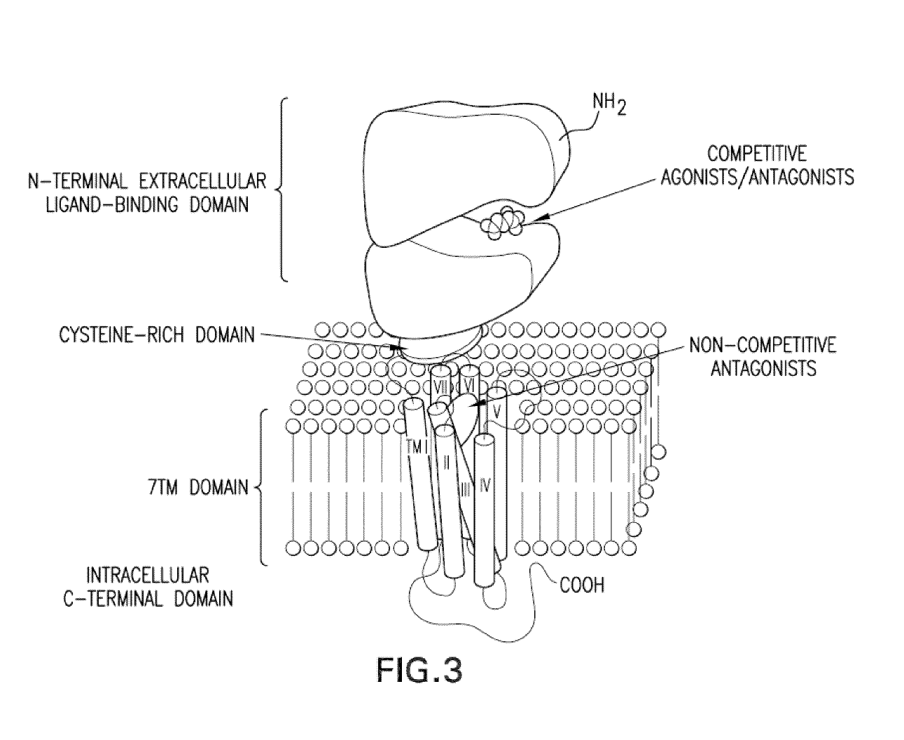

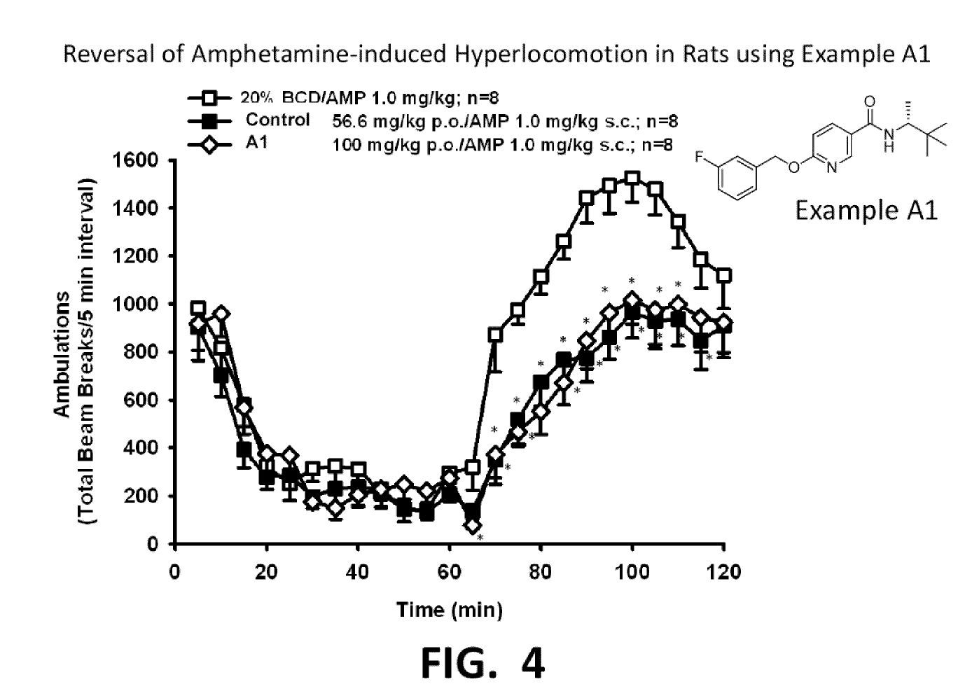

Phencyclidine (PCP) and other NMDA receptor antagonists induce a psychotic state in humans similar to schizophrenia. In schizophrenia patients, PCP and ketamine exacerbate/precipitate preexisting positive and negative symptoms in stable patients. Treatment with NMDA receptor co-agonists can improve positive and negative symptoms. A schematic of the NMDA receptor is shown in FIG. 1. Activation of mGluR5 potentiates NMDA receptor function. See FIG. 2. Orthosteric ligands lack subtype selectivity and can cause unwanted side effects. Allosteric modulators (see FIG. 3) targeting transmembrane domain offer an alternative: TMD is significantly less conserved.

### C. Compounds

In one aspect, the invention relates to compounds useful as positive allosteric modulators (potentiators) of the metabotropic glutamate receptor subtype 5 (mGluR5). More specifically, in one aspect, the present invention relates to compounds that allosterically modulate mGluR5 receptor activity, affecting the sensitivity of mGluR5 receptors to agonists without acting as orthosteric agonists themselves. The compounds of the invention are useful in the treatment neurological and psychiatric disorders associated with glutamate dysfunction and other diseases in which metabotropic glutamate receptors are involved, as further described herein.

1. Structure

In one aspect, the invention relates to a compound has a structure represented by a formula:

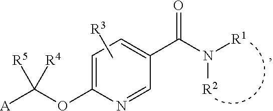

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, R¹ and R² are independently an optionally substituted C1 to C12 organic residue selected from alkyl, aryl, heteroaryl, cycloalkyl, heterocycloalkyl, cycloalkenyl, and heterocycloalkenyl.

In a further aspect, R² is hydrogen and R¹ is an optionally substituted C1 to C12 organic residue selected from alkyl, aryl, heteroaryl, cycloalkyl, heterocycloalkyl, cycloalkenyl, and heterocycloalkenyl.

In a further aspect, N, R¹, and R² together comprise an optionally substituted heterocyclic ring having from two to seven carbons.

In a further aspect, each R¹ and R² is optionally substituted with one or more of halide, hydroxyl, trifluoromethyl, cyano, nitro, amino, alkylamino, dialkylamino, azide, thiol, carboxyl, C1 to C4 alkoxy, C1 to C4 carboxamide, C1 to C4 alkylsulfonyl, or C1 to C4 sulfonamide.

In a further aspect, a compound has a structure represented by a formula:

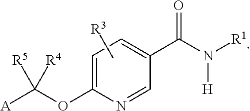

wherein R¹ is an C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide; wherein R³ represents 0-1 substituents independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted C3 to C9 cyclic organic residue selected from aryl, heteroaryl, cycloalkyl, heterocycloalkyl, cycloalkenyl, and heterocycloalkenyl; or a pharmaceutically acceptable salt or N-oxide thereof, wherein the compound exhibits potentiation of mGluR5 response to glutamate as an increase in response to non-maximal concentrations of glutamate in human embryonic kidney cells transfected with rat mGluR5 in the presence of the compound, compared to the response to glutamate in the absence of the compound.

In one aspect, the compound has a structure represented by a formula:

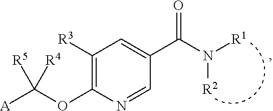

wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

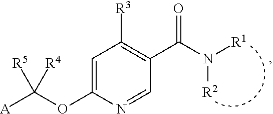

wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

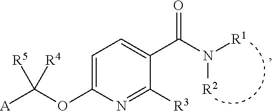

wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

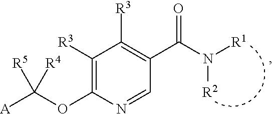

wherein each R³ is independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

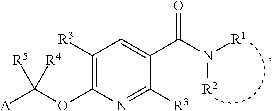

wherein each R³ is independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

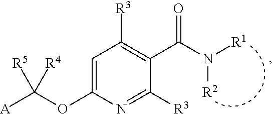

wherein each R³ is independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

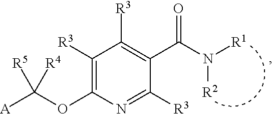

wherein each R³ is independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

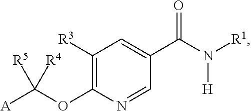

wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

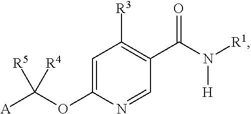

wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

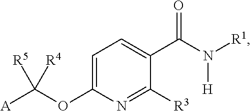

wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, the compound has a structure represented by a formula:

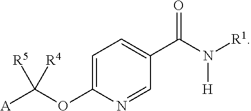

a. R¹Groups

In one aspect, R¹ is an optionally substituted C1 to C12 organic residue selected from alkyl, aryl, heteroaryl, cycloalkyl, heterocycloalkyl, cycloalkenyl, and heterocycloalkenyl. In a further aspect, R¹ is a C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide.

In a further aspect, R¹ is optionally substituted C1 to C9 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, and cyclononyl. In a further aspect, R¹ is optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R¹ is C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl.

In a further aspect, R¹ is optionally substituted aryl selected from phenyl and phenyl substituted with 1-3 groups independently selected from halide, hydroxyl, trifluoromethyl, cyano, nitro, azide, C1 to C4 alkoxy, thiol, C1 to C4 alkylsulfonyl, and C1 to C4 sulfonamide. In a further aspect, R¹ is optionally substituted heteroaryl selected from oxazolyl, isoxazolyl, pyrazolyl, furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl. In a further aspect, R¹ is optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl. In a further aspect, R¹ is optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane. In a further aspect, R¹ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl. In a further aspect, R¹ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹ has a structure represented by a formula:

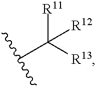

wherein R¹¹≠R¹²≠R¹³ and wherein R¹¹, R¹², and R¹³ are independently selected from hydrogen, an optionally substituted C1 to C12 organic residue selected from alkyl, aryl, heteroaryl, cycloalkyl, heterocycloalkyl, cycloalkenyl, and heterocycloalkenyl, or two of R¹¹, R¹², and R¹³, together with the intermediate carbon, comprise an optionally substituted heterocyclic ring having from two to seven carbons, while the other of R¹¹, R¹², and R¹³ is hydrogen, an optionally substituted C1 to C12 organic residue selected from alkyl, aryl, heteroaryl, cycloalkyl, heterocycloalkyl, cycloalkenyl, or heterocycloalkenyl, thereby forming a stereocenter at the intermediate carbon.

In a further aspect, R¹¹, R¹², and R¹³ are independently selected from hydrogen, an optionally substituted C1 to C12 organic residue selected from alkyl, aryl, heteroaryl, cycloalkyl, heterocycloalkyl, cycloalkenyl, and heterocycloalkenyl. In a further aspect, two of R¹¹, R¹², and R¹³, together with the intermediate carbon, comprise an optionally substituted heterocyclic ring having from two to seven carbons, while the other of R¹¹, R¹² and R¹³ is hydrogen, an optionally substituted C1 to C12 organic residue selected from alkyl, aryl, heteroaryl, cycloalkyl, heterocycloalkyl, cycloalkenyl, or heterocycloalkenyl. In a further aspect, one of R¹¹, R¹², and R¹³ is hydrogen. In a further aspect, none of R¹¹, R¹², and R¹³ is hydrogen. In a further aspect, R¹¹, R¹², and R¹³ are independently selected from hydrogen and C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide.

In one aspect, one enantiomer of the compound has an about three-fold lower EC₅₀ for positive allosteric modulation of mGluR5 than the opposite enantiomer. In a further aspect, one enantiomer of the compound has an about five-fold lower EC₅₀ for positive allosteric modulation of mGluR5 than the opposite enantiomer. In a further aspect, one enantiomer of the compound has an about ten-fold lower EC₅₀ for positive allosteric modulation of mGluR5 than the opposite enantiomer.

In one aspect, the intermediate carbon has a stereochemistry of R. In a further aspect, the compound having a stereochemistry of R at the intermediate carbon has an about three-fold lower EC₅₀ for positive allosteric modulation of mGluR5 than the corresponding S enantiomer. In a further aspect, the compound having a stereochemistry of R at the intermediate carbon has an about five-fold lower EC₅₀ for positive allosteric modulation of mGluR5 than the corresponding S enantiomer. In a further aspect, the compound having a stereochemistry of R at the intermediate carbon has an about ten-fold lower EC₅₀ for positive allosteric modulation of mGluR5 than the corresponding S enantiomer.

In a further aspect, R¹ is selected from:

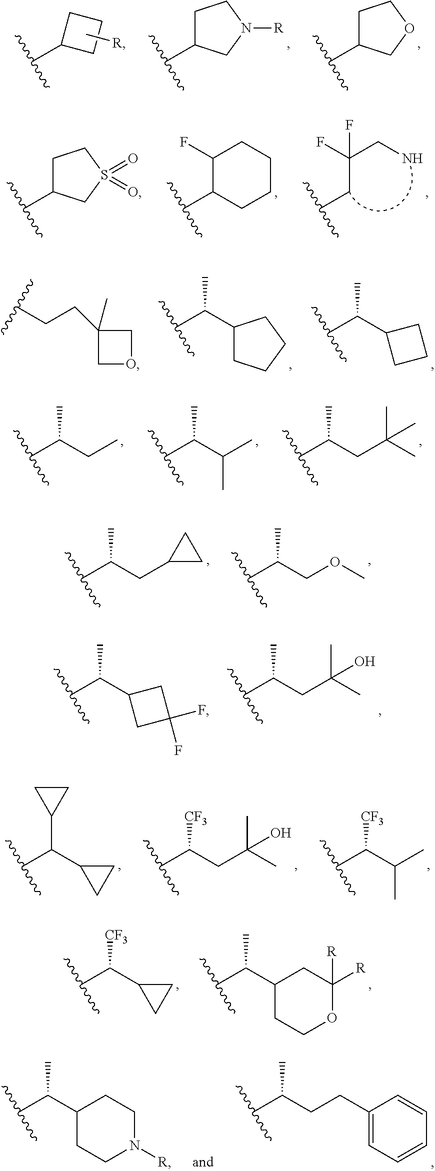

wherein each R is independently selected from hydrogen and C1-C4 alkyl.

b. R² Groups

In one aspect, R² is hydrogen, an optionally substituted C1 to C12 organic residue selected from alkyl, aryl, heteroaryl, cycloalkyl, heterocycloalkyl, cycloalkenyl, and heterocycloalkenyl. In a further aspect, R² is hydrogen. In a further aspect, R² is hydrogen, methyl, ethyl, propyl, or butyl.

c. R³ Groups

In one aspect, R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide. In a further aspect, non-hydrogen R³ is absent.

In a further aspect, R³ is present as one non-hydrogen substituent selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide. In a further aspect, R³ is present as two non-hydrogen substituents selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

In a further aspect, R³ is trifluoromethyl.

d. R⁴ Groups

In one aspect, R⁴ is hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl. In a further aspect, R⁴ is hydrogen. In a further aspect, R⁴ and R⁵ are hydrogen. In a further aspect, R⁴ is C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide.

In a further aspect, R⁴ and R⁵, together with the intermediate carbon, comprise an optionally substituted C3 to C6 cycloalkyl or heterocycloalkyl.

e. R⁵ Groups

In one aspect, R⁵ is hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl. In a further aspect, R⁴ is hydrogen. In a further aspect, R⁴ and R⁵ are hydrogen. In a further aspect, R⁵ is C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide.

In a further aspect, R⁴ and R⁵, together with the intermediate carbon, comprise an optionally substituted C3 to C6 cycloalkyl or heterocycloalkyl.

f. A Groups

In one aspect, A can be an optionally substituted cyclic organic residue selected from aryl, heteroaryl, cycloalkyl, heterocycloalkyl, cycloalkenyl, and heterocycloalkenyl.

In a further aspect, A is optionally substituted aryl selected from phenyl and naphthyl.

In a further aspect, A is optionally substituted heteroaryl selected from oxazolyl, isoxazolyl, pyrazolyl, furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, A is optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, A is optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, A is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, A is optionally substituted heterocycloalkenyl comprising pyrazolinone, imidazolinone, or a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, A is selected from

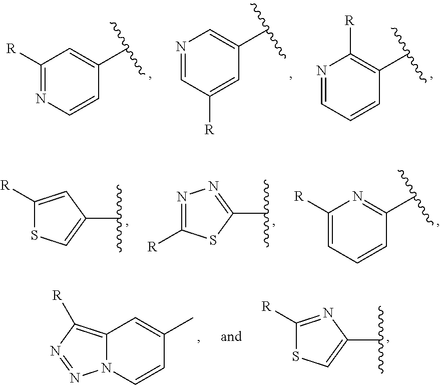

wherein each R is independently selected from hydrogen and C1-C4 alkyl.

2. Activity

Generally, the disclosed compounds exhibit potentiation of mGluR5 response to glutamate as an increase in response to non-maximal concentrations of glutamate in human embryonic kidney cells transfected with rat mGluR5 in the presence of the compound, compared to the response to glutamate in the absence of the compound. For example, a compound can exhibit positive allosteric modulation of mGluR5 with an EC₅₀ of less than about 10,000 nM, of less than about 5,000 nM. of less than about 1,000 nM, of less than about 500 nM, or of less than about 100 nM.

EC₅₀ data for certain exemplified compounds are tabulated in Table 1.

3. Stereoisomer-Dependent Differential mGluR5Activity

In one aspect, one enantiomer of a disclosed compound modulates mGluR5 activity more potently than the opposite enantiomer. For example, a particular enantiomer of a disclosed compound can have an EC₅₀ of less than about 10 μM, of less than about 5 μM, of less than about 1 μM, of less than about 500 nM, of less than about 100 nM, or of less than about 50 nM, while the opposite enantiomer of the disclosed compound has an EC₅₀ of >10 μm.

In a further aspect, the R-enantiomer of a disclosed compound modulates mGluR5 activity more potently than the corresponding S-enantiomer. For example, a particular R-enantiomer of a disclosed compound can have an EC₅₀ of less than about 10 μM, of less than about 5 μM, of less than about 1 μM, of less than about 500 nM, of less than about 100 nM, or of less than about 50 nM, while the corresponding S-enantiomer of the disclosed compound has an EC₅₀ of >10 μM.

In a further aspect, one enantiomer of a disclosed compound modulates mGluR5 activity more potently than the opposite enantiomer. For example, a particular enantiomer of a disclosed compound can have an EC₅₀ of less than about 10%, of less than about 20%, of less than about 30%, of less than about 40%, of less than about 50%, or of less than about 75% of the EC₅₀ of the opposite enantiomer.

In a further aspect, the R-enantiomer of a disclosed compound modulates mGluR5 activity more potently than the corresponding S-enantiomer. For example, a particular R-enantiomer of a disclosed compound can have an EC₅₀ of less than about 10%, of less than about 20%, of less than about 30%, of less than about 40%, of less than about 50%, or of less than about 75% of the EC₅₀ of the corresponding S-enantiomer.

As illustrated below, Example 1.2a.2, (R)-6-(Benzyloxy)-N-(1-cyclohexylethyl)nicotinamide, displays an EC₅₀ of 40 nM and Glu max of 91% against an mGluR5 expressing cell line. In contrast, the opposite stereochemical enantiomer with the S configuration, (S)-6-(benzyloxy)-N-(1-cyclohexylethyl)nicotinamide (prepared in a manner similar to 1.2a.2: LC-MS (214 nm) >98%, 339.2 (M+H)), was found to have an EC₅₀ of >10 μM as an mGluR5 potentiator.

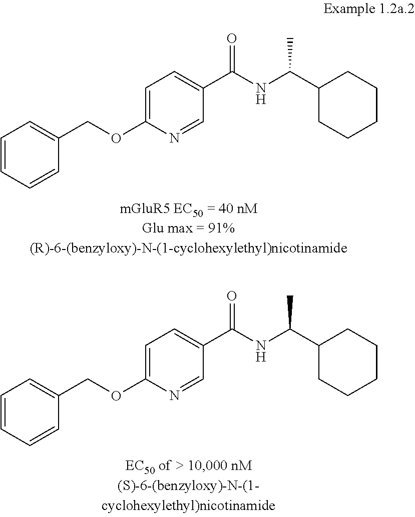

Similarly, as shown below for the 3-fluorophenyl substituted analogs, Example 1.2c.18 and its opposite enantiomer, specificity for potentiation is inherent to Example 1.2c.18 containing the R-configuration for the alpha carbon stereochemistry, while the enantiomer containing the S-configuration has EC₅₀ of >10 μM for modulation of mGluR5 activity.

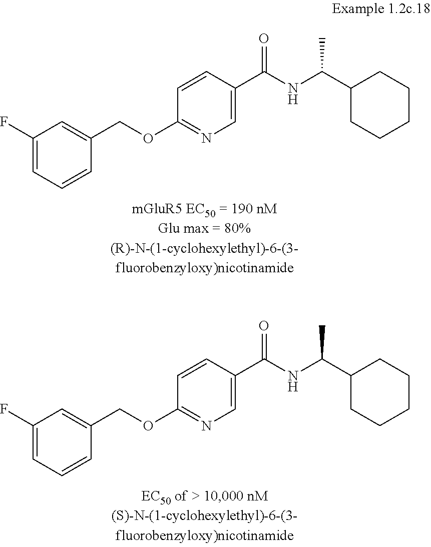

While the disclosed compounds can be provided as a mixture of both the R-enantiomer and the S-enantiomer, it can be desired to provide the mixture of enantiomers of a disclosed compound enriched in the more potent compound. Such can be desired in order to, for example, increase the concentration of an active (or more active) enantiomer or in order to decrease the concentration of a less active (or inactive) enantiomer. Such can improve potency of a pharmaceutical preparation. Such also can minimize undesired side-effects present in a less active enantiomer and not present (or less present) in a more active enantiomer.

Thus, in various aspects, a disclosed compound can be provided in a form enriched in R-enantiomer of the compound. For example, a disclosed compound can be provided in an enantiomeric excess of greater than 50%, greater than 60%, greater than 70%, greater than 75%, greater than 80%, greater than 85%, greater than 90%, greater than 95%, greater than 98%, or greater than 99% of the R-enantiomer of the compound. In one aspect, the R-enantiomer is substantially free from the S-enantiomer. For example, the “R” forms of the compounds can be provided substantially free from the “S” forms of the compounds.

4. Example Compounds

In one aspect, the invention relates to a compound having a structure represented by a structure:

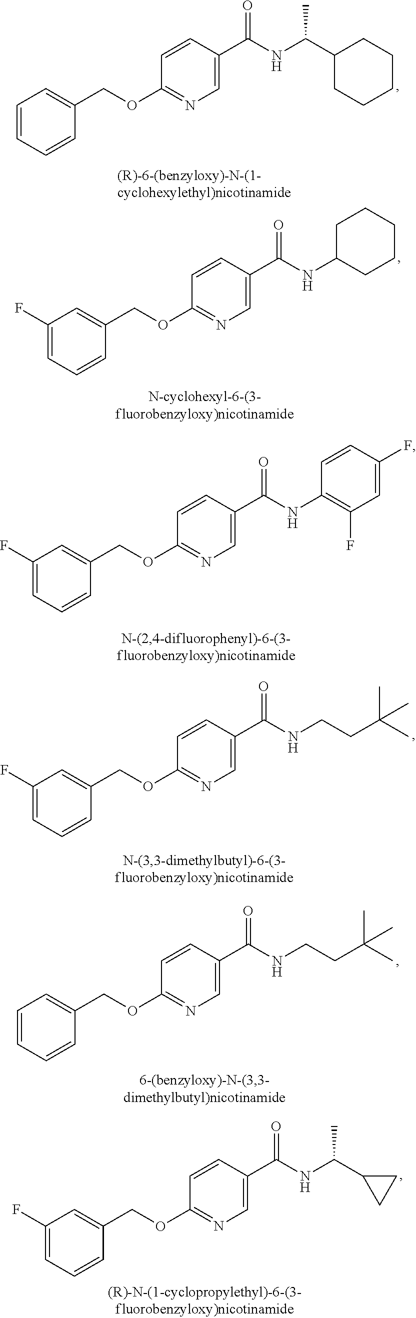

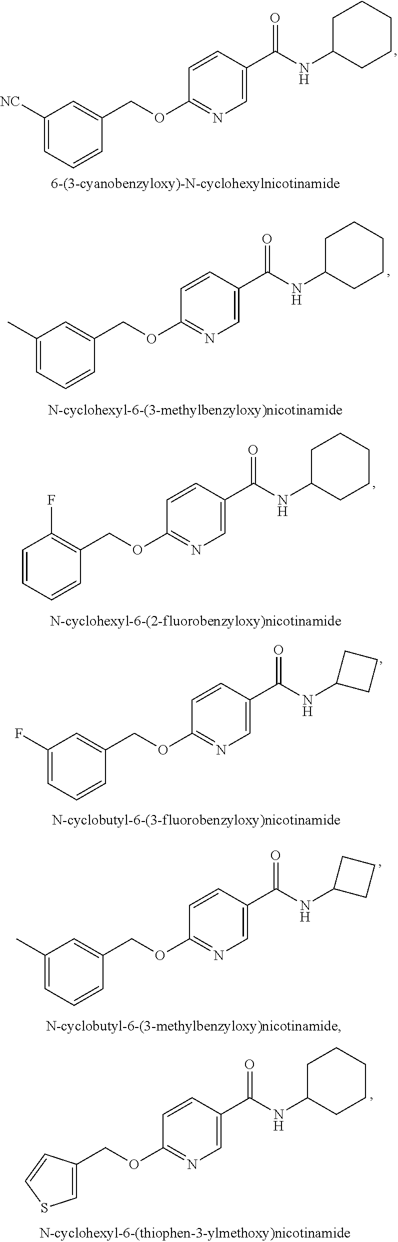

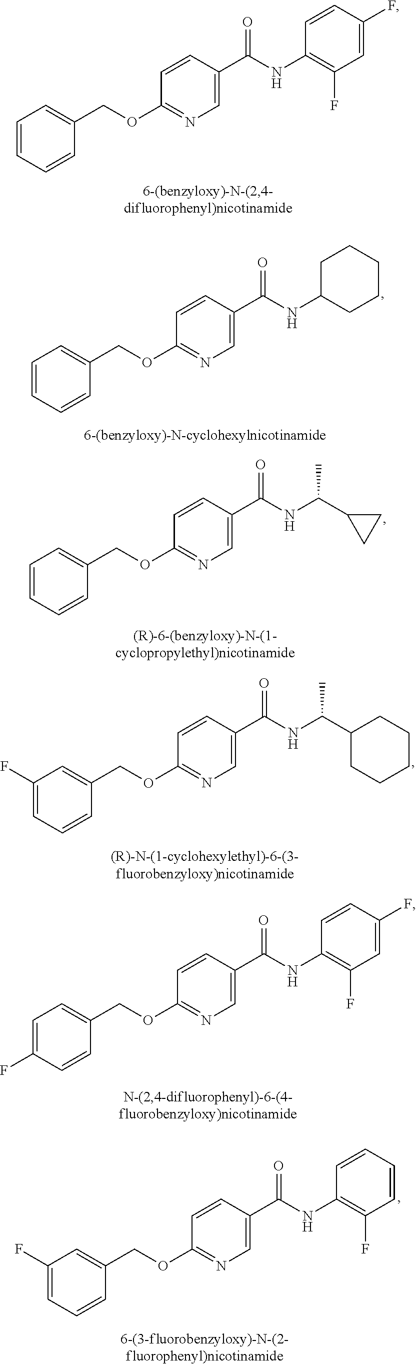

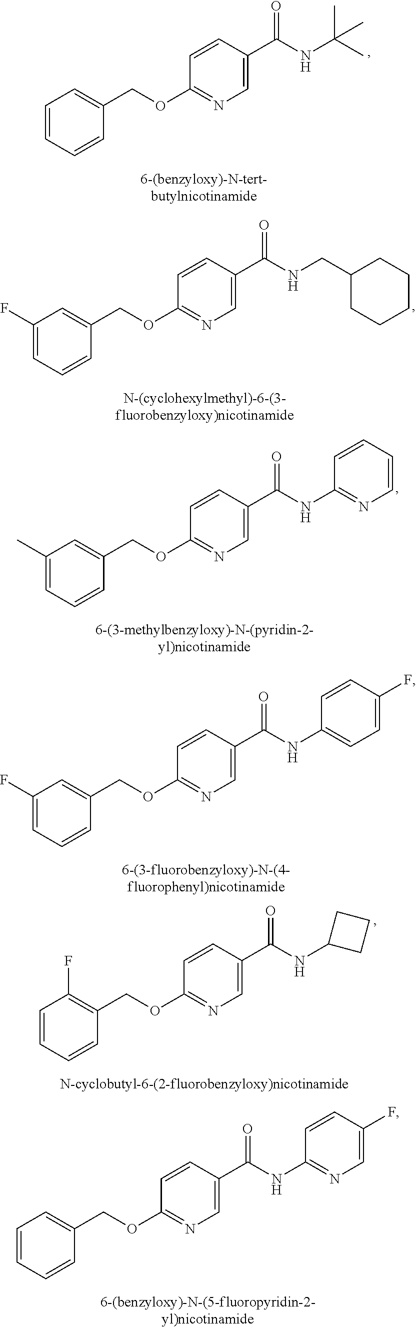

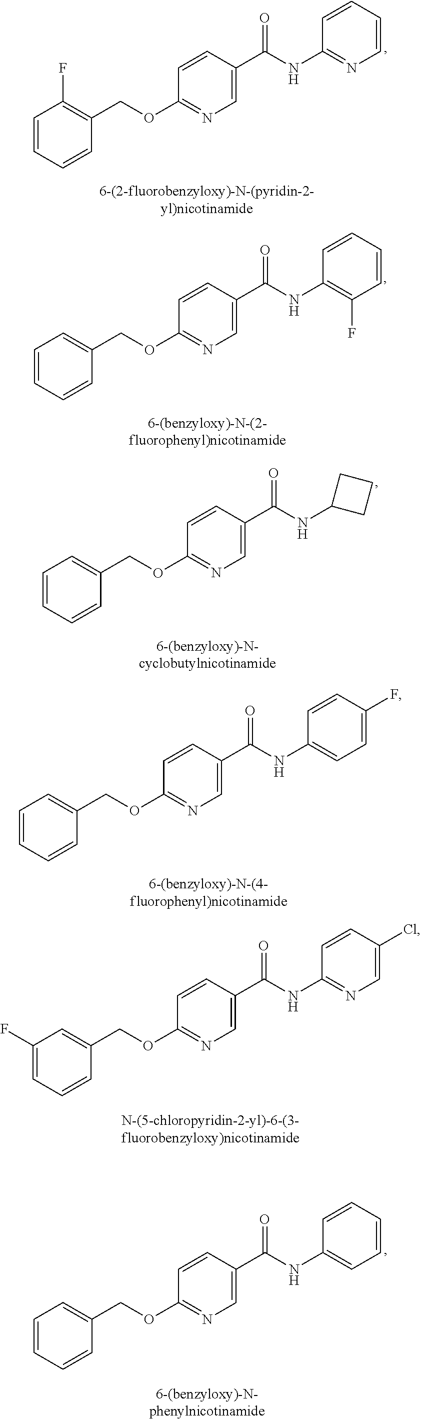

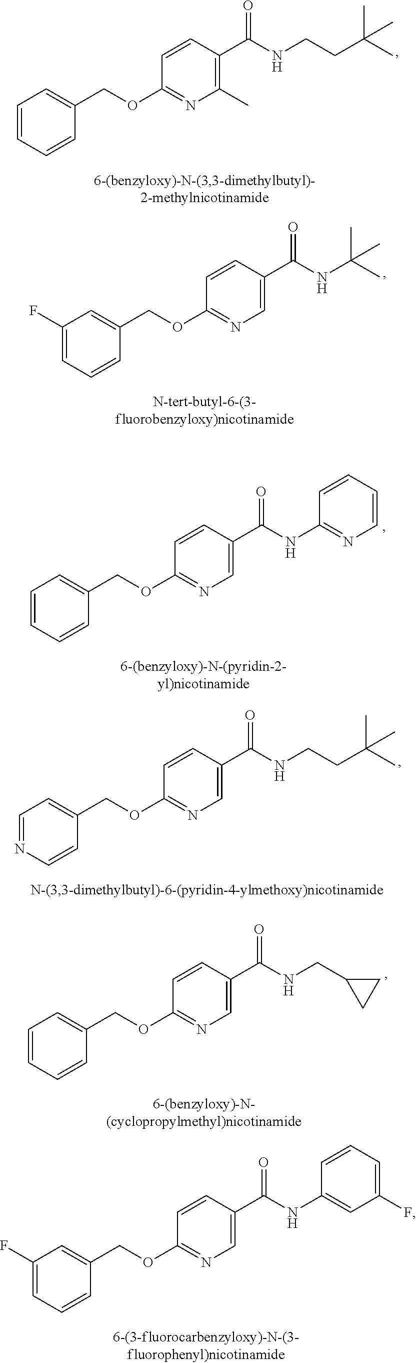

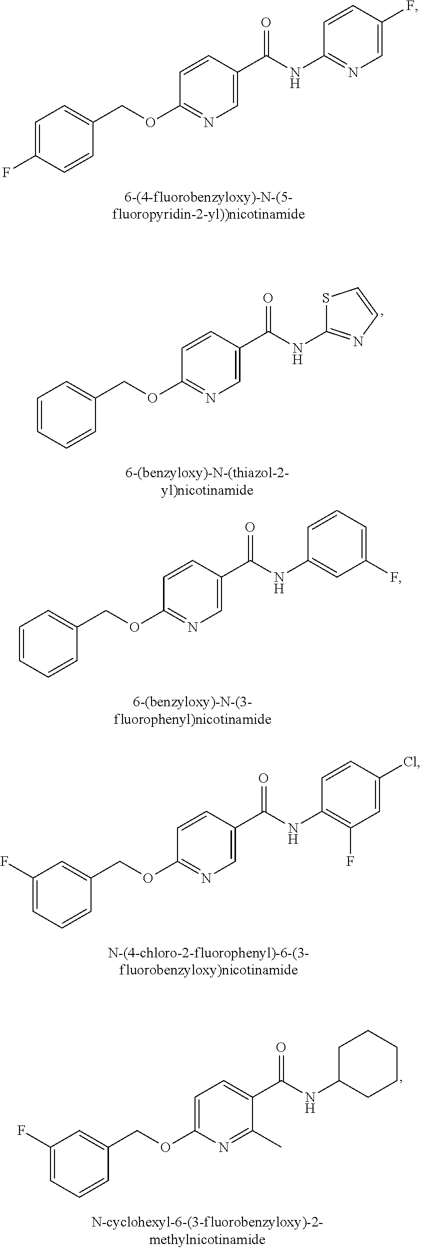

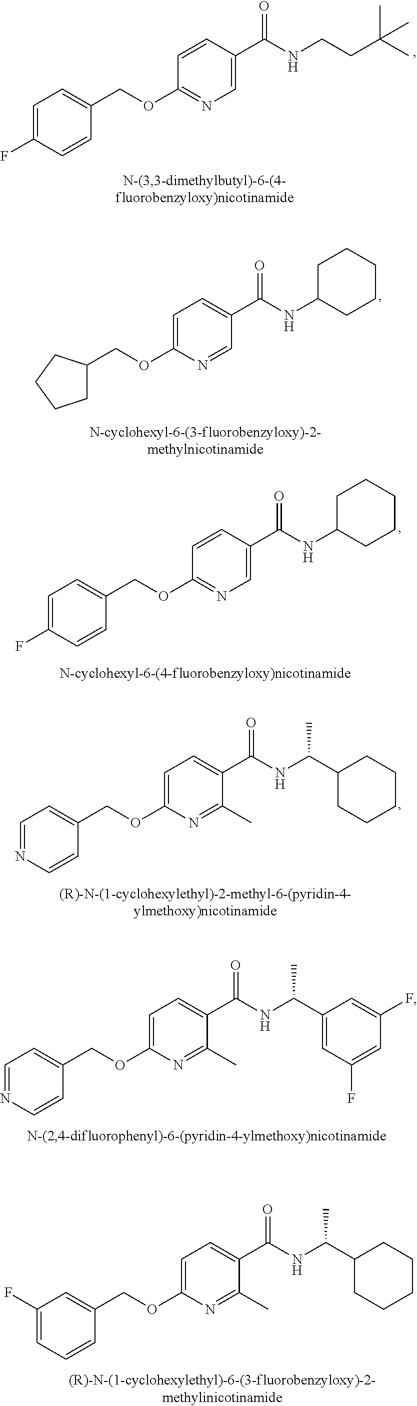

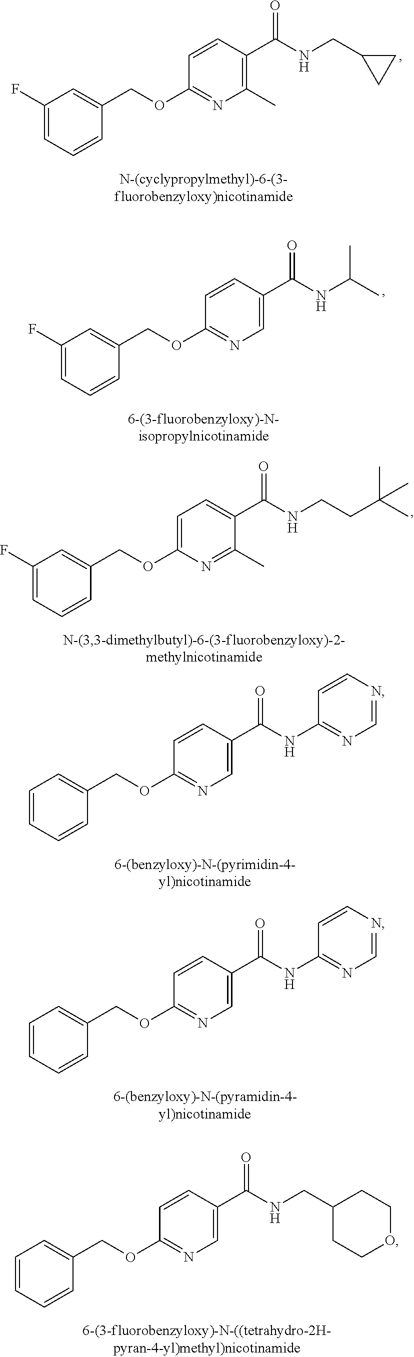

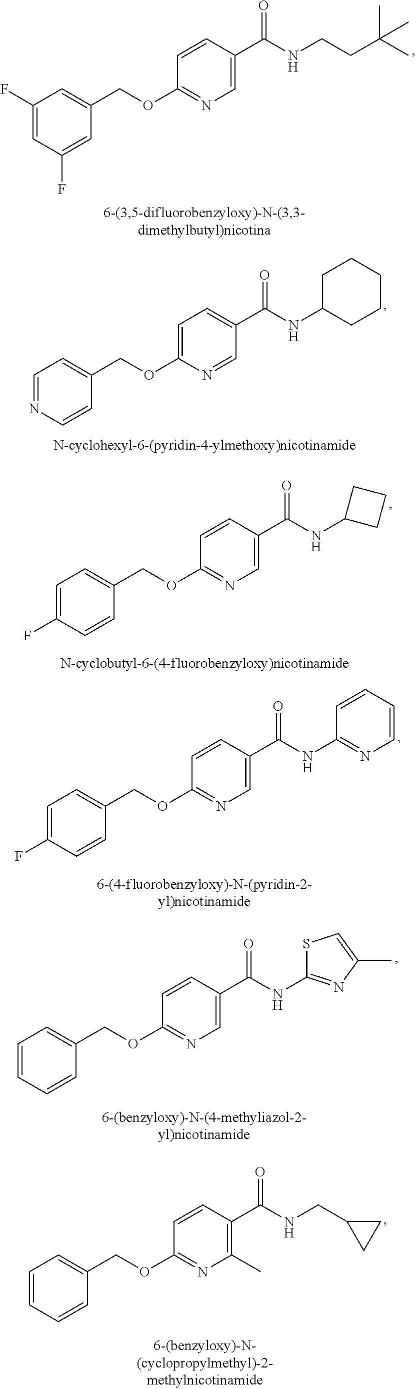

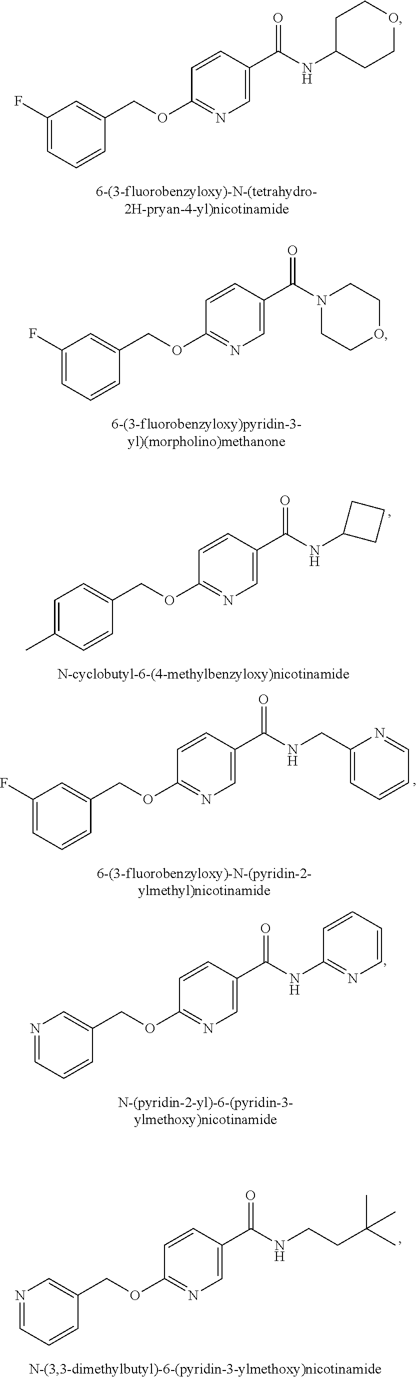

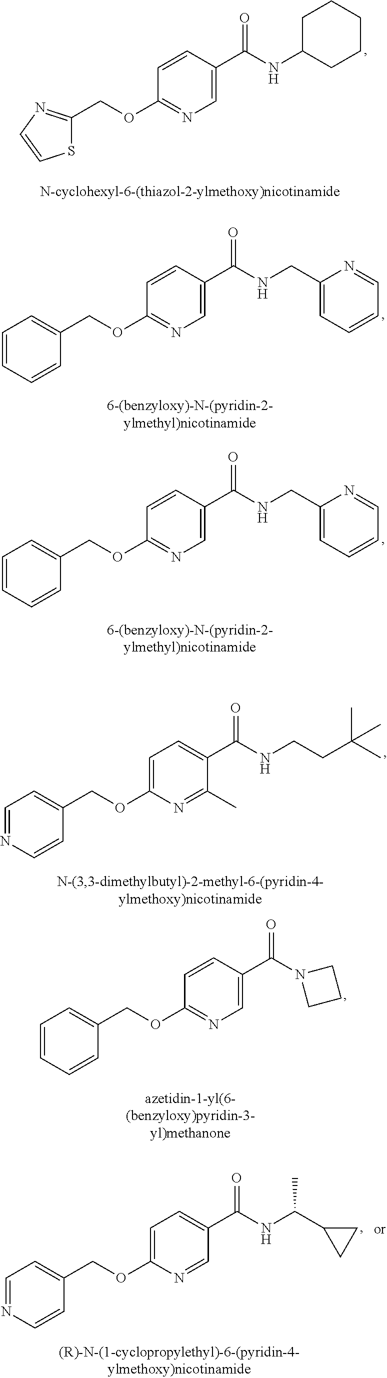

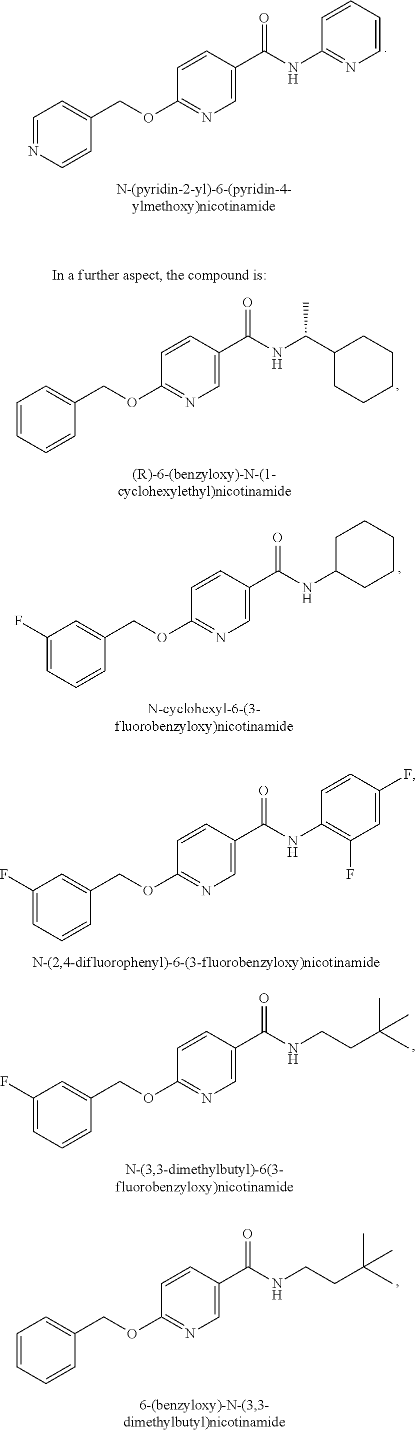

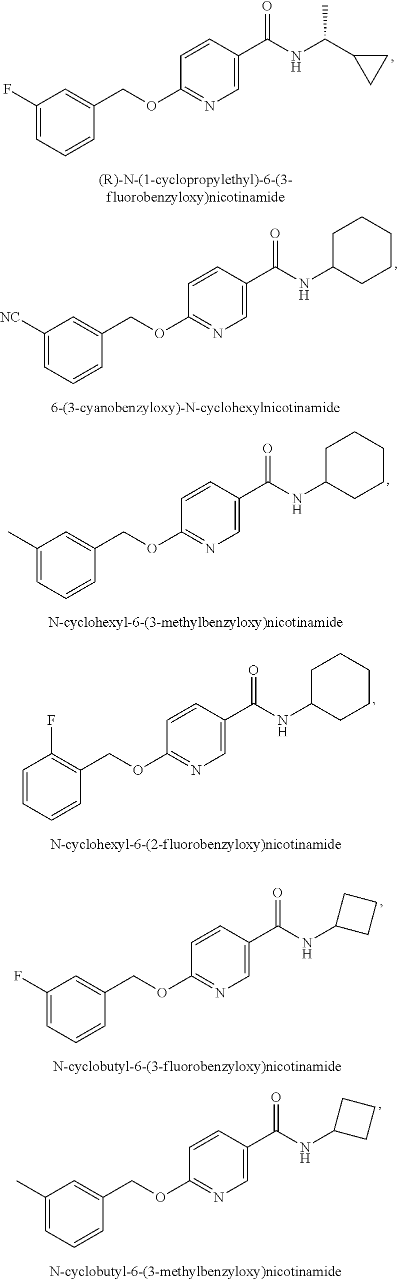

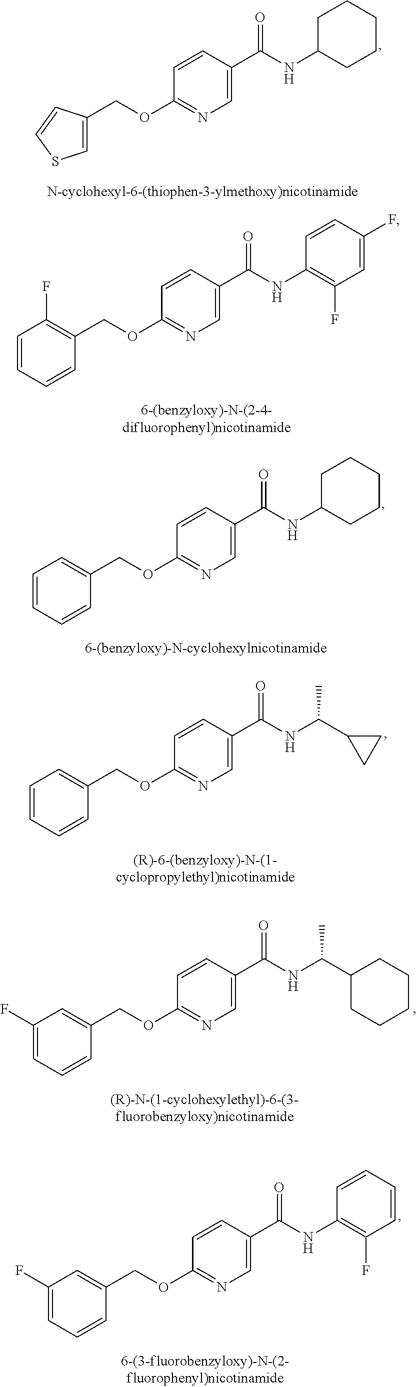

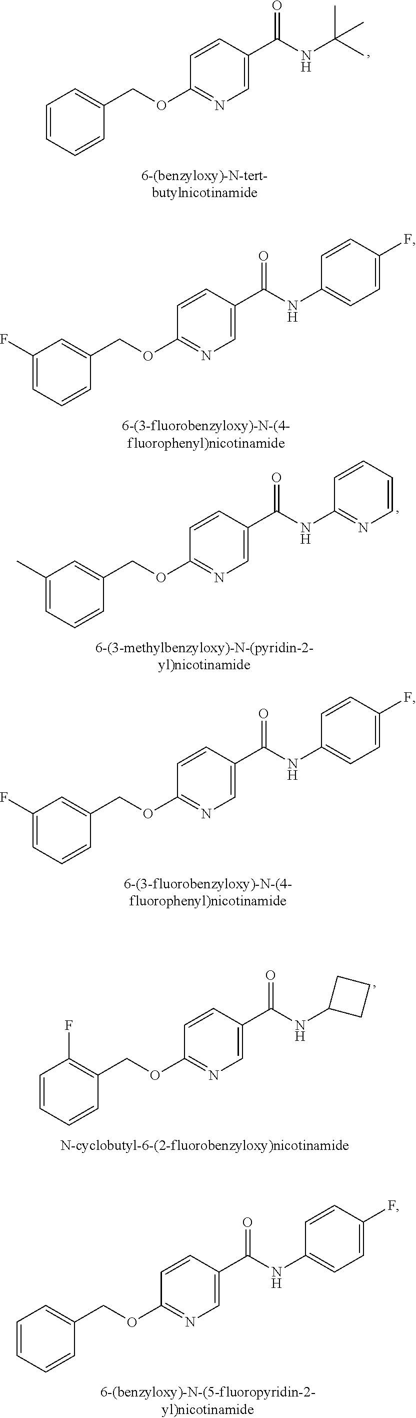

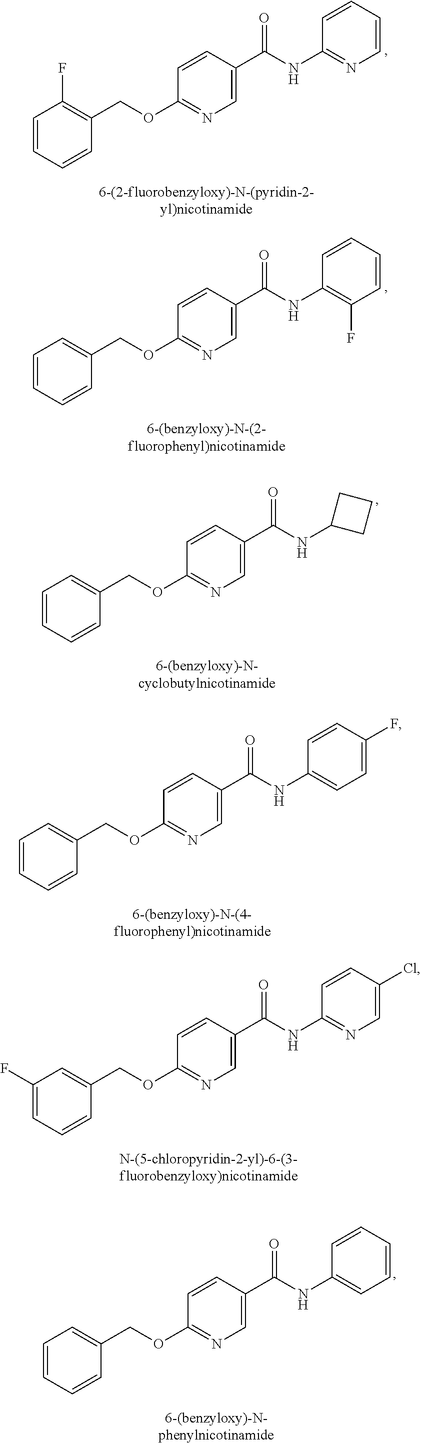

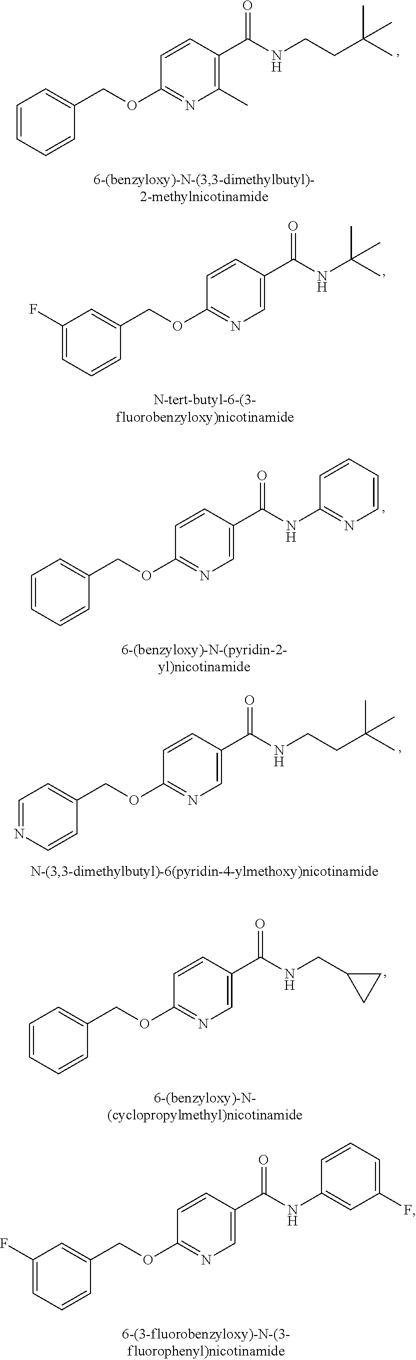

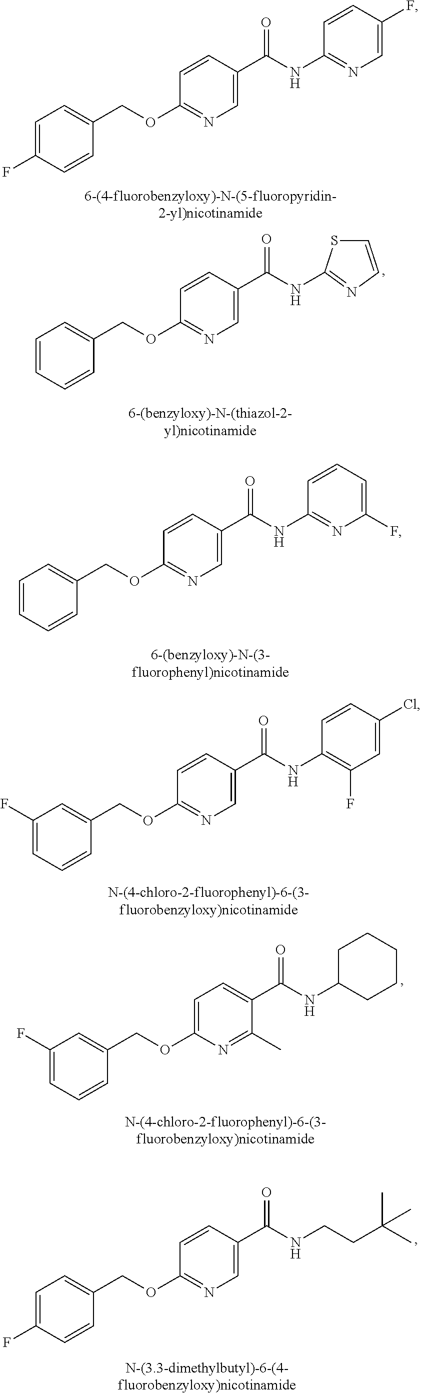

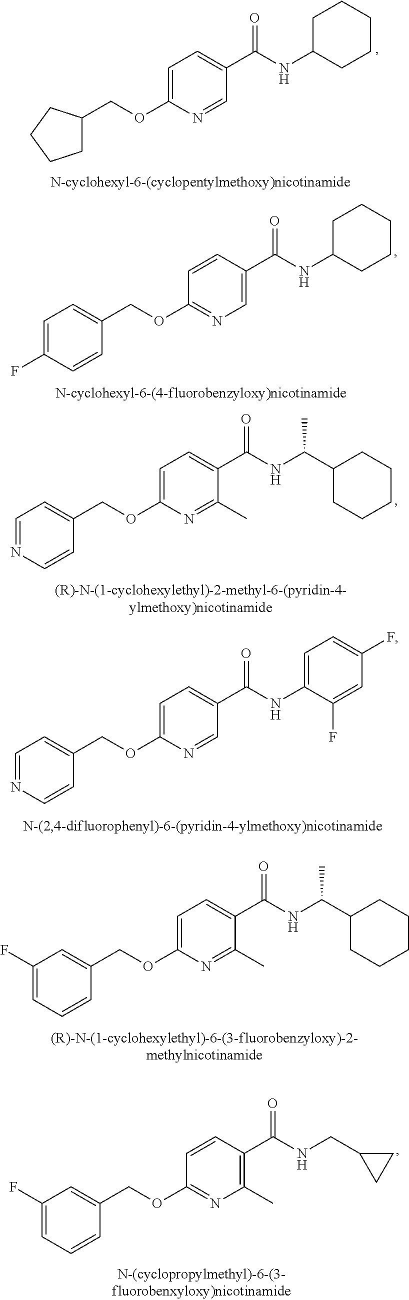

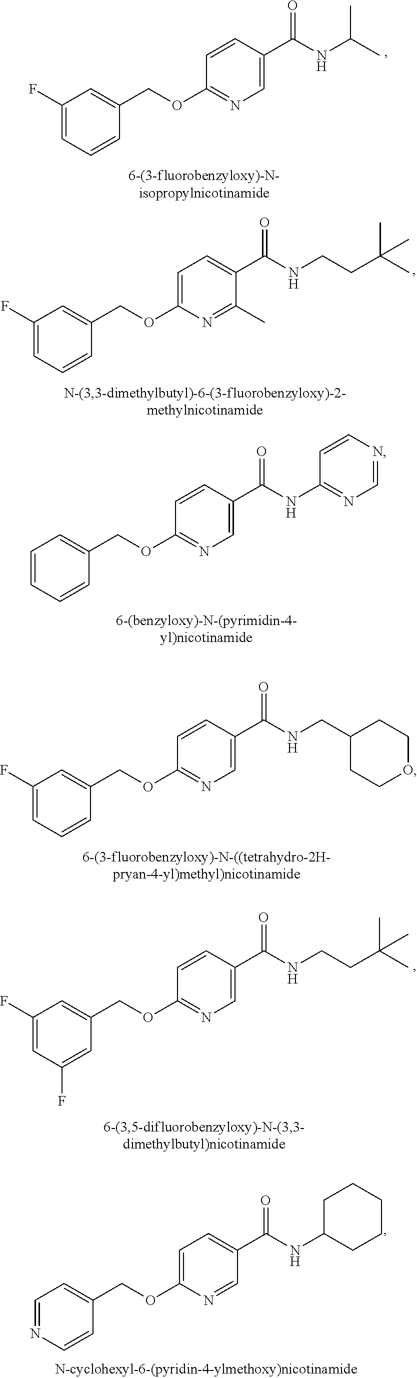

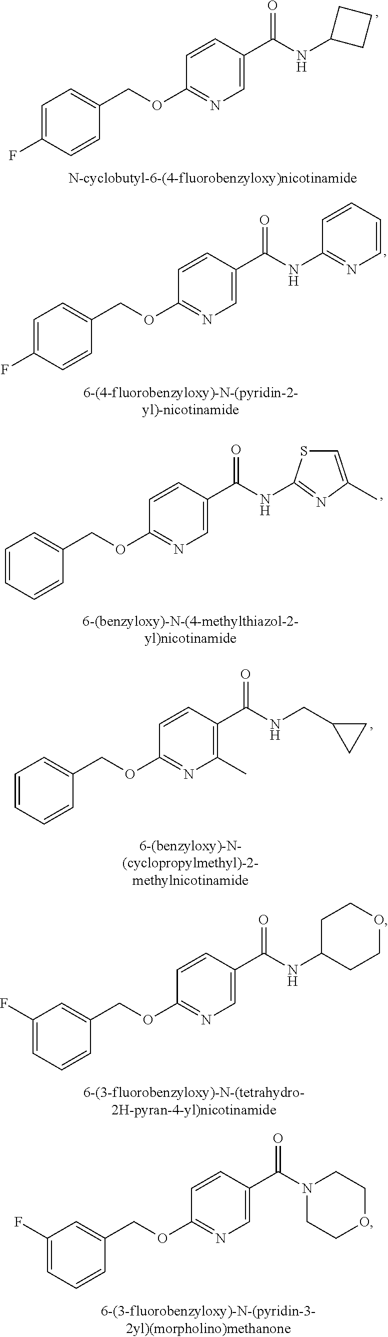

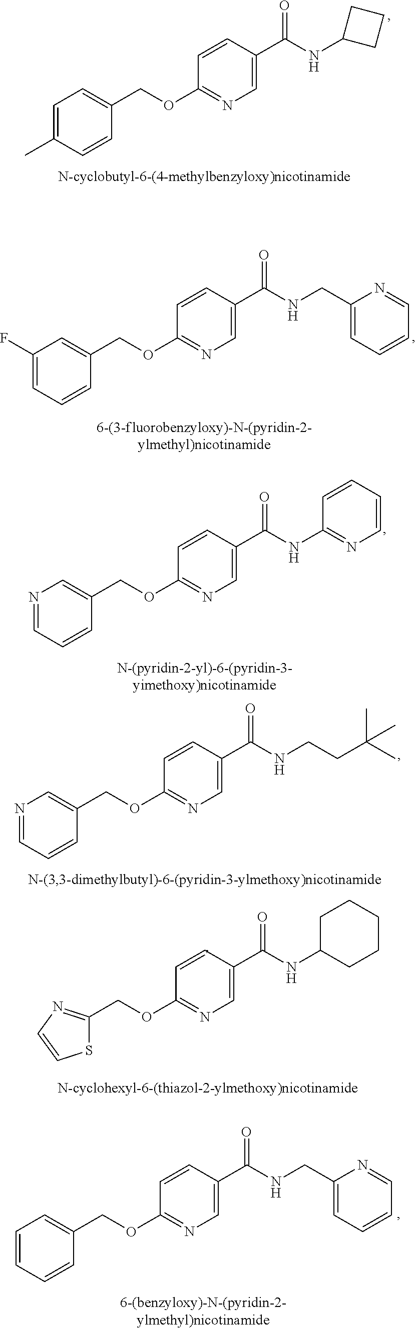

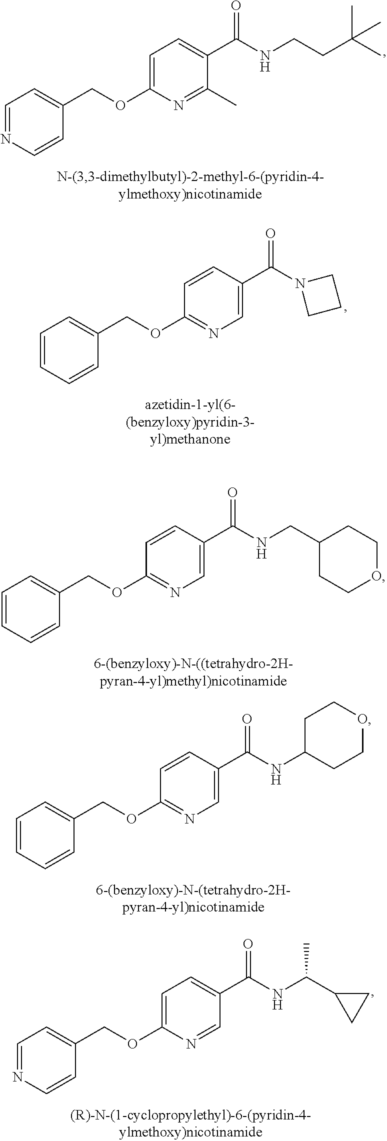

In a further aspect, the compound is:

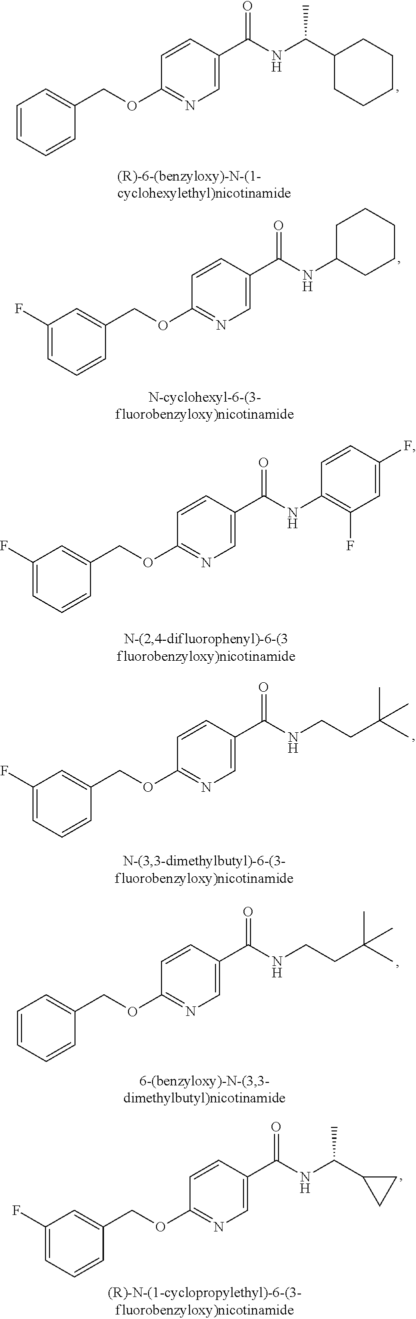

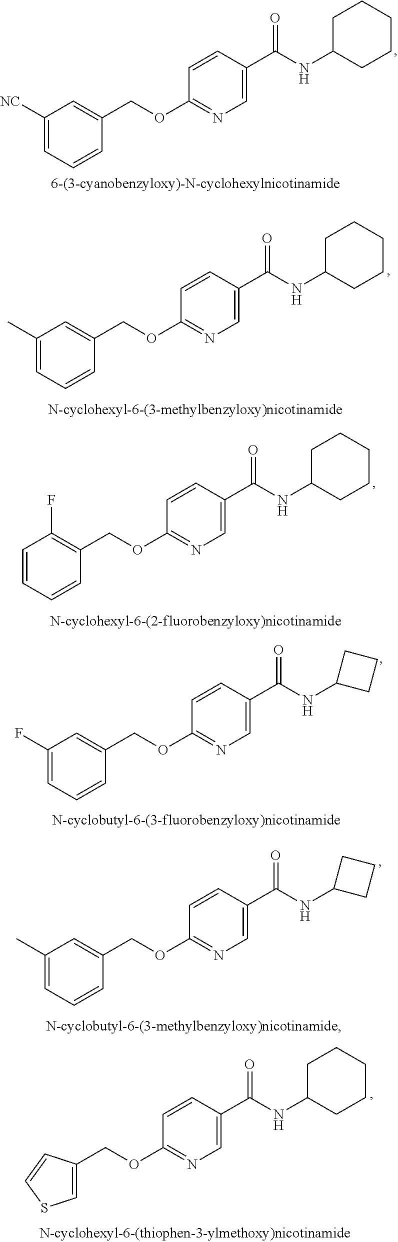

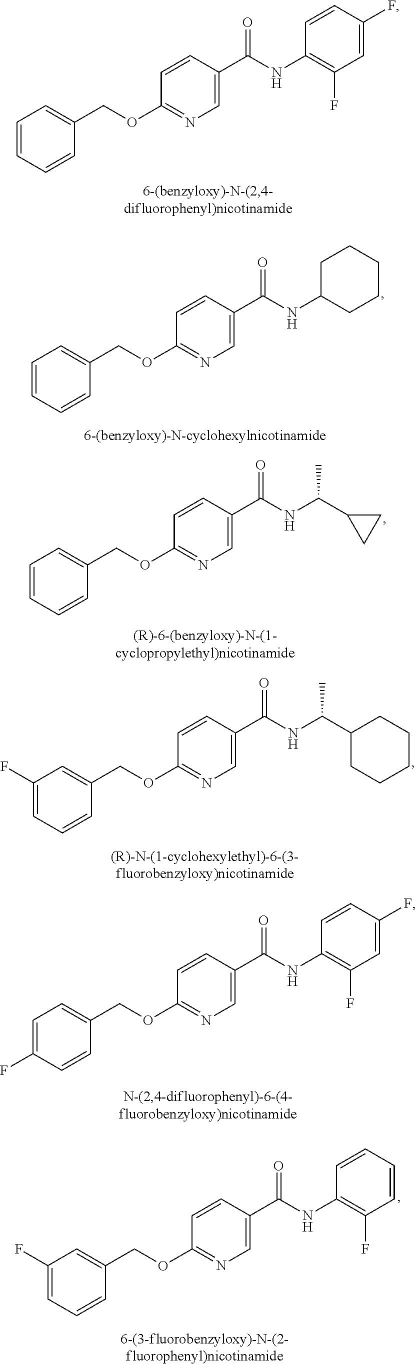

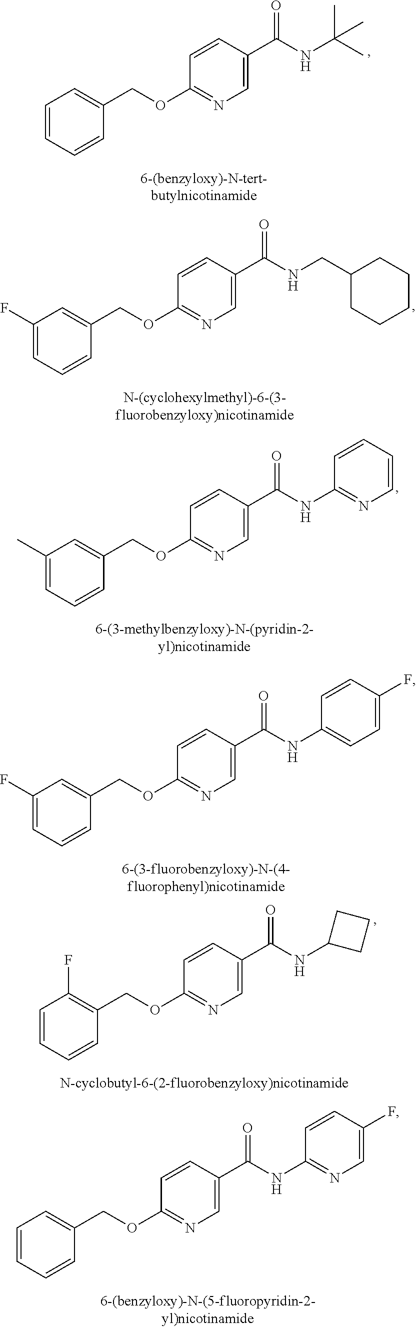

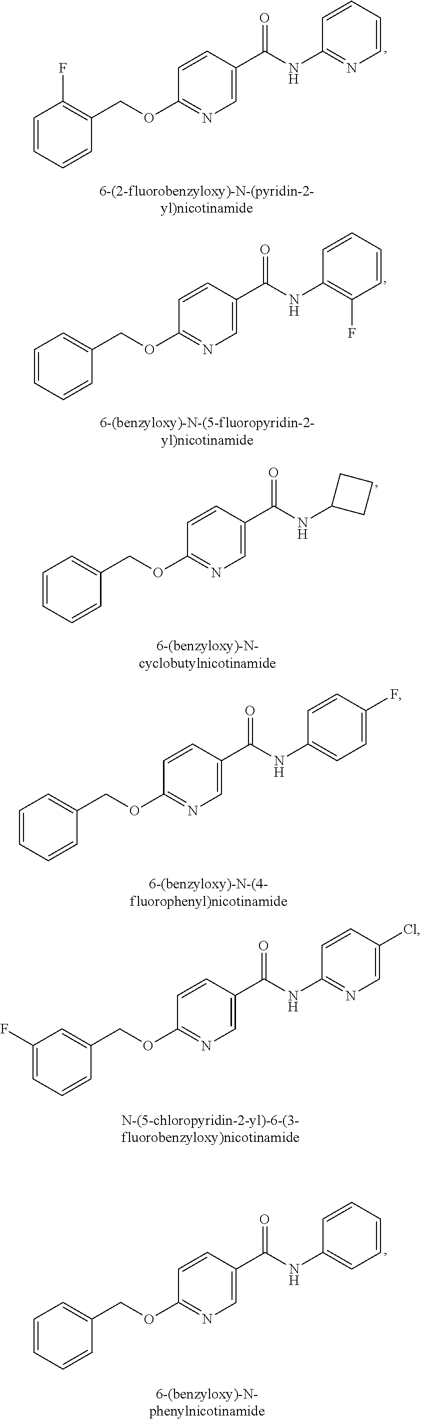

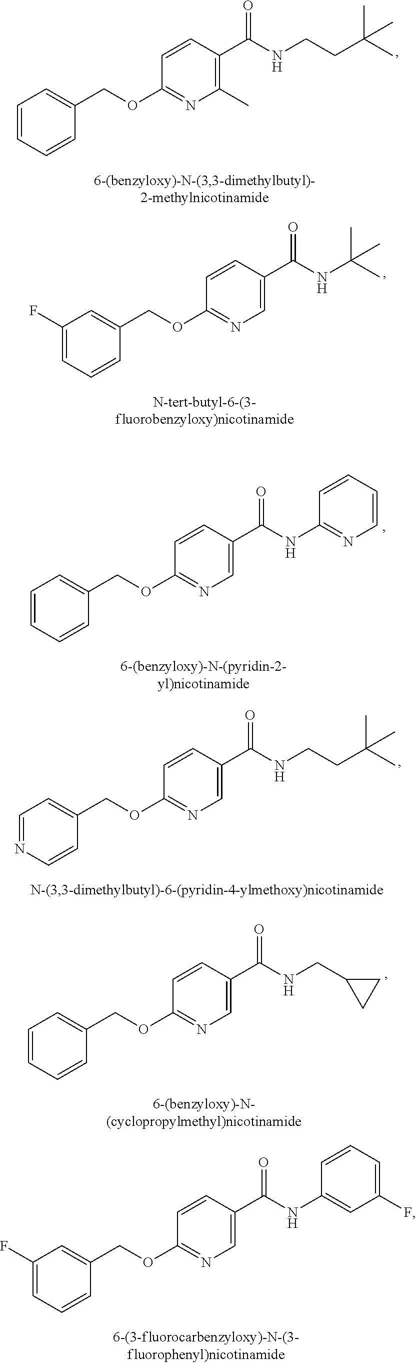

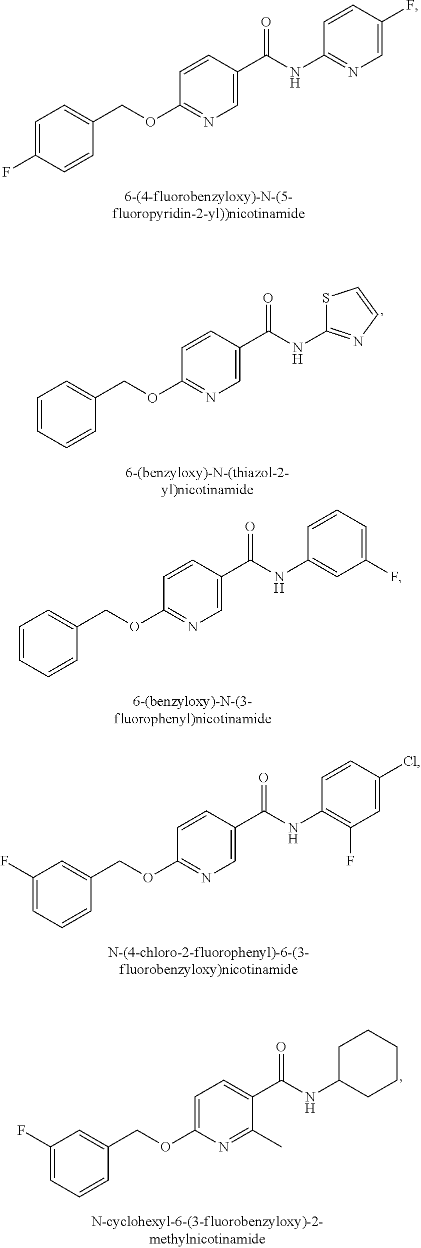

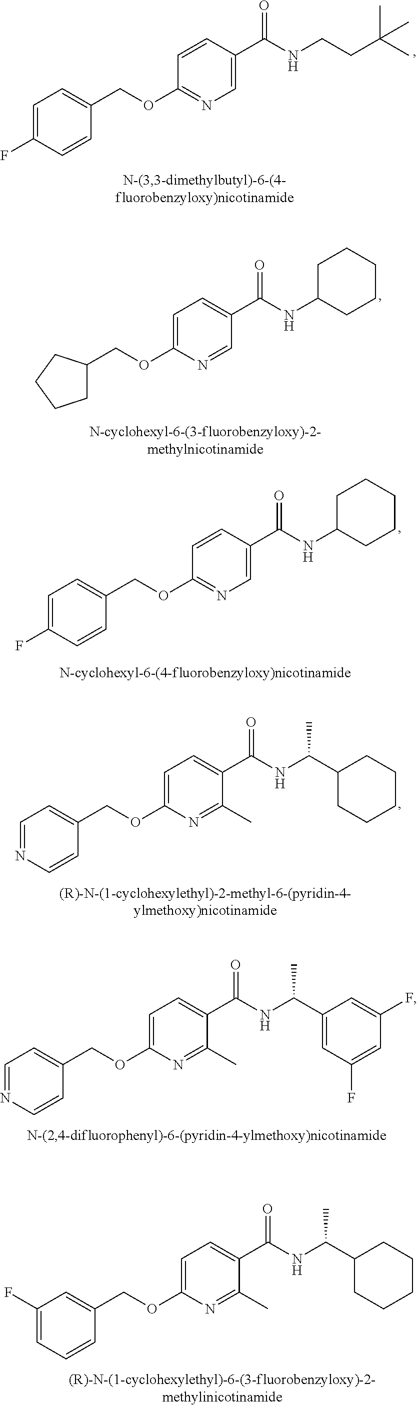

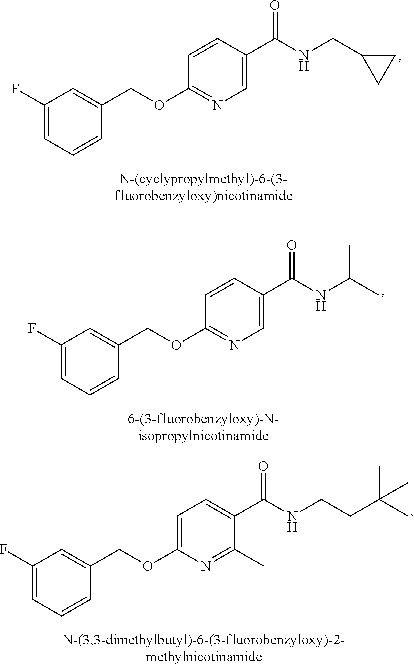

In a further aspect, the compound is:

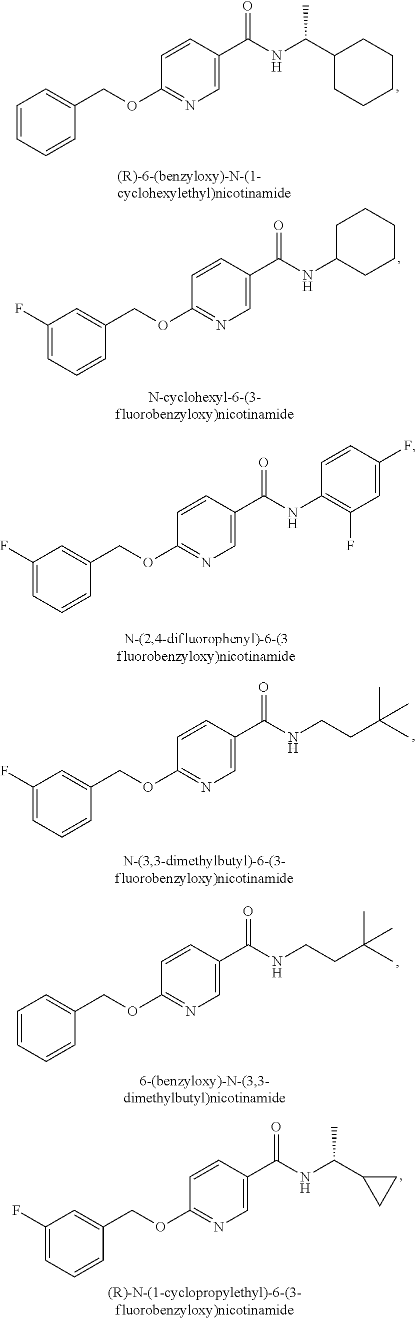

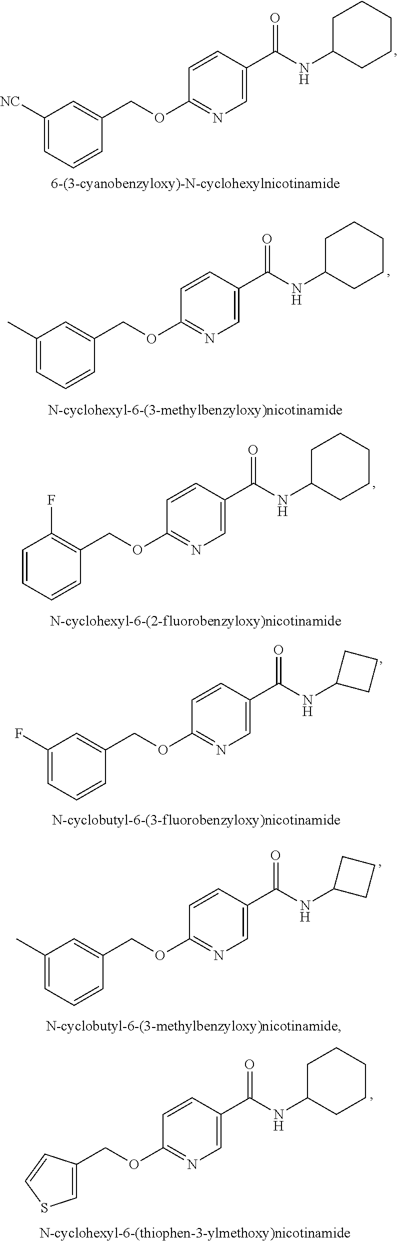

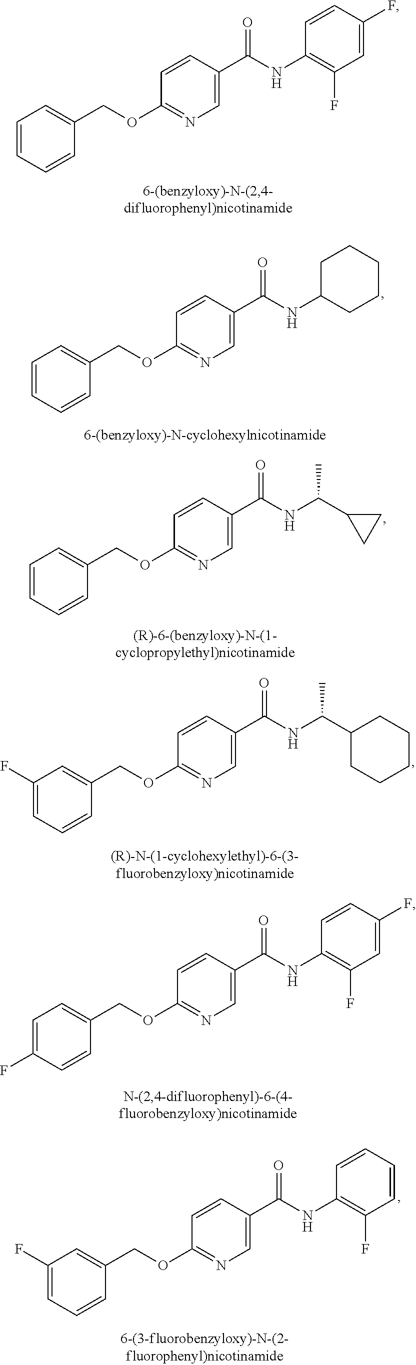

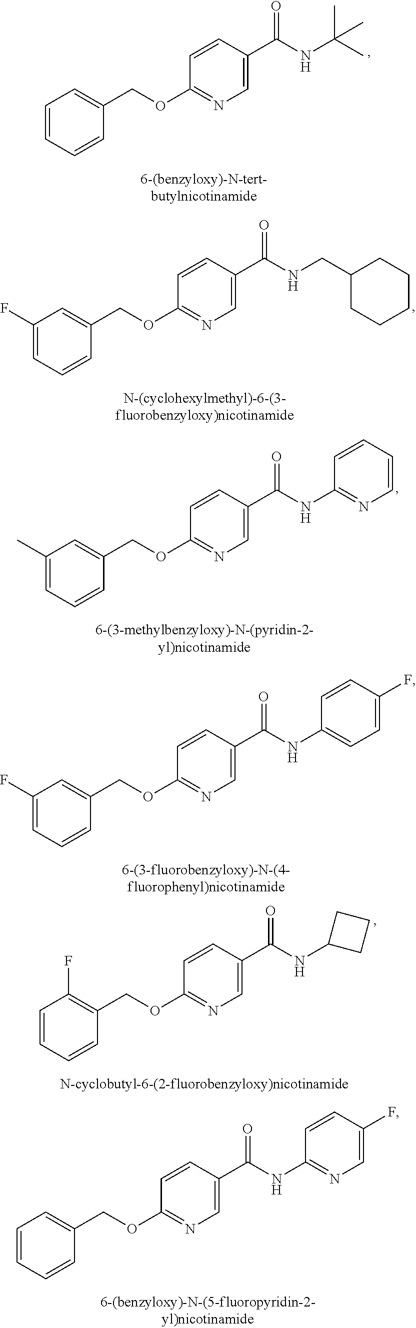

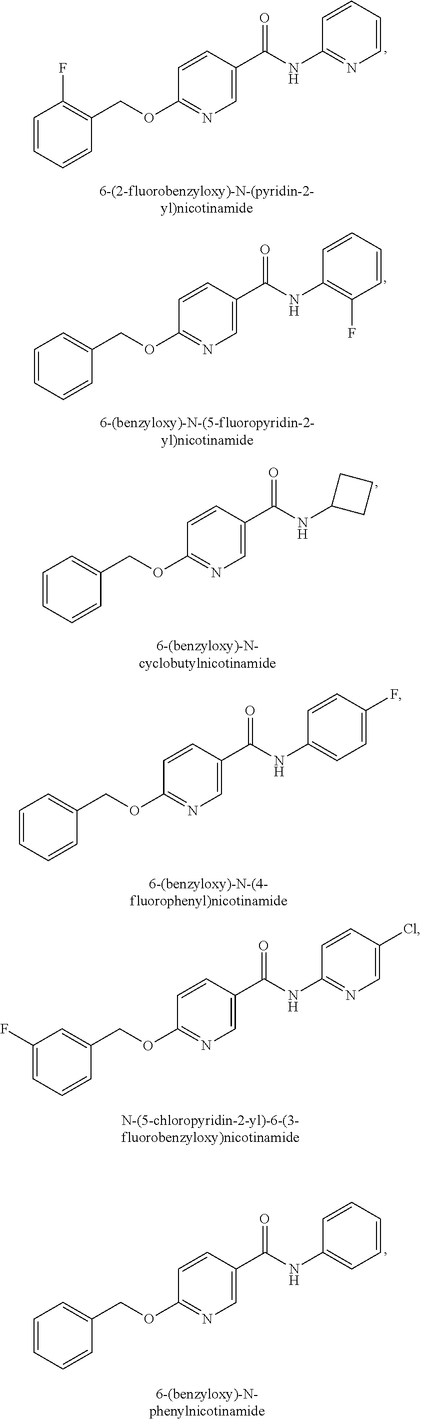

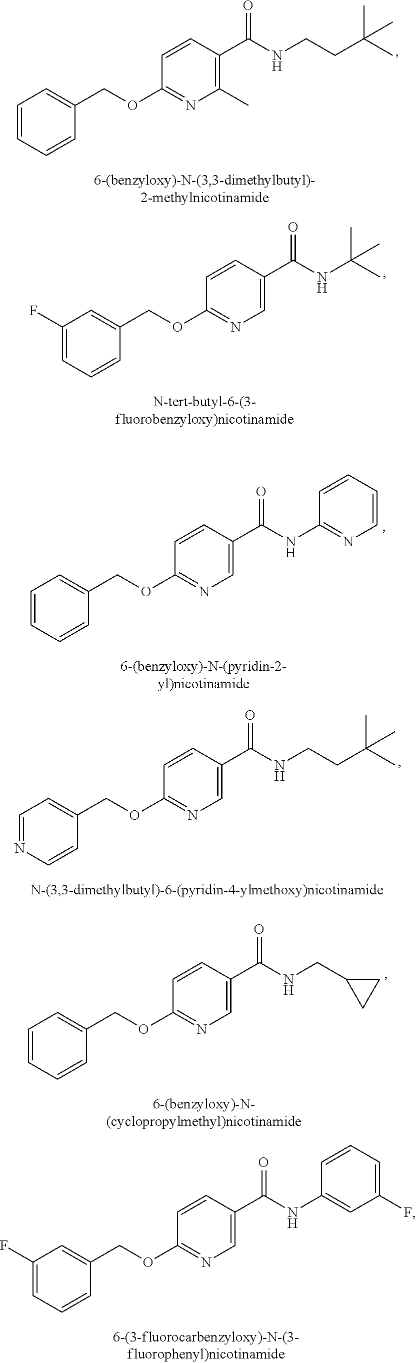

In a further aspect, the compound is:

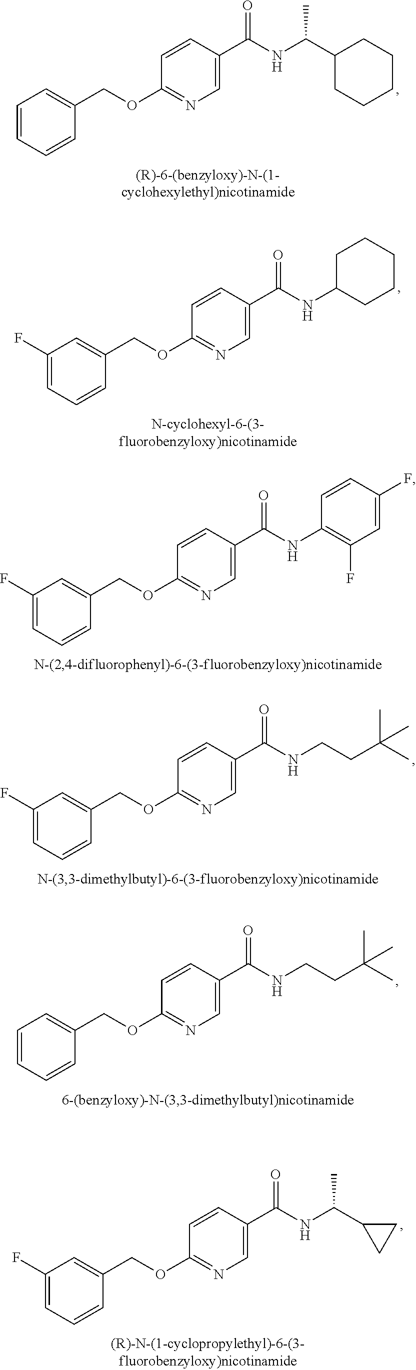

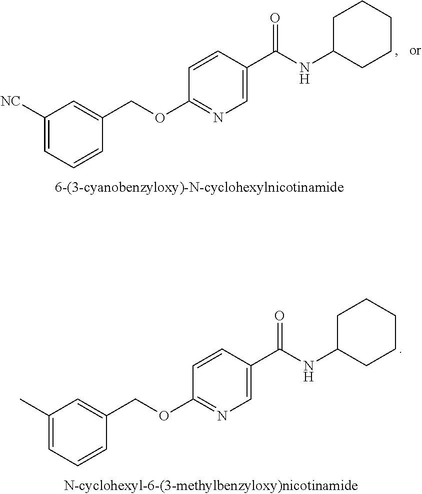

In a further aspect, the compound is:

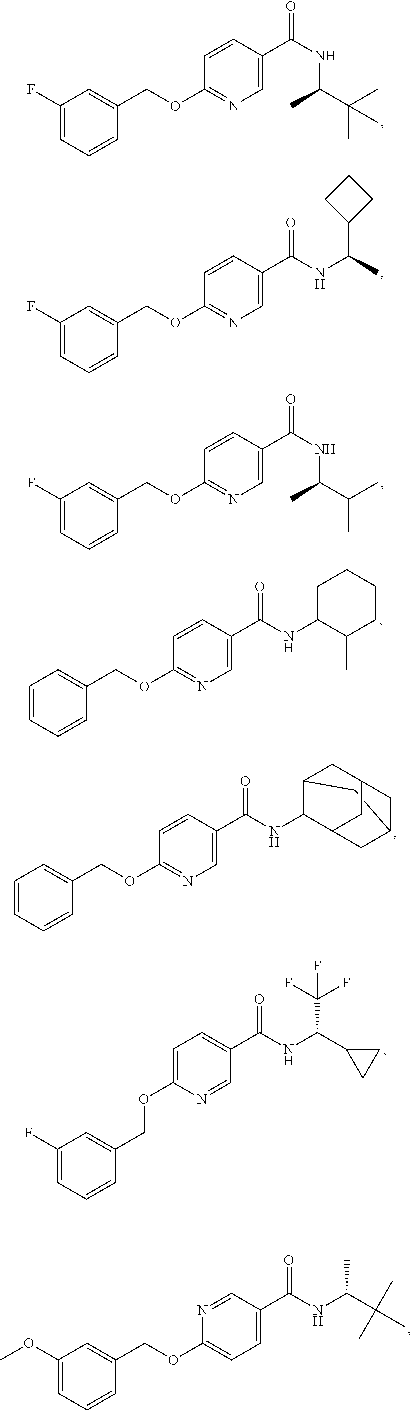

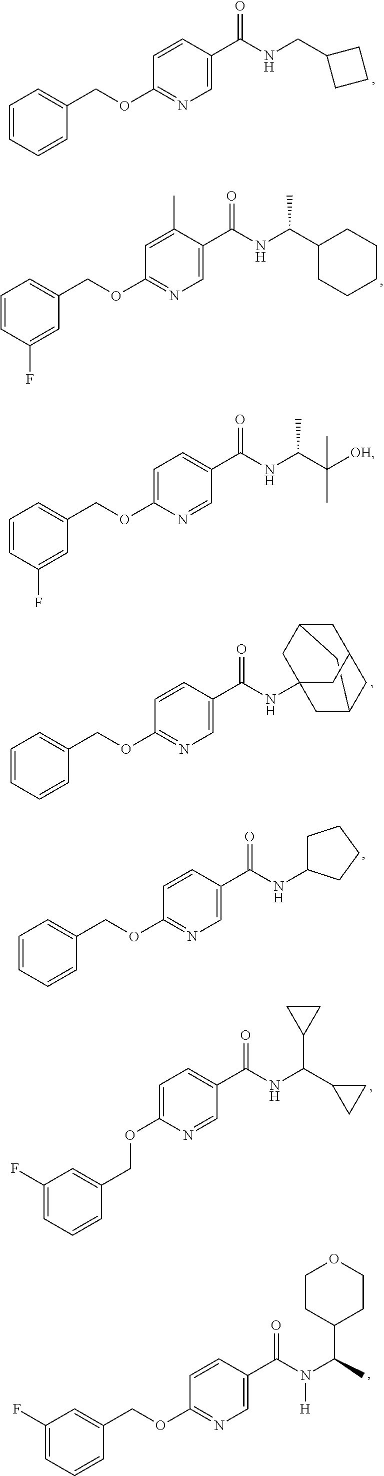

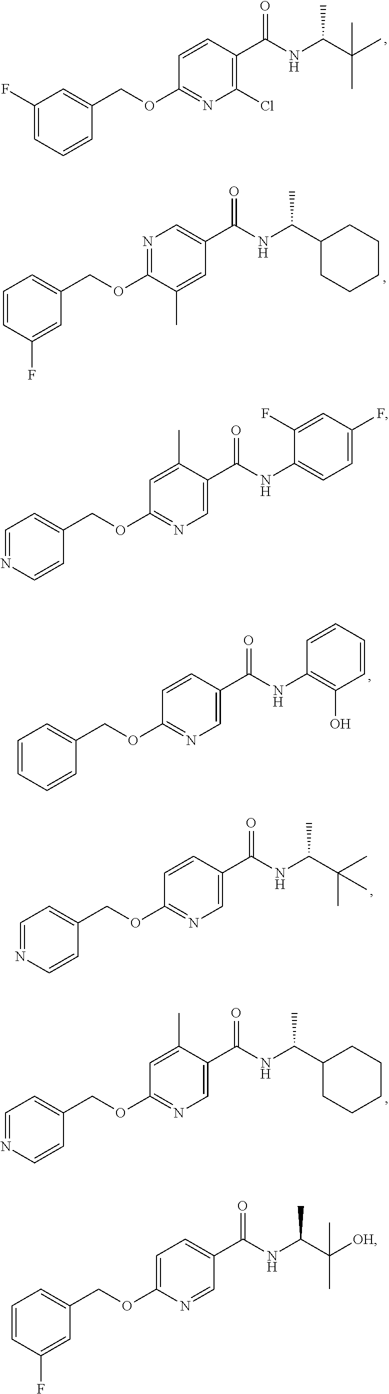

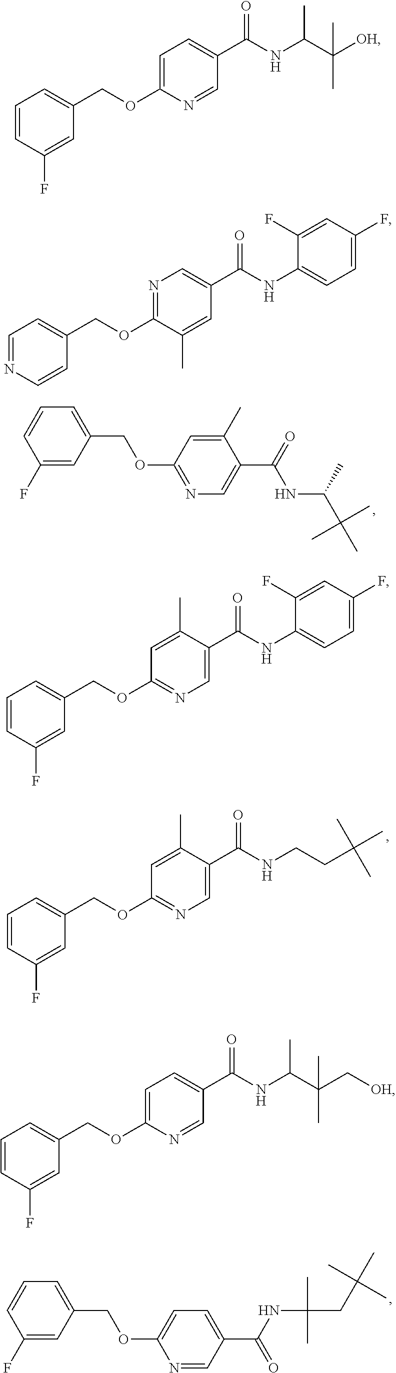

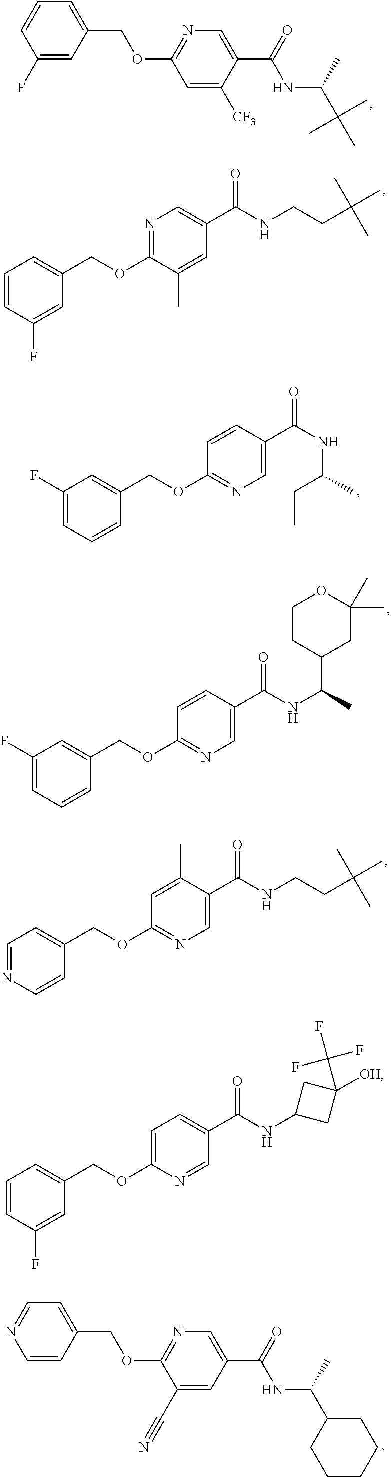

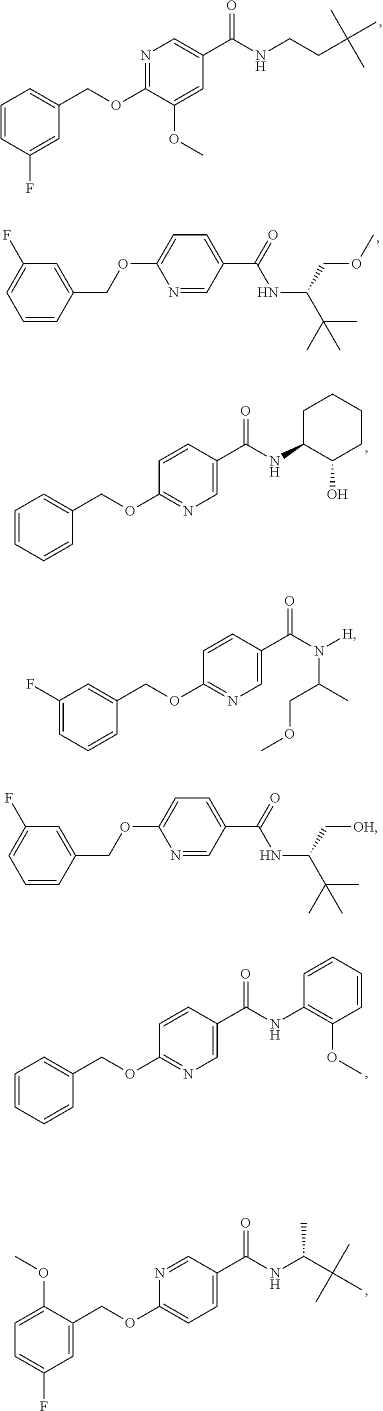

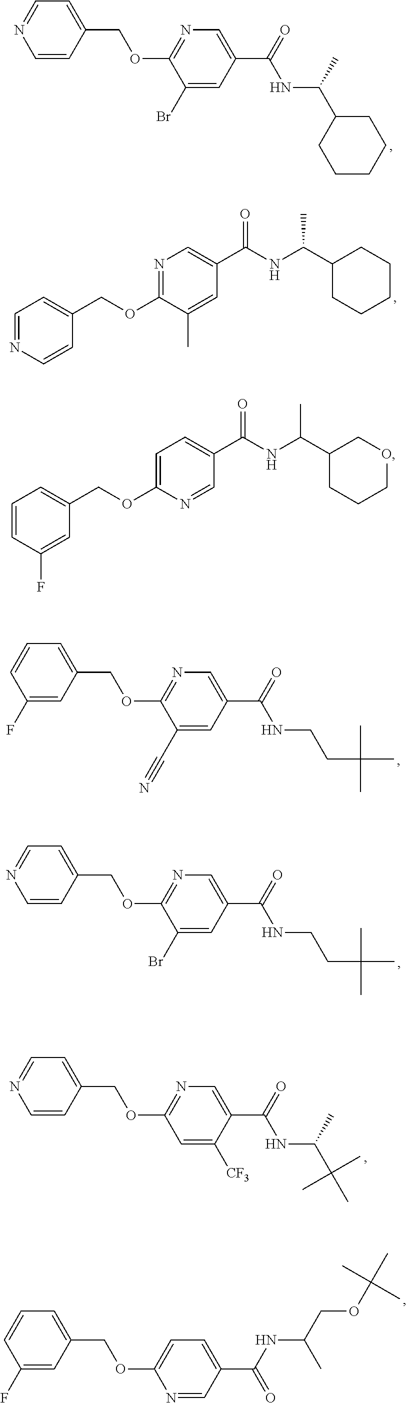

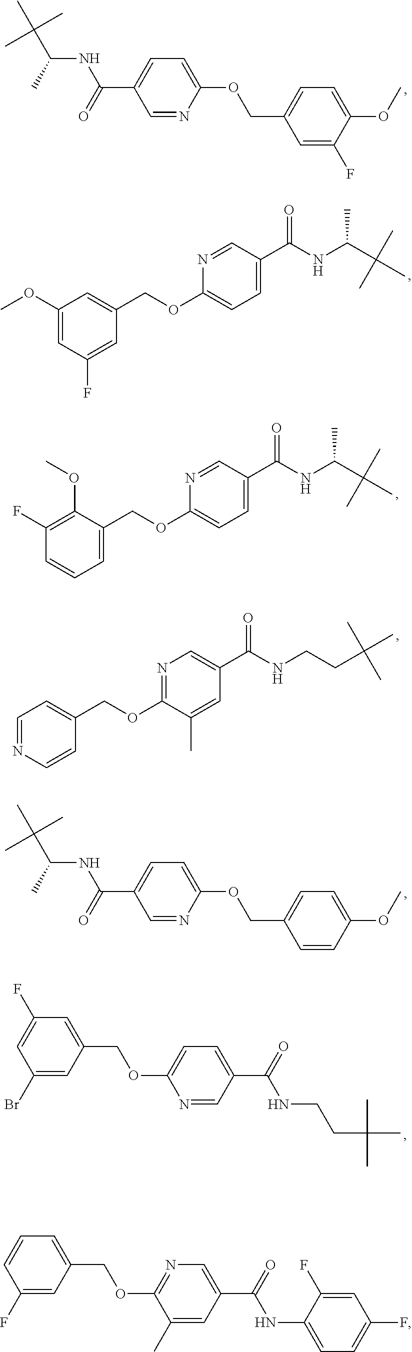

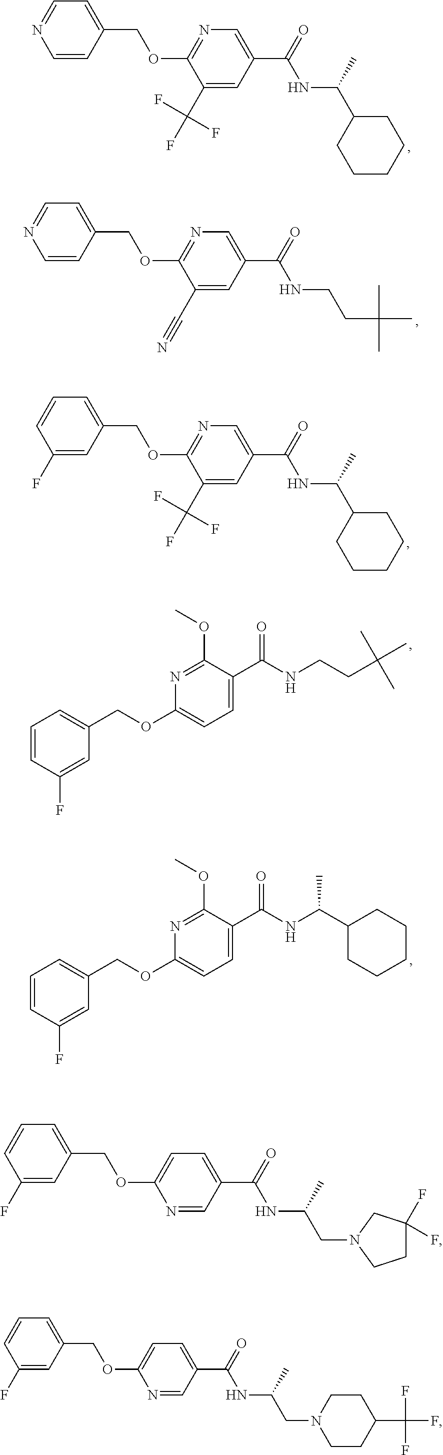

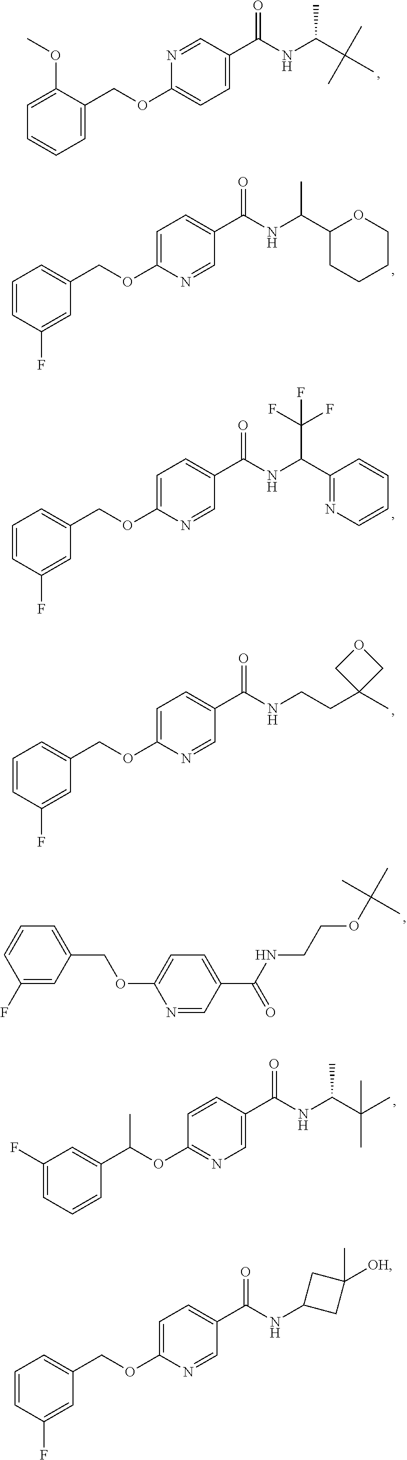

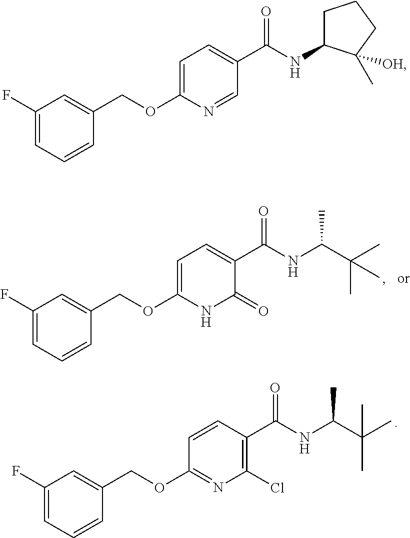

In a further aspect, the compound is:

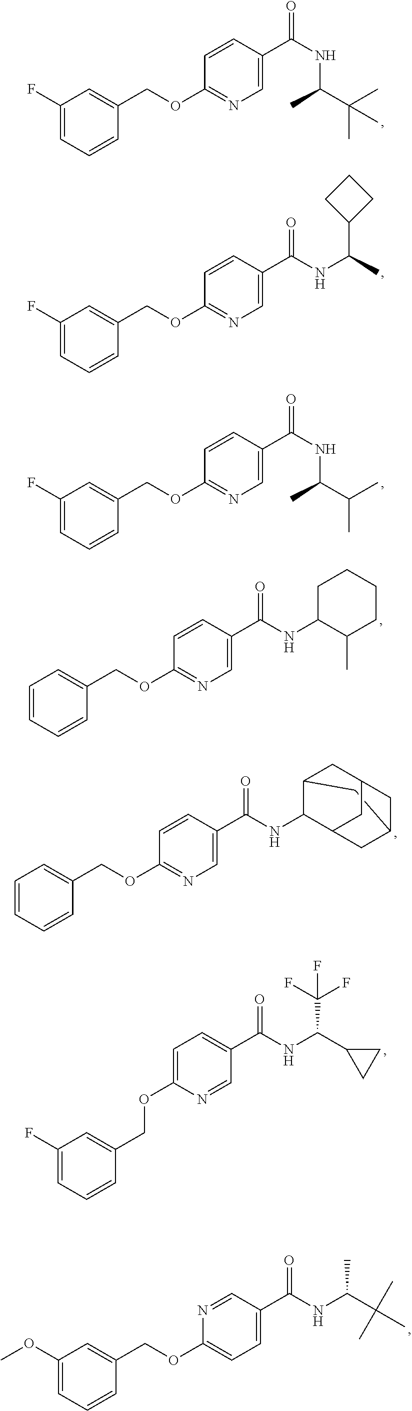

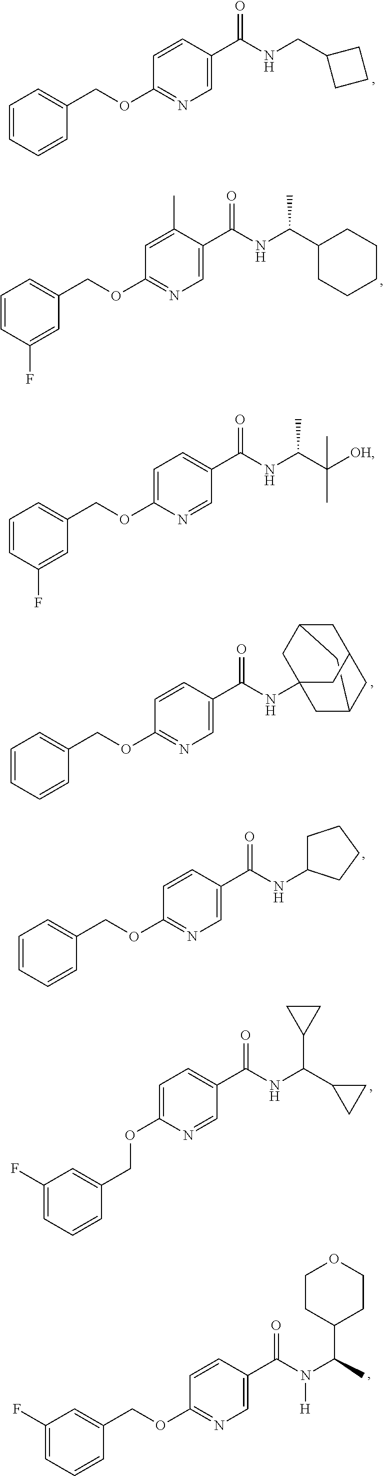

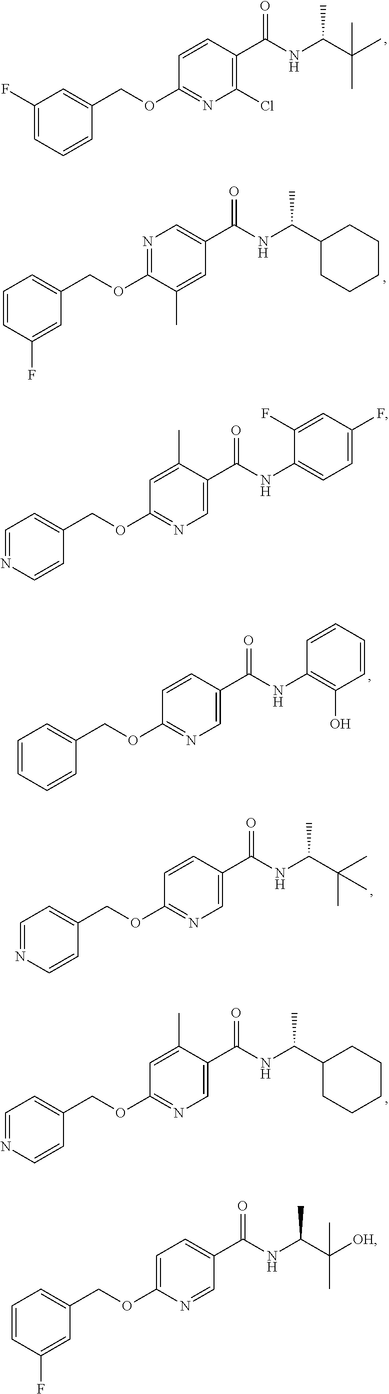

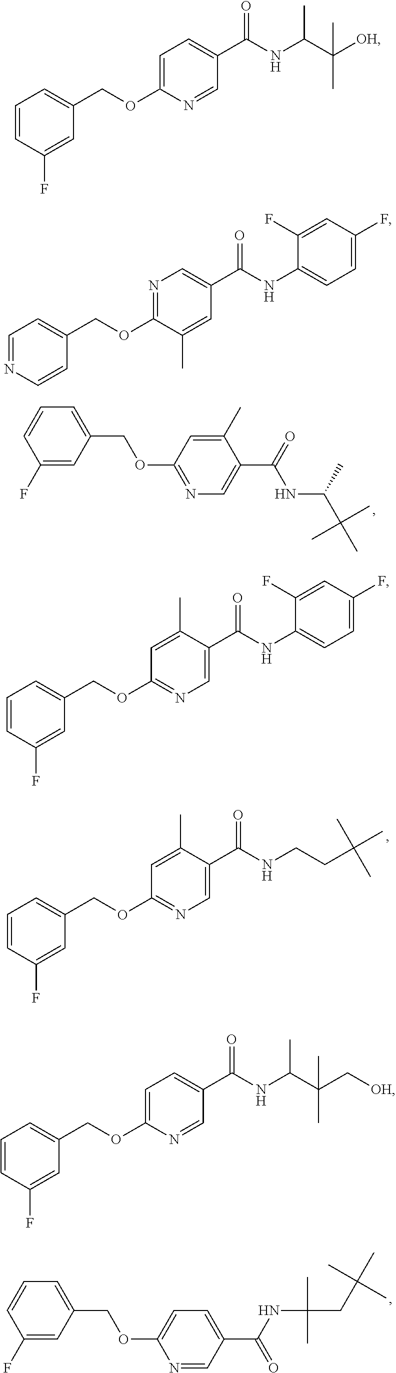

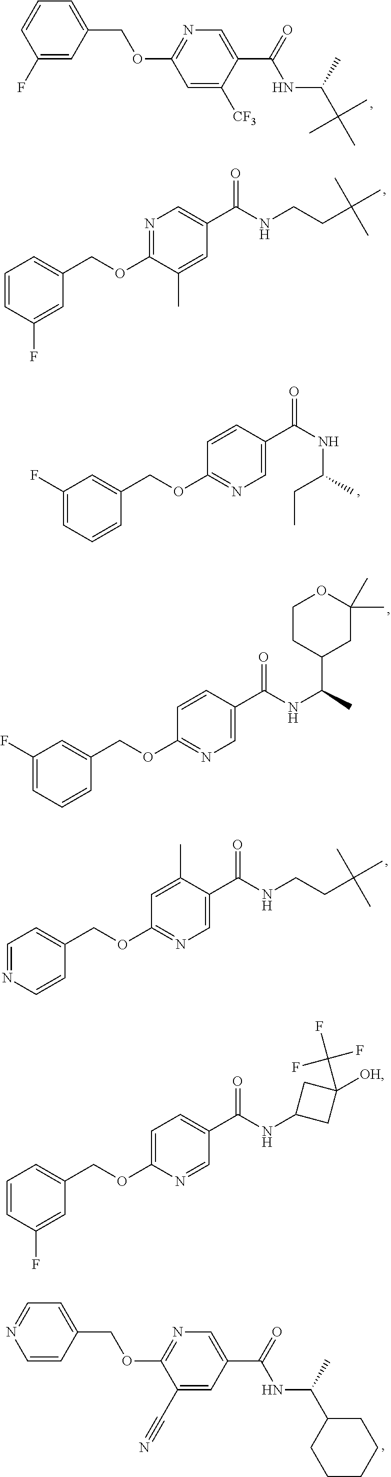

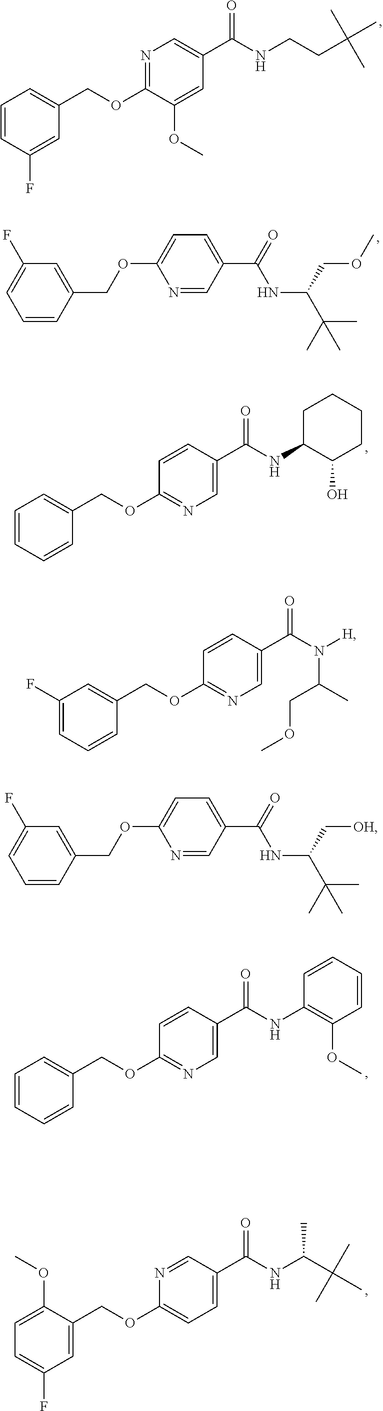

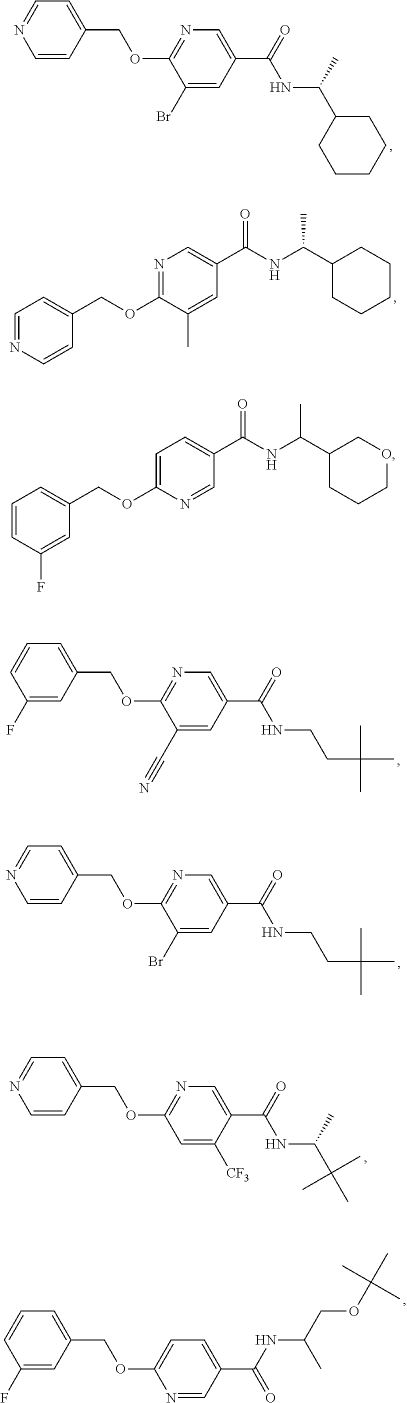

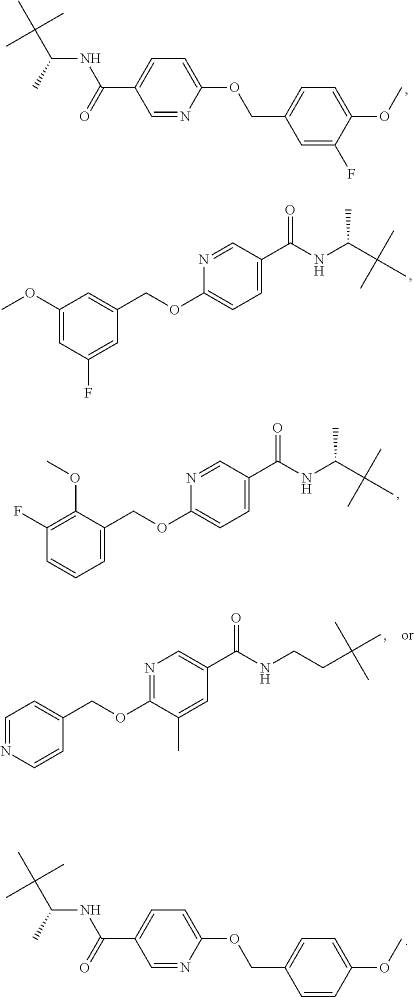

In a further aspect, the compound is:

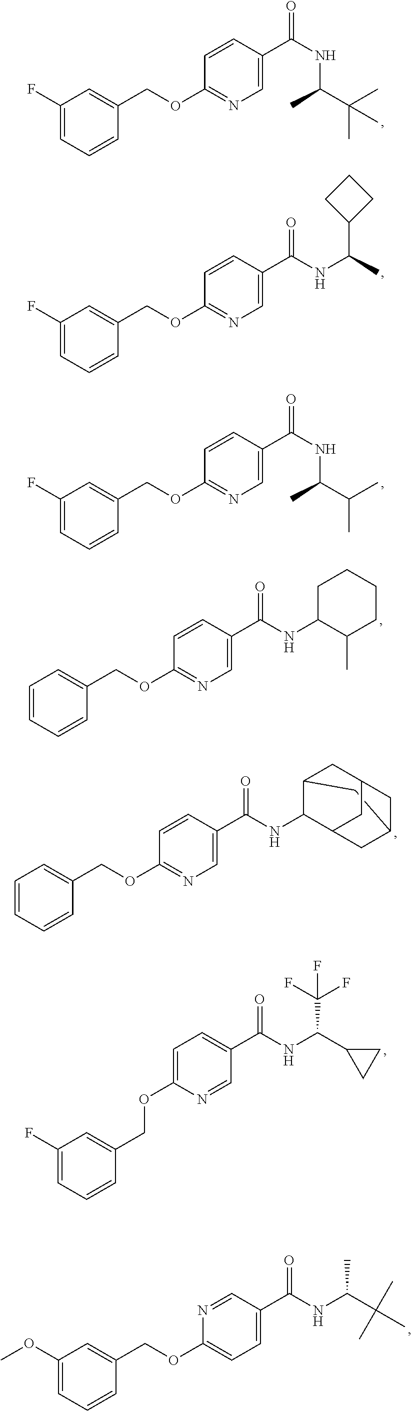

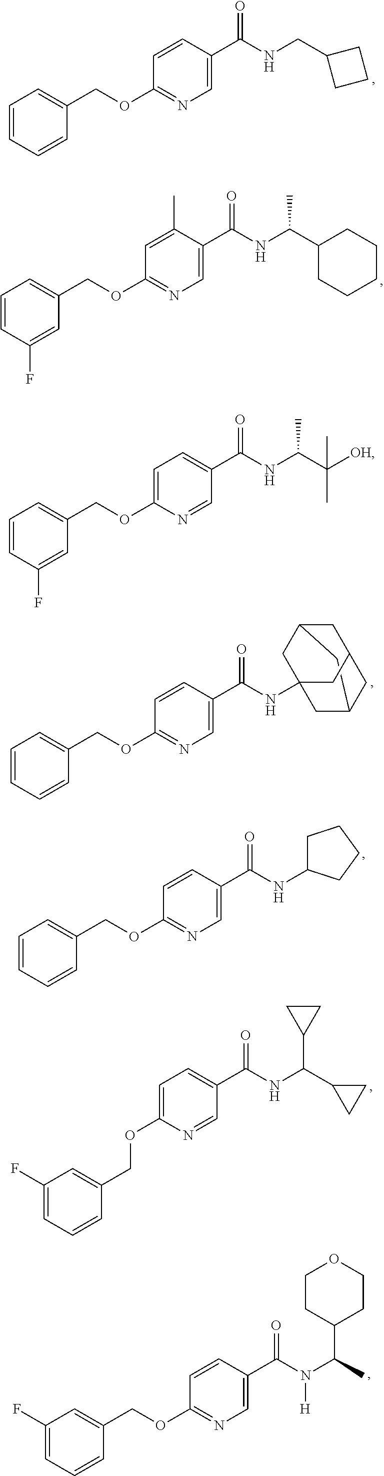

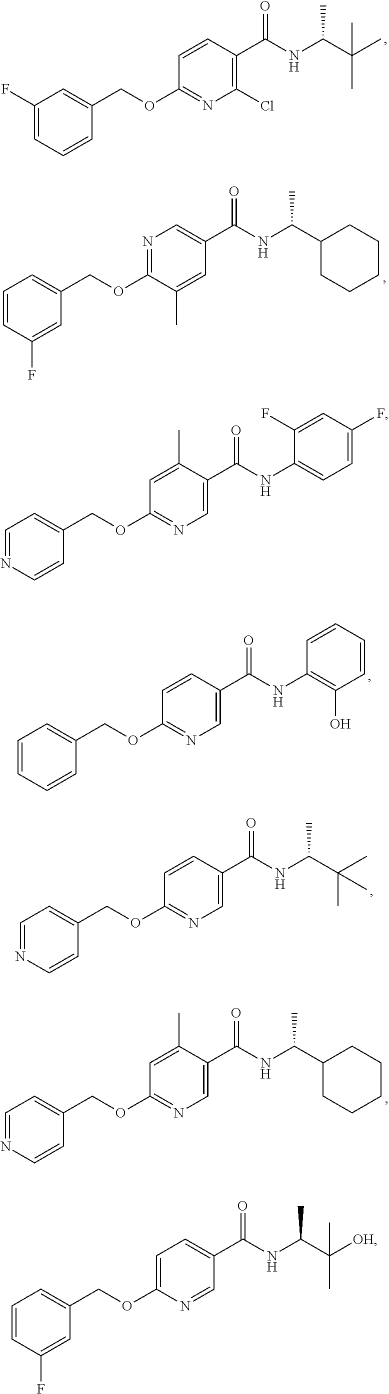

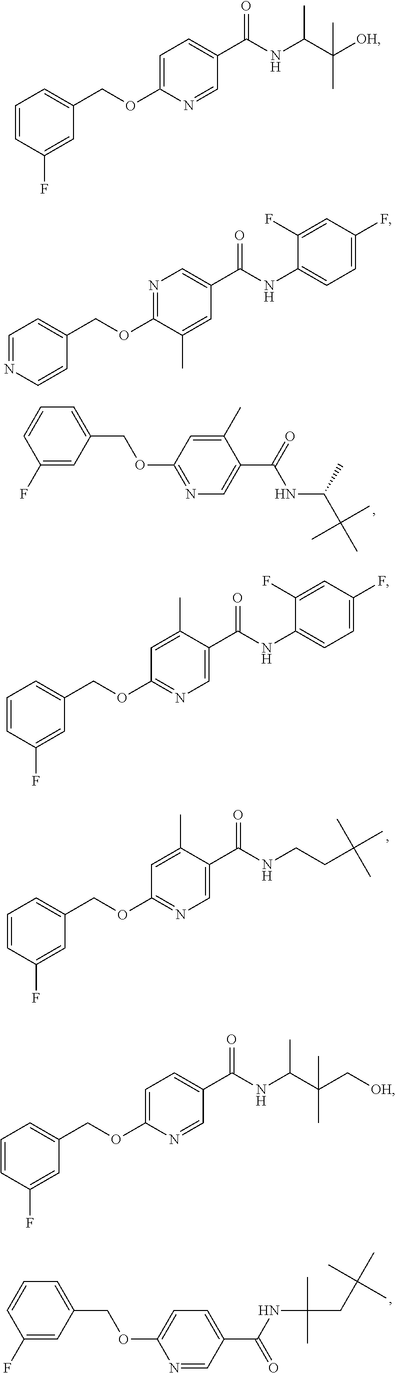

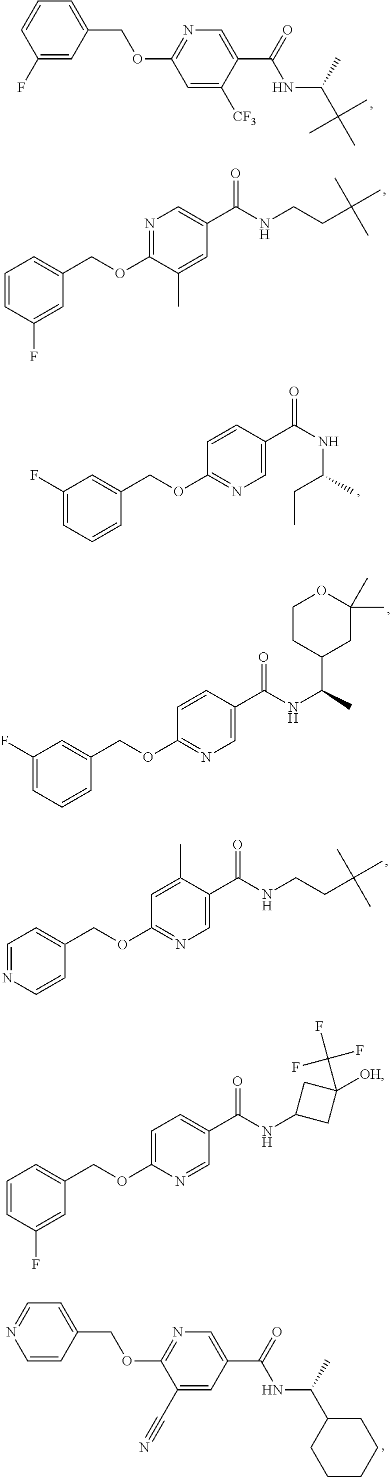

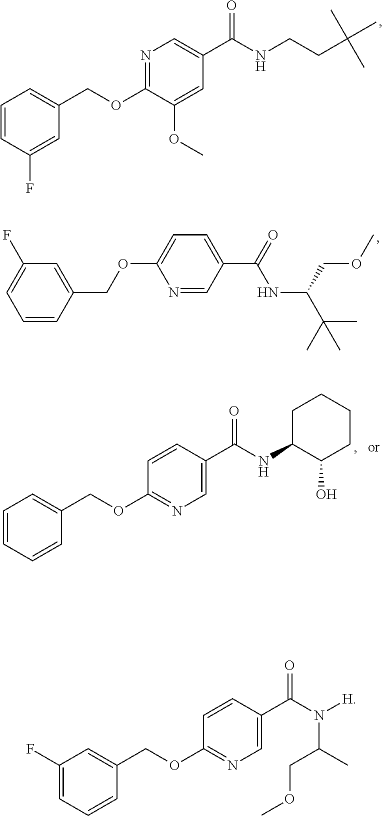

In a further aspect, the compound is:

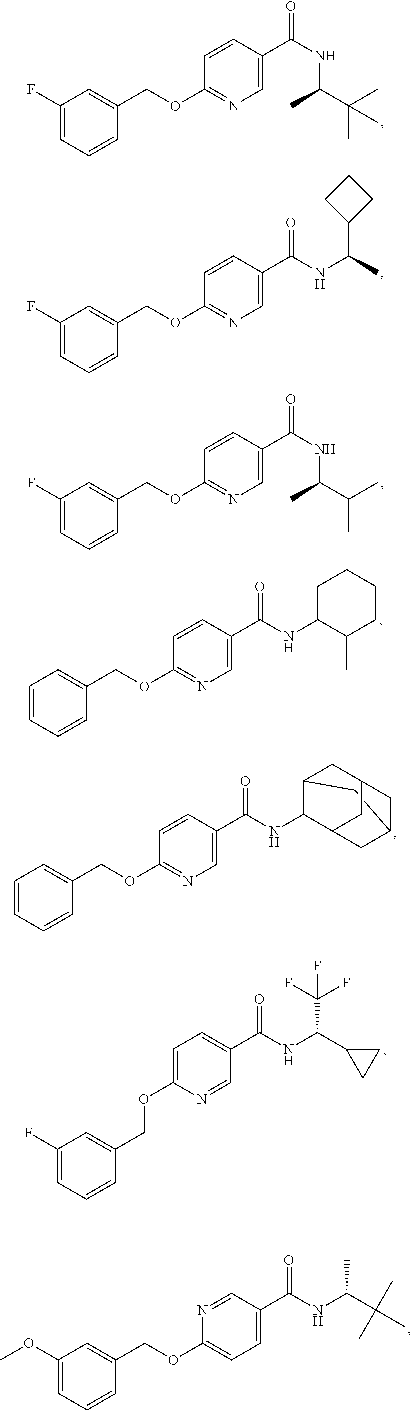

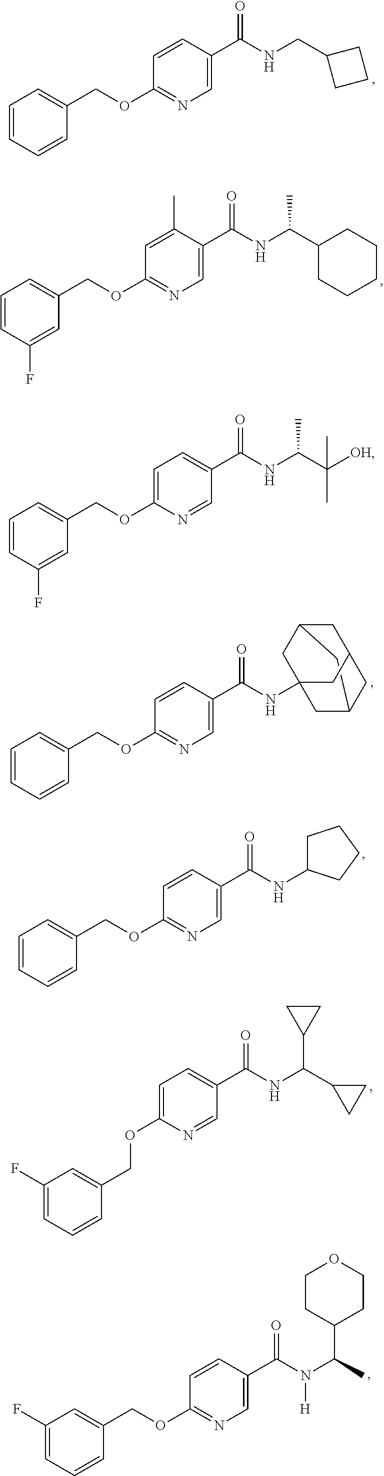

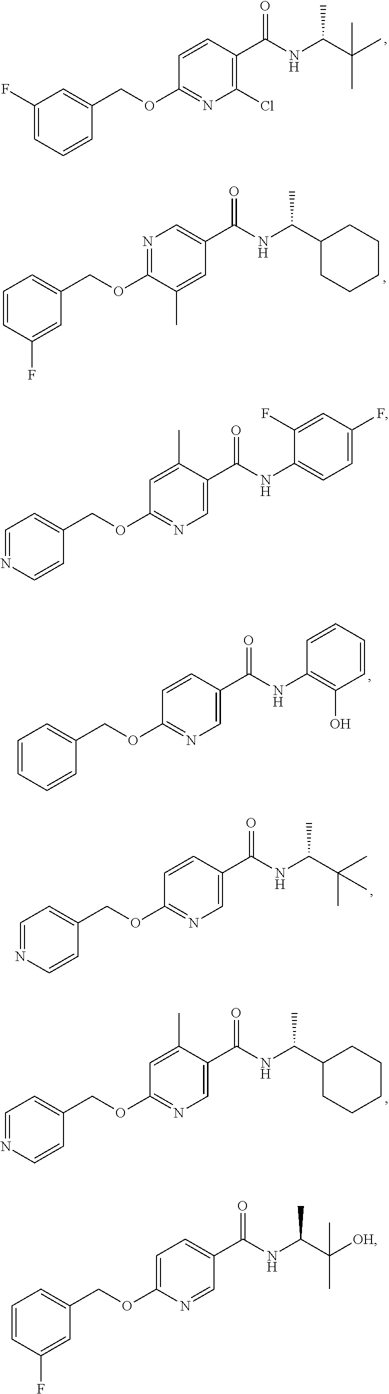

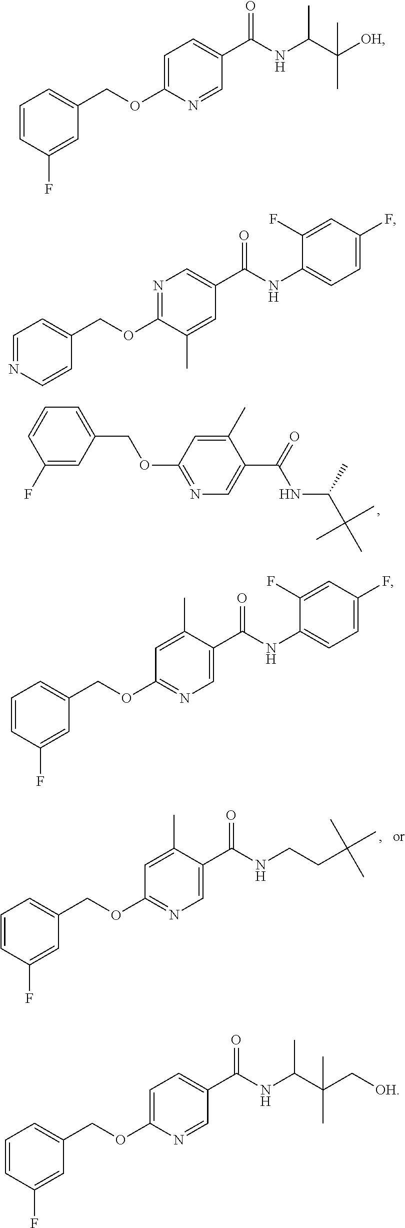

In a further aspect, the compound is:

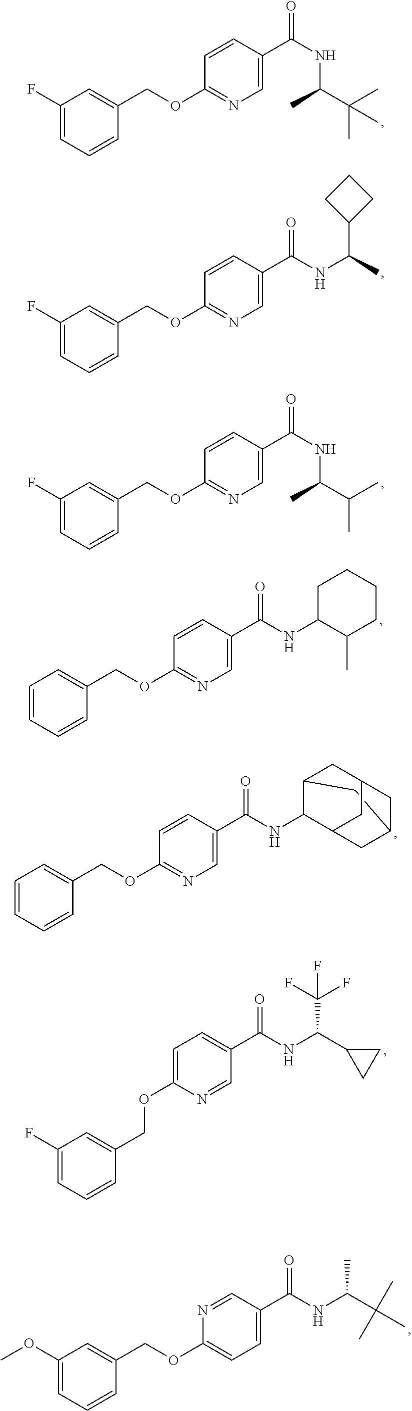

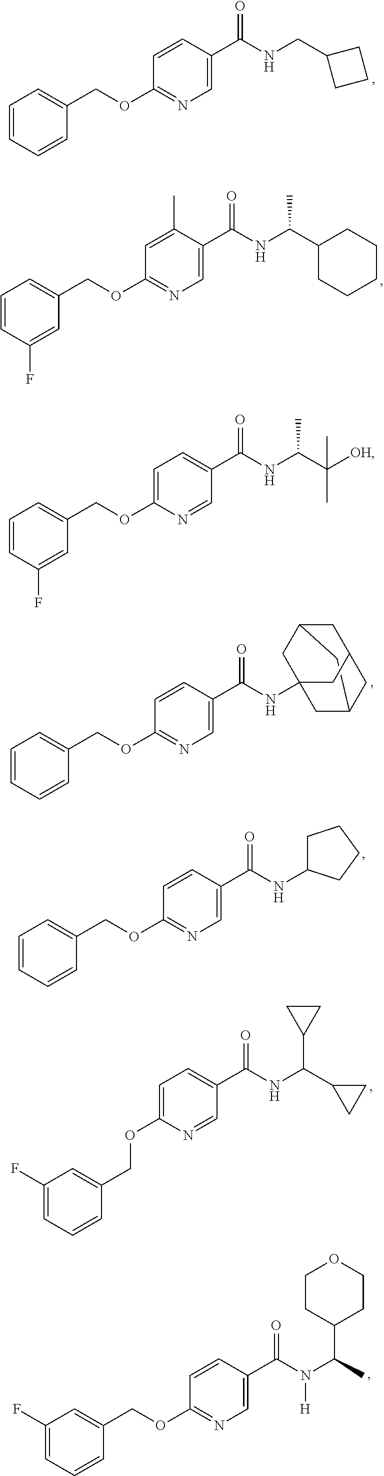

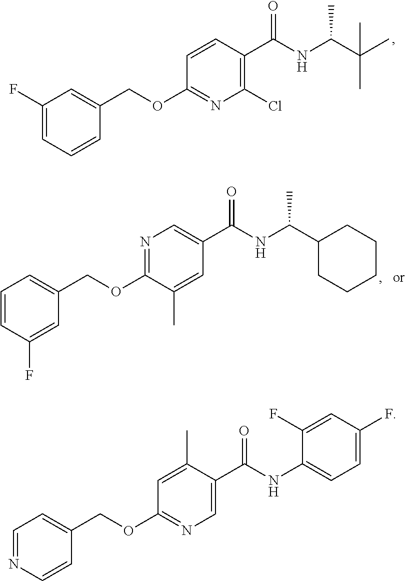

In a further aspect, the compound is:

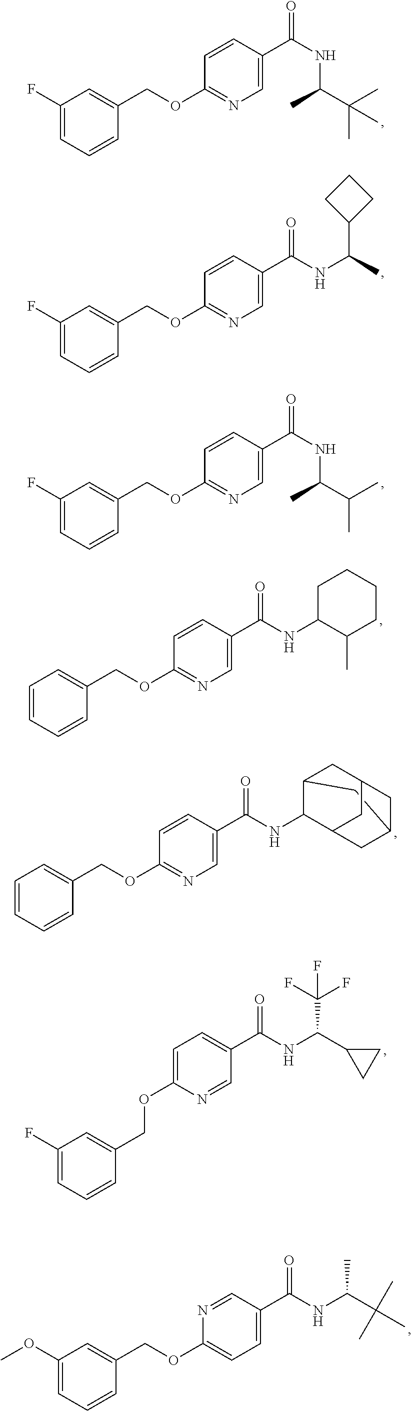

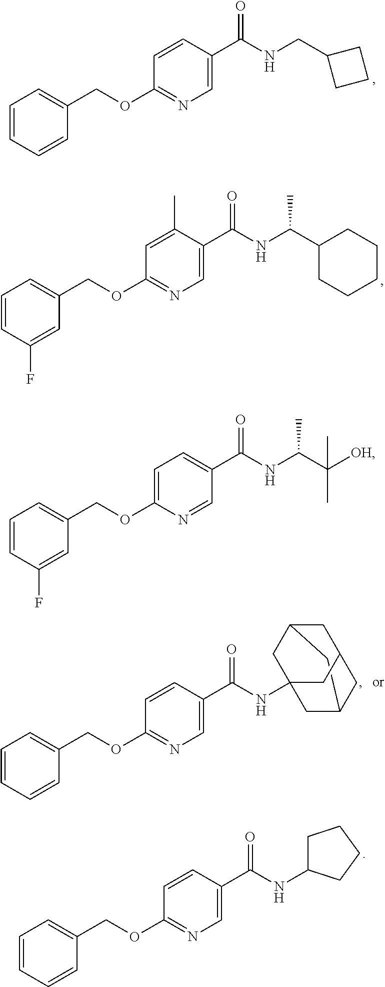

In a further aspect, the compound is:

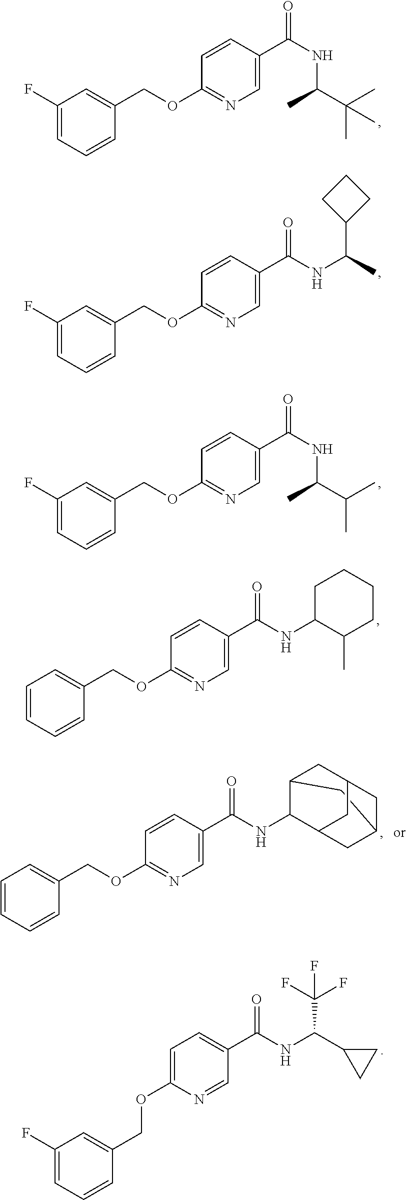

In a further aspect, the compound is:

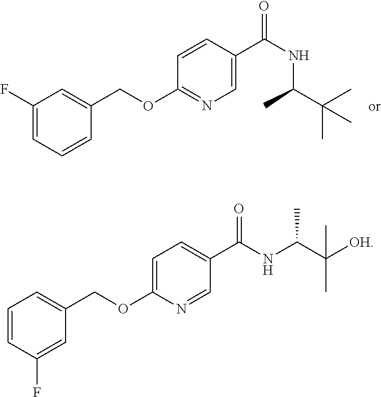

In a further aspect, the compound is:

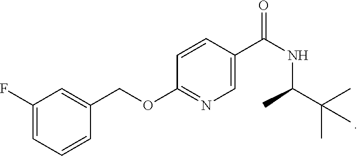

In a further aspect, the compound is:

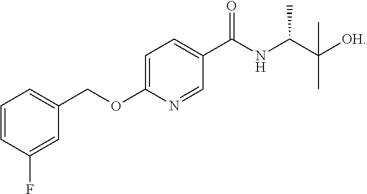

It is contemplated that one or more compounds can optionally be omitted from the disclosed invention.

### D. Metabotropic Glutamate Receptor Activity

The utility of the compounds in accordance with the present invention as potentiators of metabotropic glutamate receptor activity, in particular mGluR5 activity, can be demonstrated by methodology known in the art. Human embryonic kidney (HEK) cells transfected with rat mGluR5 were plated in clear bottom assay plates for assay in a Functional Drug Screening System (FDSS). The cells were loaded with a Ca²⁺-sensitive fluorescent dye (e.g., Fluo-4), and the plates were washed and placed in the FDSS instrument. After establishment of a fluorescence baseline for twelve seconds, the compounds of the present invention were added to the cells, and the response in cells was measured. Five minutes later, an mGluR5 agonist (e.g., glutamate, 3,5-dihydroxyphenylglycine, or quisqualate) was added to the cells, and the response of the cells was measured. Potentiation of the agonist response of mGluR5 by the compounds in the present invention was observed as an increase in response to non-maximal concentrations of agonist (here, glutamate) in the presence of compound compared to the response to agonist in the absence of compound.

The above described assay operated in two modes. In the first mode, a range of concentrations of the present compounds were added to cells, followed by a single fixed concentration of agonist. If a compound acted as a potentiator, an EC₅₀ value for potentiation and a maximum extent of potentiation by the compound at this concentration of agonist was determined by non-linear curve fitting. In the second mode, several fixed concentrations of the present compounds were added to various wells on a plate, followed by a range of concentrations of agonist for each concentration of present compound; the EC₅₀ values for the agonist at each concentration of compound were determined by non-linear curve fitting. A decrease in the EC₅₀ value of the agonist with increasing concentrations of the present compounds (a leftward shift of the agonist concentration-response curve) is an indication of the degree of mGluR5 potentiation at a given concentration of the present compound. An increase in the EC₅₀ value of the agonist with increasing concentrations of the present compounds (a rightward shift of the agonist concentration-response curve) is an indication of the degree of mGluR5 antagonism at a given concentration of the present compound. The second mode also indicates whether the present compounds also affect the maximum response to mGluR5 to agonists.

In particular, the disclosed compounds had activity in potentiating the mGluR5 receptor in the aforementioned assays, generally with an EC₅₀ for potentiation of less than about 10 μM. Preferred compounds within the present invention had activity in potentiating the mGluR5 receptor with an EC₅₀ for potentiation of less than about 500 nM. Preferred compounds further caused a leftward shift of the agonist EC₅₀ by greater than 3-fold. These compounds did not cause mGluR5 to respond in the absence of agonist, and they did not elicit a significant increase in the maximal response of mGluR5 to agonists. These compounds are positive allosteric modulators (potentiators) of human and rat mGluR5 and were selective for mGluR5 compared to the other seven subtypes of metabotropic glutamate receptors.

In vivo efficacy for disclosed compounds can be measured in a number of preclinical rat behavioral model where known, clinically useful antipsychotics display similar positive responses. For example, disclosed compounds can reverse amphetamine-induced hyperlocomotion in male Sprague-Dawley rats at doses ranging from 1 to 100 mg/kg i.p.

### E. Methods of Making the Compounds

In one aspect, the invention relates to methods of making compounds useful as positive allosteric modulators (potentiators) of the metabotropic glutamate receptor subtype 5 (mGluR5), which can beuseful in the treatment neurological and psychiatric disorders associated with glutamate dysfunction and other diseases in which metabotropic glutamate receptors are involved.

The compounds of this invention can be prepared by employing reactions as shown in the following schemes, in addition to other standard manipulations that are known in the literature, exemplified in the experimental sections or clear to one skilled in the art. Substituent numbering as shown in schemes does not necessarily correlate to that used in the claims and often, for clarity, a single substituent is shown to attach to the compound where multiple substituents are allowed under the definitions disclosed herein.

Reactions used to generate the compounds of this invention are prepared by employing reactions as shown in the following Reaction Schemes, in addition to other standard manipulations known in the literature or to one skilled in the art. The following examples are provided so that the invention might be more fully understood, are illustrative only, and should not be construed as limiting.

1. Reaction Scheme I

In one aspect, disclosed compounds can be prepared as shown below.

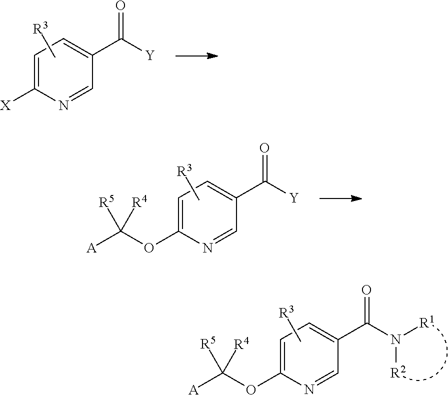

Compounds are represented in generic form, with substituents as noted in compound descriptions elsewhere herein. A more specific example is set forth below.

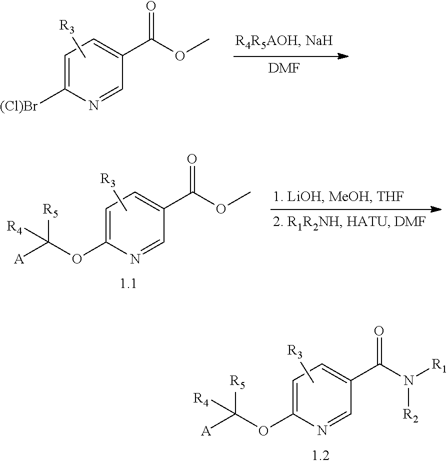

Examples of ethers of type 1.2 can be prepared as outlined in Scheme 1. Starting from 6-halogenated nicotinates displacement using various alcohols provides ester intermediates of type 1.1 which upon saponification and subsequent amide coupling gives Examples 1.2.

In one aspect, Scheme 1 involves S_{N}Ar reaction of a halonicotinic ester or acid with an appropriate alcohol. It is contemplated that alternative leaving groups can be employed. It is also contemplated that base can also be employed to increase the nucleophilicity of the alcohol (i.e., provide an alkoxide). In a further aspect, Scheme 1 also involves reaction of the resulting O-substituted compound with an appropriate amine, thereby providing an amide. Specific reactions conditions for various examples are also provided herein.

2. Reaction Scheme II

In one aspect, disclosed compounds can be prepared as shown below.

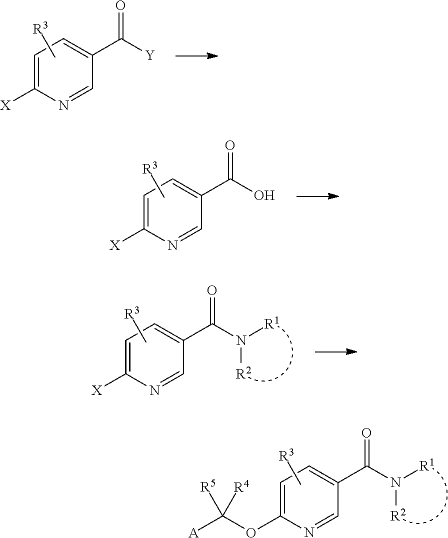

Compounds are represented in generic form, with substituents as noted in compound descriptions elsewhere herein. A more specific example is set forth below.

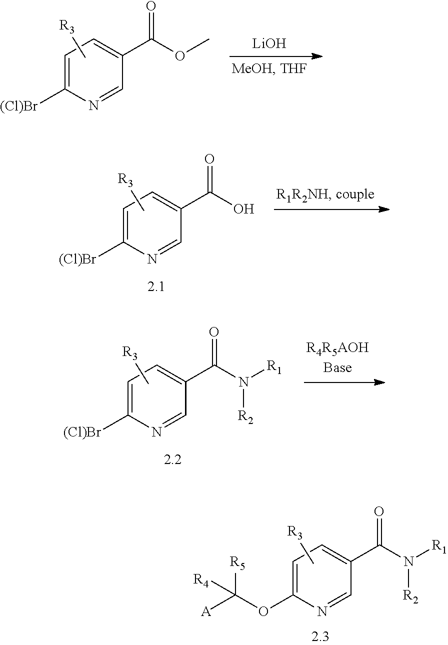

Alternatively nicotinamide examples can be prepared according to Scheme 2, wherein the starting ester is first hydrolyzed to acid 2.1, coupled to give Intermediate 2.2 and under basic conditions with or without an optional copper salt a displacement reaction can occur with an appropriate alcohol to give final Examples 2.3.

In one aspect, Scheme 2 involves the same basic transformations as used in Scheme 1, but employs a different reaction order. Specific reactions conditions for various examples are also provided herein.

3. Reaction Scheme III

In one aspect, disclosed compounds can be prepared as shown below.

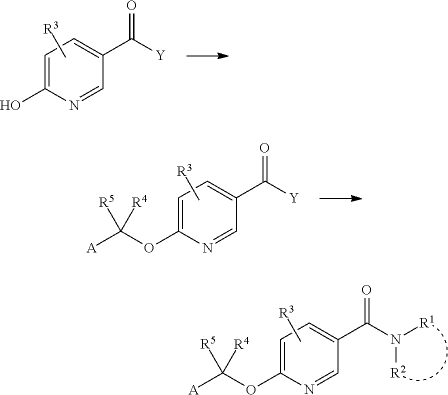

Compounds are represented in generic form, with substituents as noted in compound descriptions elsewhere herein. A more specific example is set forth below.

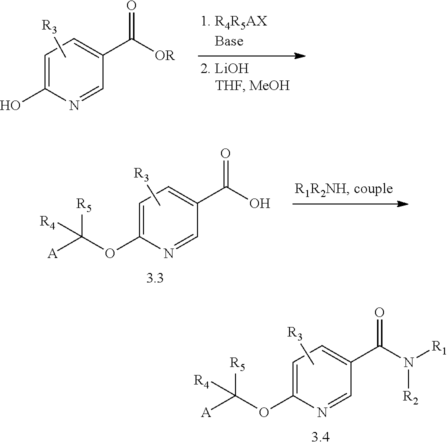

In one aspect, Scheme 3 involves reaction of an alkyl 6-hydroxynicotinate with an appropriate alkyl halide, optionally in the presence of a suitable base to form the more nucleophilic phenoxides analog. It is contemplated that alternative leaving groups can be employed. In a further aspect, Scheme 3 also involves reaction of the resulting O-substituted compound with an appropriate amine, thereby providing an amide. Specific reactions conditions for various examples are also provided herein.

4. Reaction Scheme IV

In one aspect, disclosed compounds can be prepared as shown below.

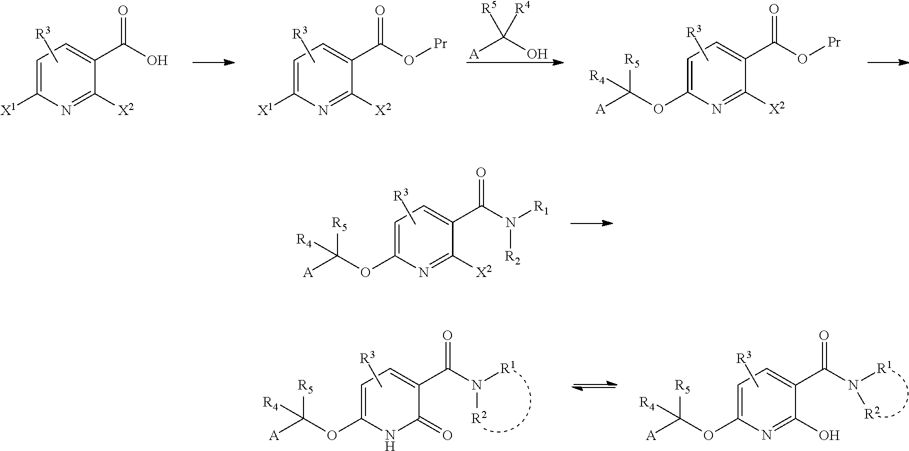

Compounds are represented in generic form, with substituents as noted in compound descriptions elsewhere herein. A more specific example is set forth below.

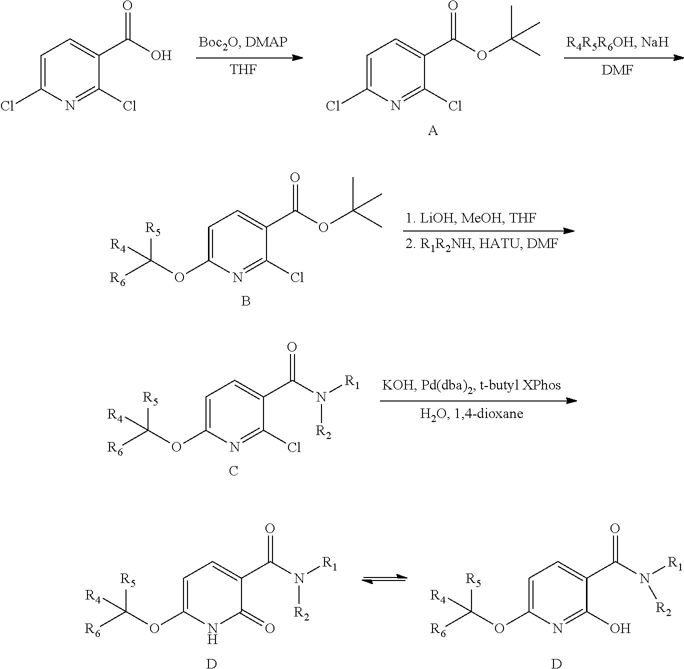

In one aspect, Scheme 4 involves protection of an optionally substituted 2,6-dihalonicotinic acid. The group Pr represents a protecting group, for example, a tert-butyl group. Aromatic nucleophilic substitution with an appropriate alcohol, optionally in the presence of a suitable base to form the more nucleophilic alkoxide analog, can provide on ether. It is contemplated that alternative leaving groups can be employed. In a further aspect, Scheme 4 also involves, after deprotection, reaction of the resulting compound with an appropriate amine, thereby providing an amide. Specific reactions conditions for various examples are also provided herein.

Thus, in one aspect, the invention relates to a method of making a compound, or pharmaceutically acceptable salt or N-oxide thereof, comprising the step of reacting a first compound having a structure represented by a formula:

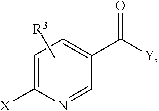

wherein X is halogen; wherein Y is −OR⁶ or —NR¹R²; wherein R⁶ is alkyl or aryl; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; and wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; with a second compound having a structure represented by a formula:

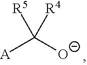

wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and wherein A is an optionally substituted cyclic organic residue selected from aryl, heteroaryl, cycloalkyl, heterocycloalkyl, cycloalkenyl, and heterocycloalkenyl, thereby providing a compound having a structure represented by a formula:

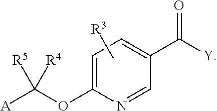

In a further aspect, the first compound has a structure represented by a formula:

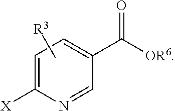

In a further aspect, R³ is 0-1 non-hydrogen substituents independently selected from C1 to C4 alkyl, C1 to C4 haloalkyl, halide, hydroxyl, trifluoromethyl, cyano, nitro, azide, C1 to C4 alkoxy, thiol, C1 to C4 alkylsulfonyl, C1 to C4 carboxamide or C1 to C4 sulfonamide.

In a further aspect, the first compound has a structure represented by a formula:

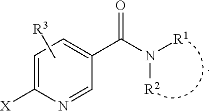

In a further aspect, Y is NR¹H and R¹ is a C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide. In a further aspect, X is Br or Cl. In a further aspect, R⁶ is alkyl selected from methyl, ethyl, propyl, butyl, pentyl, or hexyl.

In a further aspect, reacting is a nucleophilic substitution reaction in the presence of sodium hydride.

In a further aspect, R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and wherein A is an optionally substituted C3 to C9 cyclic organic residue selected from aryl, heteroaryl, cycloalkyl, heterocycloalkyl, cycloalkenyl, and heterocycloalkenyl.

In a further aspect, the compound provided has a structure represented by a formula:

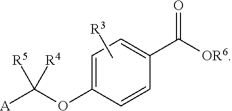

In a further aspect, the compound provided has a structure represented by a formula:

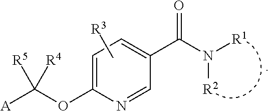

In a further aspect, the compound provided has a structure represented by a formula:

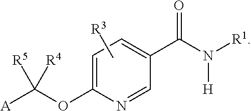

In one aspect, the invention relates to a method of making a compound, or pharmaceutically acceptable salt or N-oxide thereof, comprising the step of reacting a first compound having a structure represented by a formula:

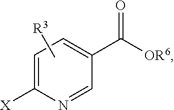

wherein X is halogen or

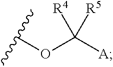

wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl, wherein R⁶ is alkyl or aryl; and wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; with a second compound having a structure represented by a formula:

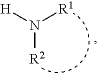

wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons, thereby providing a compound having a structure represented by a formula:

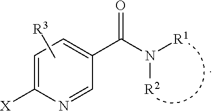

In a further aspect, X is halogen selected from Br and Cl.

In a further aspect, the first compound has a structure represented by a formula:

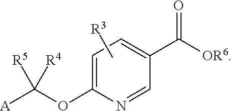

In a further aspect, R³ is 0-1 non-hydrogen substituents independently selected from C1 to C4 alkyl, C1 to C4 haloalkyl, halide, hydroxyl, trifluoromethyl, cyano, nitro, azide, C1 to C4 alkoxy, thiol, C1 to C4 alkylsulfonyl, C1 to C4 carboxamide or C1 to C4 sulfonamide. In a further aspect, R⁶ is alkyl selected from methyl, ethyl, propyl, butyl, pentyl, or hexyl.

In a further aspect, reacting is hydrolysis in the presence of LiOH, followed by an amidation reaction in the presence of a coupling reagent. In a further aspect, the coupling reagent is 2-(7-aza-1H-benzotriazole-1-yl)-1,1,3,3-tetramethyluronium hexafluorophosphate.

In a further aspect, R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and wherein A is an optionally substituted C3 to C9 cyclic organic residue selected from aryl, heteroaryl, cycloalkyl, heterocycloalkyl, cycloalkenyl, and heterocycloalkenyl.

In a further aspect, the compound provided has a structure represented by a formula:

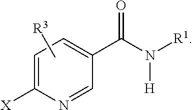

In a further aspect, the compound provided has a structure represented by a formula:

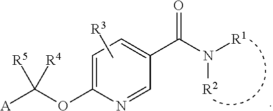

In a further aspect, the compound provided has a structure represented by a formula:

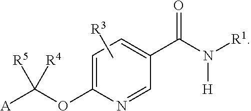

In a further aspect, the method provides a disclosed compound, for example, a compound listed in Tables 1 and 2. Compounds in the Tables were synthesized as disclosed herein. The requisite starting materials were commercially available, described in the literature or readily synthesized by one skilled in the art of organic synthesis.

TABLE 1

| | | | | EC₅₀ |
|---|---|---|---|---|
| Example | Structure/Name | MW | M + H | (nM) |
| 1.2a.4 | 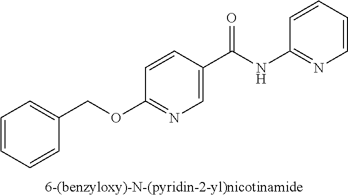 | 305.1 | 306.1 | 410 |
| 1.2a.5 | 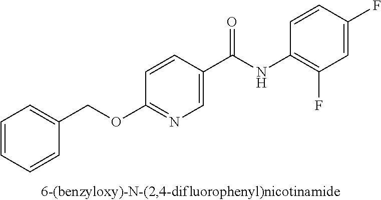 | 340.1 | 341.1 | 140 |
| 1.2a.6 | 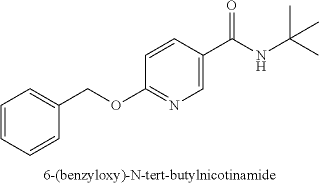 | 284.1 | 285.1 | 230 |
| 1.2a.7 | 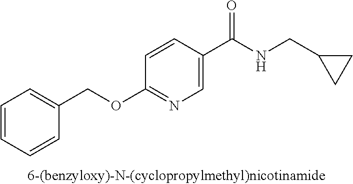 | 282.1 | 283.1 | 450 |
| 1.2a.8 | 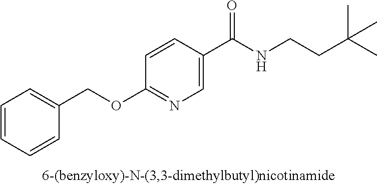 | 312.1 | 313.2 | 92 |
| 1.2a.9 | 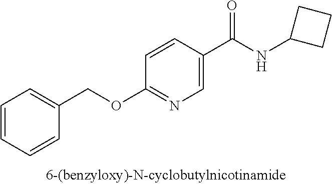 | 282.1 | 283.1 | 340 |
| 1.2a.10 | 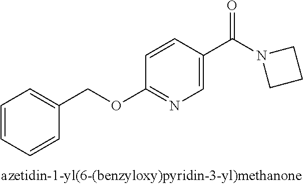 | 268.1 | 269.1 | 3700 |
| 1.2a.11 | 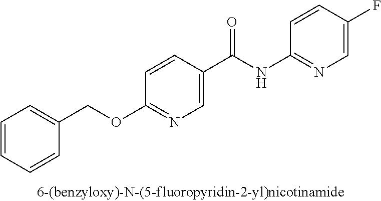 | 323.1 | 324.1 | 280 |
| 1.2a.12 | 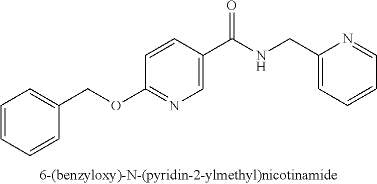 | 319.1 | 320.1 | 3400 |
| 1.2a.13 | 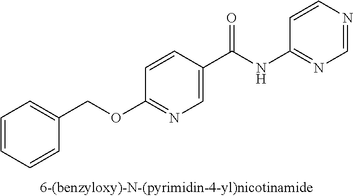 | 306.1 | 307.1 | 1100 |
| 1.2a.14 | 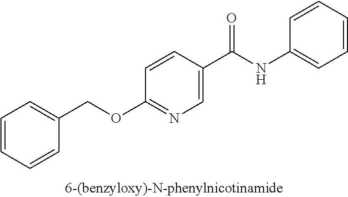 | 304.1 | 305.1 | 380 |
| 1.2a.15 | 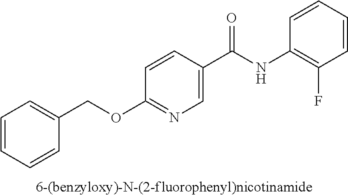 | 322.1 | 323.1 | 310 |
| 1.2a.16 | 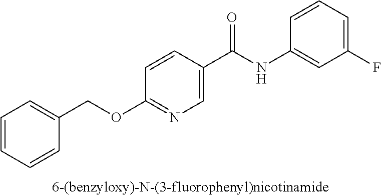 | 322.1 | 323.1 | 530 |
| 1.2a.17 | 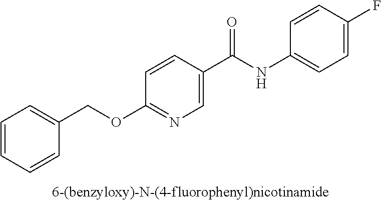 | 322.1 | 323.1 | 340 |
| 1.2a.18 | 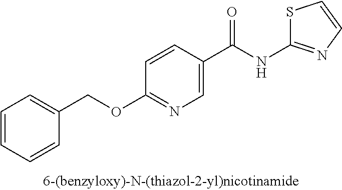 | 311.0 | 312.1 | 520 |
| 1.2a.19 | 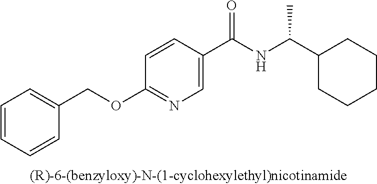 | 338.2 | 339.2 | 40 |
| 1.2a.20 | 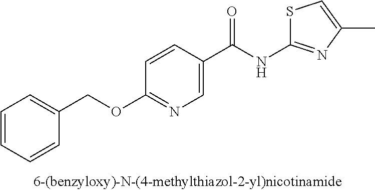 | 325.0 | 326.1 | 1600 |
| 1.2a.21 | 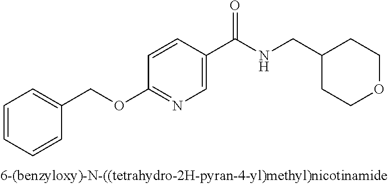 | 326.1 | 327.2 | 3900 |
| 1.2a.22 | 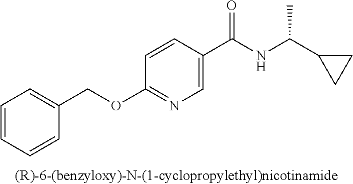 | 296.1 | 297.2 | 170 |
| 1.2a.23 | 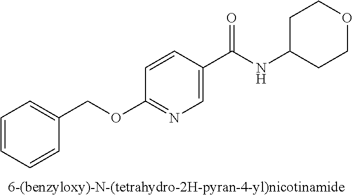 | 312.1 | 313.2 | 3900 |
| 1.2a.24 | 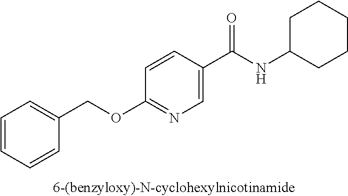 | 310.3 | 311.4 | 140 |
| 1.2d.1 | 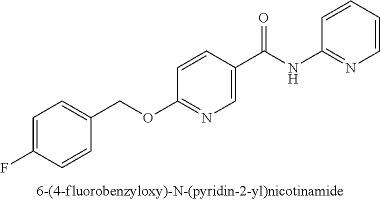 | 323.1 | 324.1 | 1300 |
| 1.2d.2 | 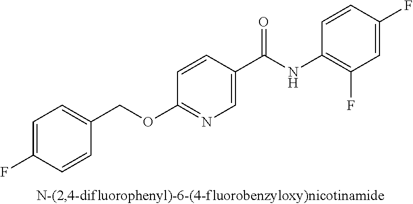 | 358.0 | 359.1 | 210 |
| 1.2d.3 | 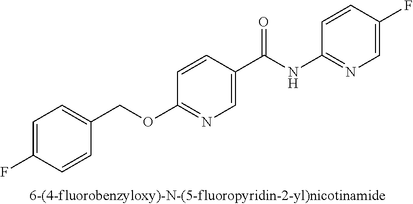 | 341.1 | 342.1 | 510 |
| 1.2d.4 | 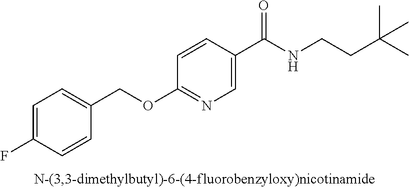 | 330.1 | 331.2 | 690 |
| 1.2b.2 | 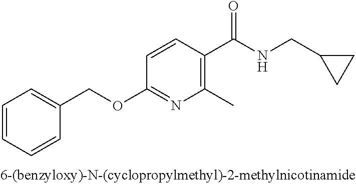 | 296.1 | 297.2 | 1600 |
| 1.2b.3 | 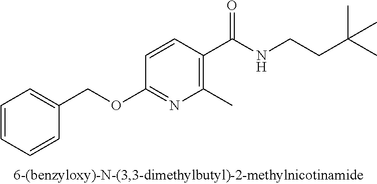 | 326.2 | 327.2 | 400 |
| 1.2c.3 | 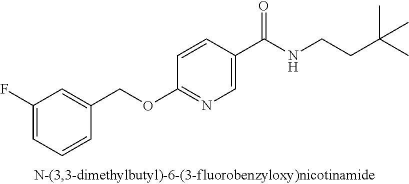 | 330.1 | 331.2 | 85 |
| 1.2c.4 | 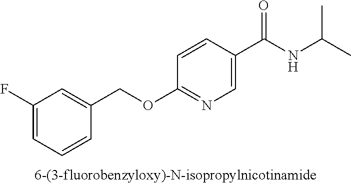 | 288.1 | 289.1 | 1000 |
| 1.2c.5 | 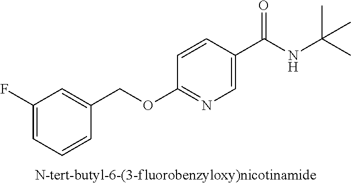 | 302.1 | 303.2 | 400 |
| 1.2c.6 | 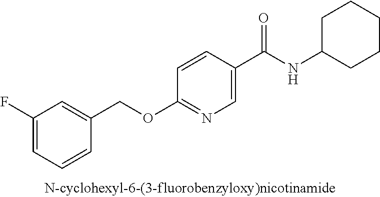 | 328.1 | 329.2 | 40 |
| 1.2c.7 | 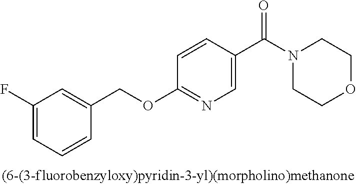 | 316.1 | 317.1 | 1700 |
| 1.2c.8 | 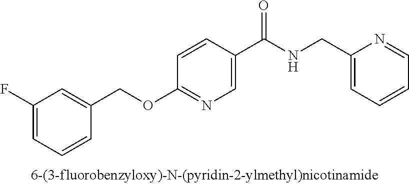 | 337.1 | 338.1 | 2700 |
| 1.2c.9 | 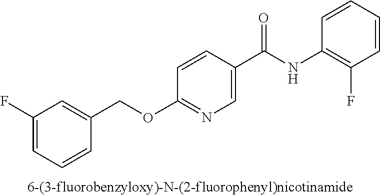 | 340.1 | 341.1 | 210 |
| 1.2c.10 | 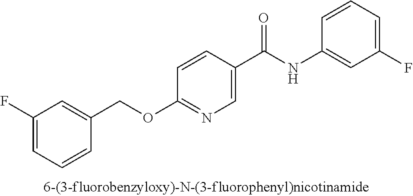 | 340.1 | 341.1 | 460 |
| 1.2c.11 | 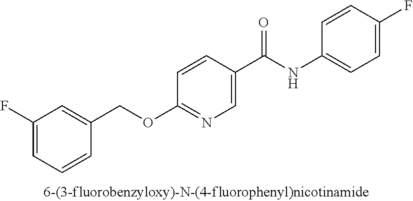 | 340.1 | 341.1 | 260 |
| 1.2c.12 | 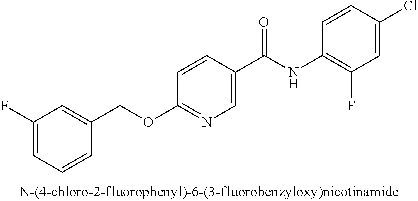 | 374.0 | 375.1 | 570 |
| 1.2c.13 | 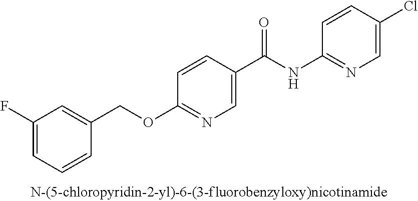 | 357.0 | 358.1 | 346 |
| 1.2c.14 | 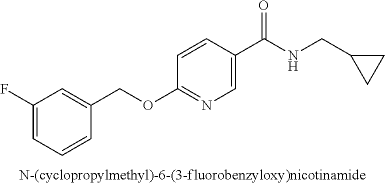 | 300.1 | 301.1 | 920 |
| 1.2c.15 | 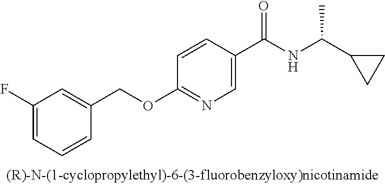 | 314.1 | 315.2 | 94 |
| 1.2c.16 | 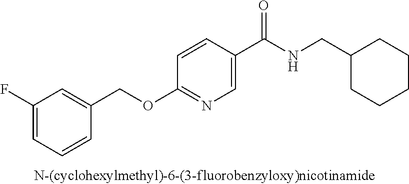 | 342.1 | 343.2 | 230 |
| 1.2c.17 | 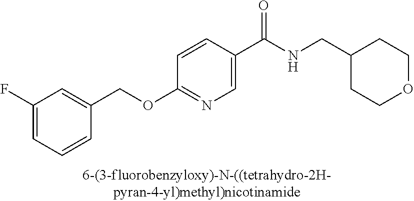 | 344.1 | 345.2 | 1100 |
| 1.2c.18 | 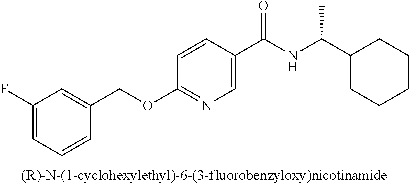 | 356.1 | 357.2 | 190 |
| 1.2c.19 | 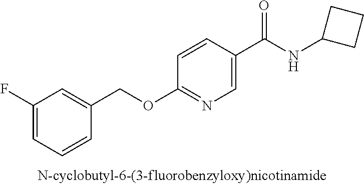 | 300.3 | 300.4 | 120 |
| 1.2c.20 | 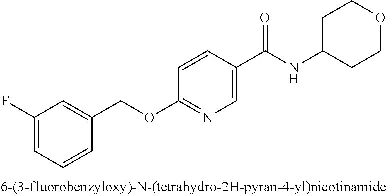 | 330.1 | 331.2 | 1600 |
| 1.2c.21 | 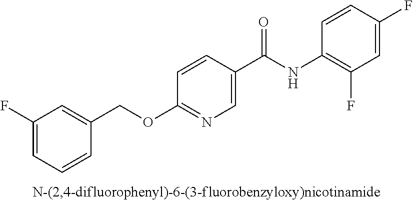 | 358.3 | 359.4 | 76 |
| 1.2e.1 | 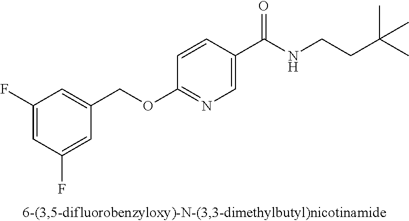 | 348.1 | 349.2 | 1100 |
| 1.2f.1 | 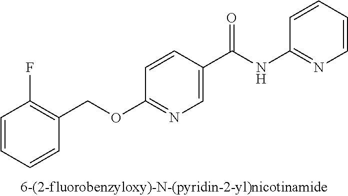 | 323.1 | 324.1 | 300 |
| 1.2f.2 | 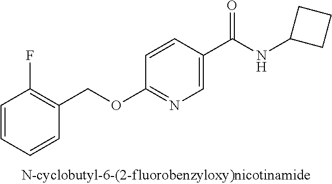 | 300.3 | 301.4 | 270 |
| 1.2f.3 | 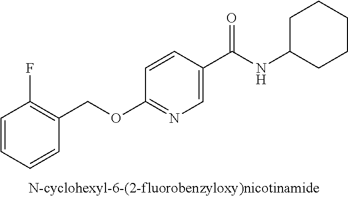 | 328.3 | 329.3 | 110 |
| 1.2g.1 | 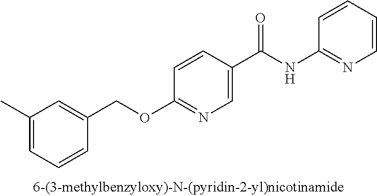 | 319.1 | 320.1 | 240 |
| 1.2g.2 | 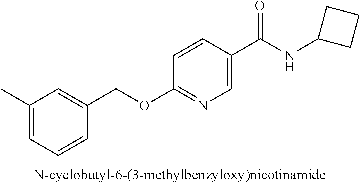 | 296.3 | 297.4 | 120 |
| 1.2g.3 | 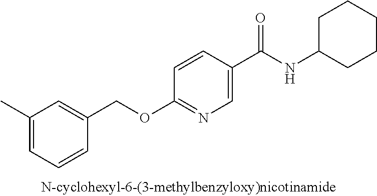 | 324.4 | 325.4 | 100 |
| 1.2h.1 | 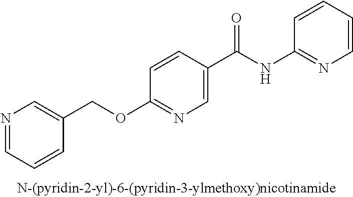 | 306.1 | 307.1 | 2900 |
| 1.2i.2 | 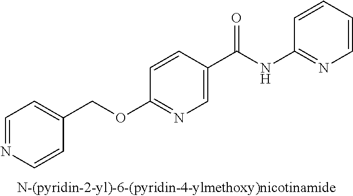 | 306.1 | 307.1 | 10000 |
| 1.2i.3 | 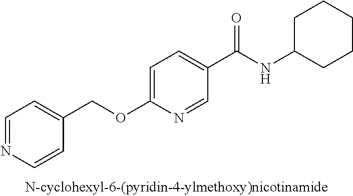 | 311.3 | 312.4 | 1200 |
| 1.2i.4 | 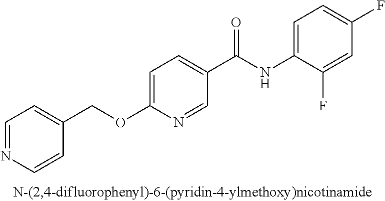 | 341.3 | 342.3 | 810 |
| 1.2i.5 | 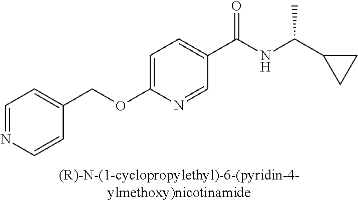 | 297.3 | 298.4 | 4800 |
| 1.2j.2 | 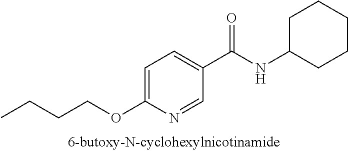 | 276.3 | 277.4 | 2400 |
| 1.2j.3 | 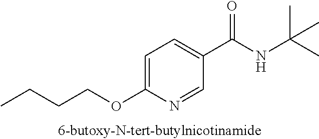 | 250.3 | 251.3 | 10000 |
| 1.2j.4 | 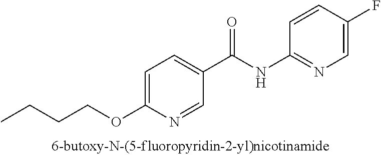 | 289.3 | 290.4 | 1500 |
| 1.2j.5 | 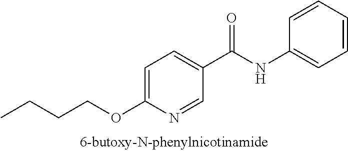 | 270.3 | 271.3 | 650 |
| 1.2j.6 | 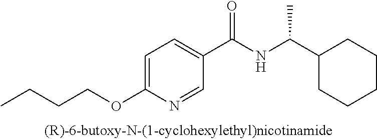 | 304.4 | 305.4 | 1500 |
| 1.2j.7 | 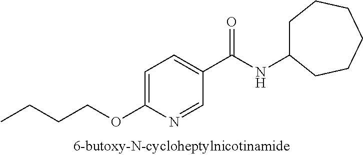 | 290.4 | 291.4 | 2200 |
| 1.2m.1 | 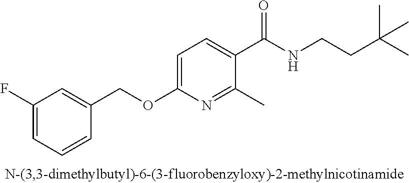 | 344.4 | 345.5 | 1000 |
| 1.2m.2 | 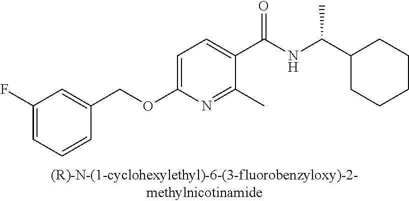 | 370.4 | 371.5 | 870 |
| 1.2m.3 | 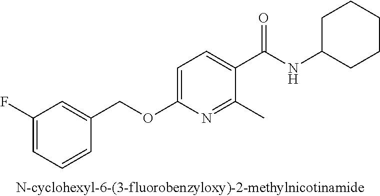 | 342.4 | 343.4 | 610 |
| 1.2n.1 | 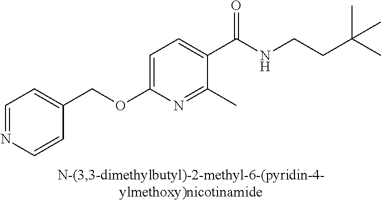 | 327.4 | 328.4 | 3400 |
| 1.2n.2 | 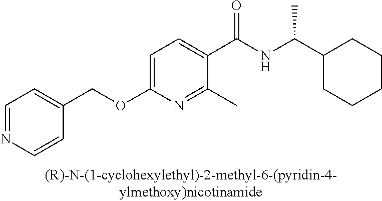 | 353.4 | 354.5 | 800 |
| 2.3a.4 | 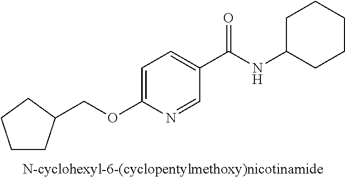 | 302.4 | 303.4 | 710 |
| 2.3a.6 | 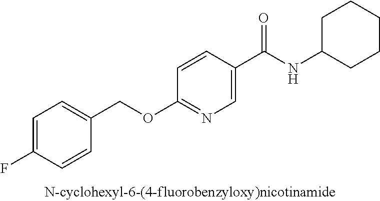 | 328.3 | 329.4 | 750 |
| 2.3a.7 | 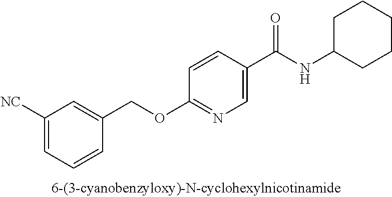 | 335.4 | 336.4 | 95 |
| 2.3a.8 | 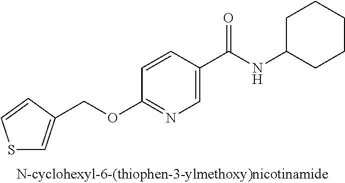 | 316.4 | 317.4 | 120 |
| 2.3a.9 | 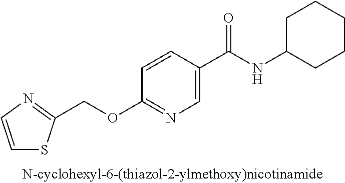 | 317.4 | 318.4 | 3100 |
| 2.3b.1 | 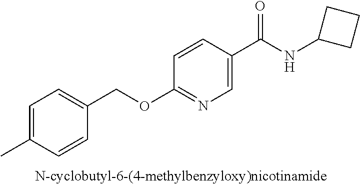 | 296.3 | 297.4 | 1800 |
| 2.3b.2 | 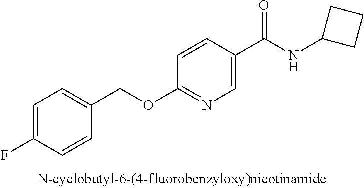 | 300.3 | 300.4 | 1200 |
| 2.3c.1 | 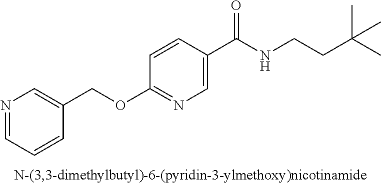 | 313.3 | 314.4 | 2900 |
| 2.3c.2 | 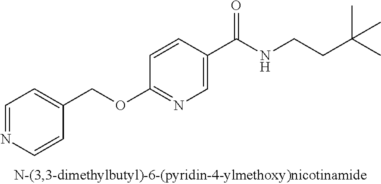 | 313.4 | 314.4 | 420 |

TABLE 2

| | | | EC₅₀ |
|---|---|---|---|
| Structure | MW | M + H | (nM) |
| 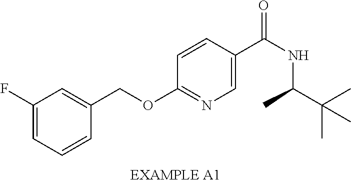 | 330.4 | 331 | 2.2 |
| 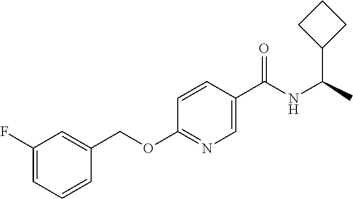 | 328.4 | 329 | 6.6 |
| 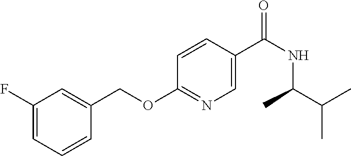 | 316.4 | 317 | 21 |
| 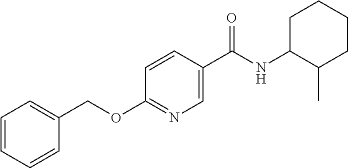 | 324.4 | 325 | 36 |
| 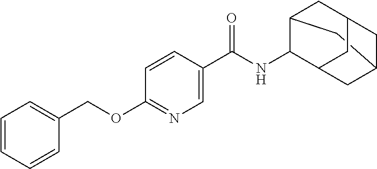 | 362.5 | 363 | 39 |
| 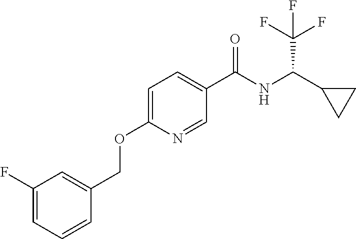 | 368.3 | 369 | 100 |
| 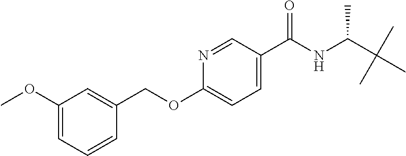 | 342.4 | 343 | 120 |
| 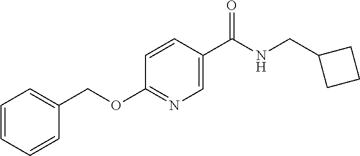 | 296.4 | 297 | 130 |
| 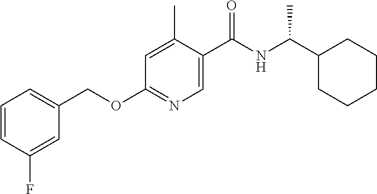 | 370.5 | 371 | 130 |
| 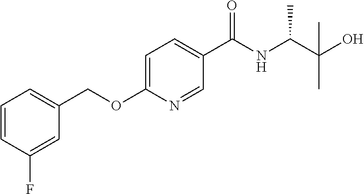 | 332.4 | 333 | 150 |
| 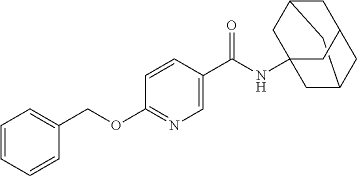 | 362.5 | 363 | 180 |
| 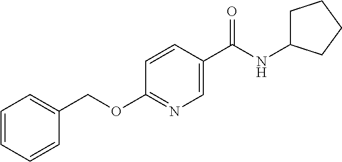 | 296.4 | 297 | 180 |
| 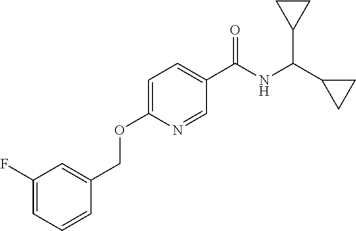 | 340.4 | 341 | 300 |
| 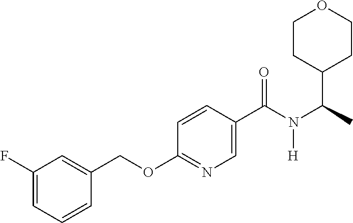 | 358.4 | 359 | 310 |
| 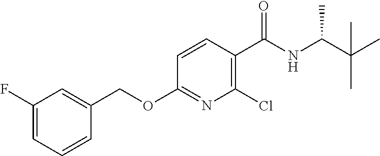 | 364.8 | 366 | 370 |
| 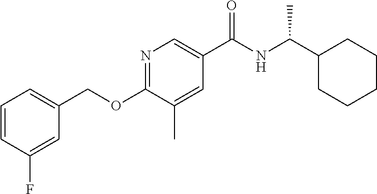 | 370.5 | 371 | 490 |
| 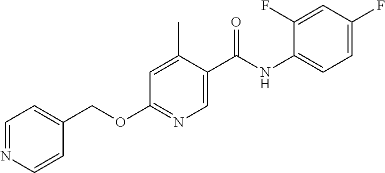 | 355.3 | 356 | 490 |
| 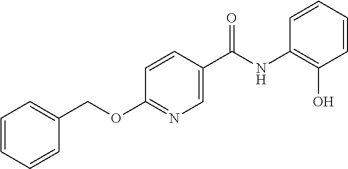 | 320.3 | 321 | 520 |
| 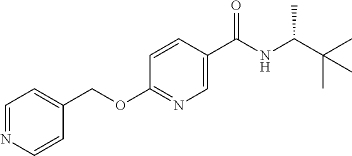 | 313.4 | 314 | 570 |
| 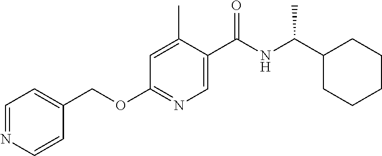 | 353.5 | 354 | 610 |
| 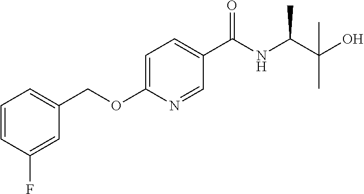 | 332.4 | 333 | 610 |
| 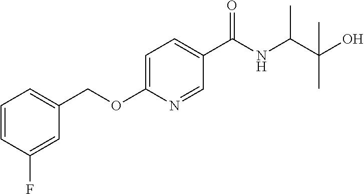 | 332.4 | 333 | 660 |
| 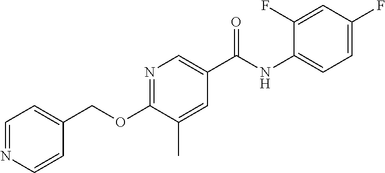 | 355.3 | 356 | 680 |
| 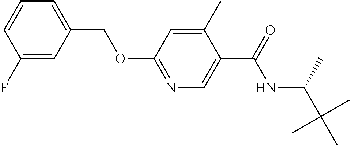 | 344.4 | 345 | 690 |
| 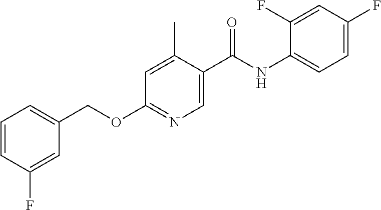 | 372.3 | 373 | 920 |
| 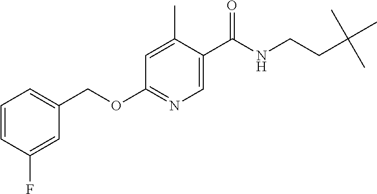 | 344.4 | 345 | 960 |
| 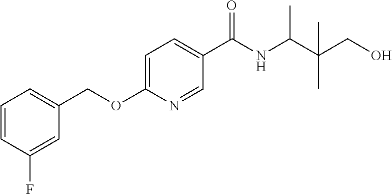 | 346.4 | 347 | 1,000 |
| 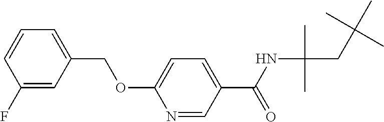 | 358.5 | 359 | 1,100 |
| 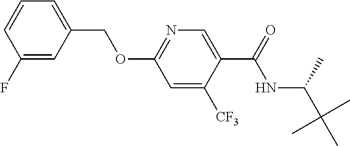 | 398.4 | 399 | 1,200 |
| 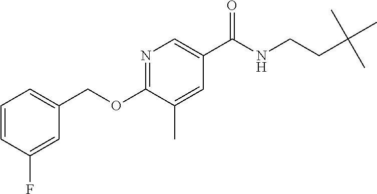 | 344.4 | 345 | 1,300 |
| 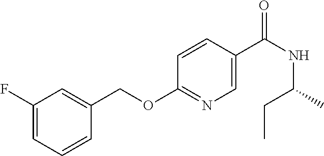 | 302.3 | 303 | 1,300 |
| 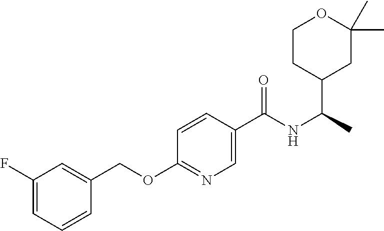 | 386.5 | 387 | 1,700 |
| 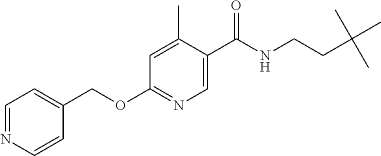 | 327.4 | 328 | 1,700 |
| 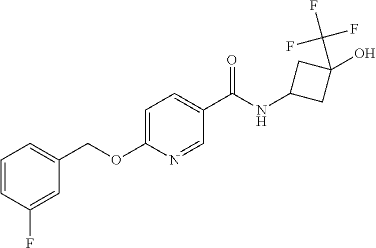 | 384.3 | 385 | 1,800 |
| 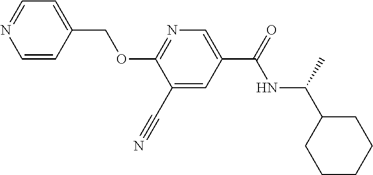 | 364.4 | 365 | 1,900 |
| 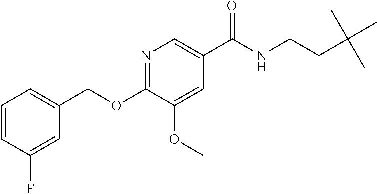 | 360.4 | 361 | 1,900 |
| 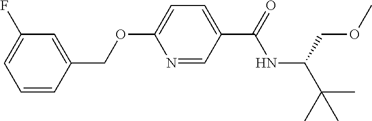 | 360.4 | 361 | 1,900 |
| 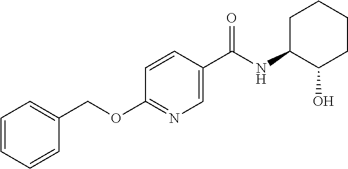 | 326.4 | 327 | 2,800 |
| 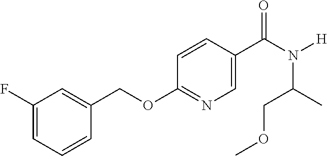 | 318.3 | 319 | 3,000 |
| 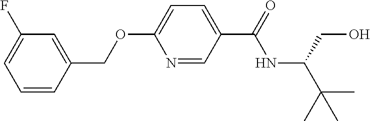 | 346.4 | 347 | 3,200 |
| 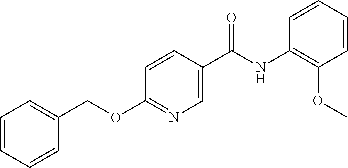 | 334.4 | 335 | 3,400 |
| 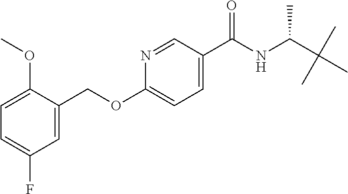 | 360.4 | 361 | 3,500 |
| 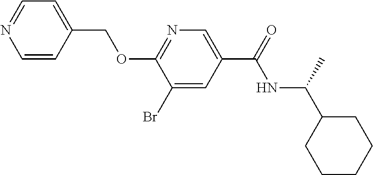 | 418.3 | 419 | 3,600 |
| 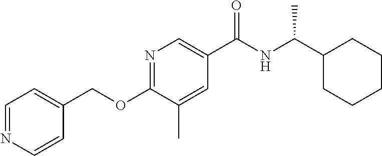 | 353.5 | 354 | 3,600 |
| 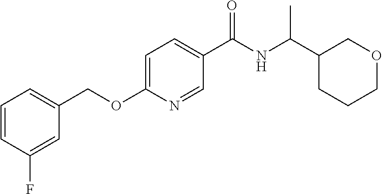 | 358.4 | 359 | 3,700 |
| 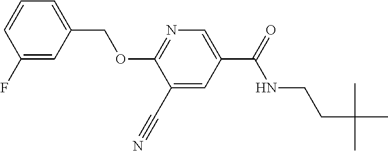 | 355.4 | 356 | 3,900 |
| 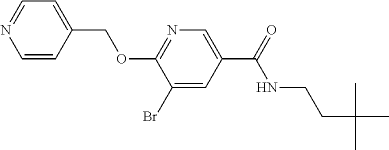 | 392.3 | 393 | 3,900 |
| 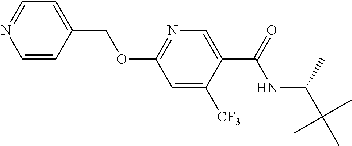 | 381.4 | 382 | 3,900 |
| 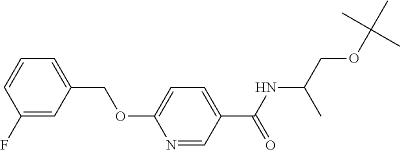 | 360.4 | 361 | 4,200 |
| 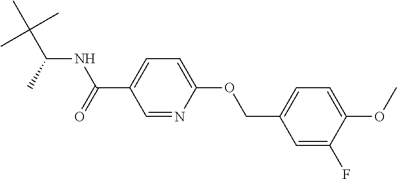 | 360.4 | 361 | 4,600 |
| 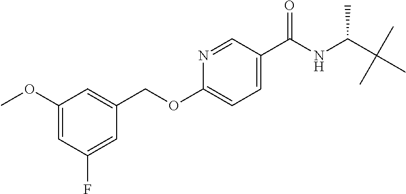 | 360.4 | 361 | 5,100 |
| 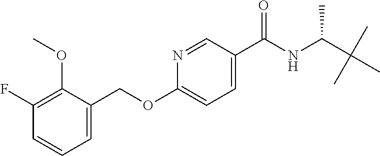 | 360.4 | 361 | 5,200 |
| 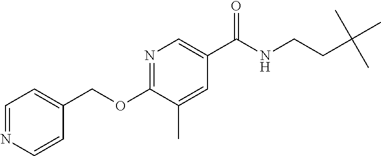 | 327.4 | 328 | 5,300 |
| 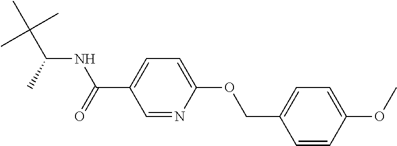 | 342.4 | 343 | 5,400 |
| 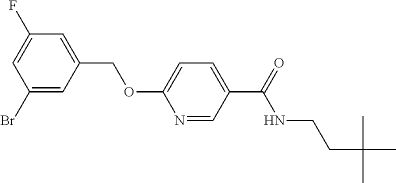 | 409.3 | 410 | >10,000 |
| 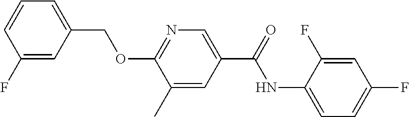 | 372.3 | 373 | >10,000 |
| 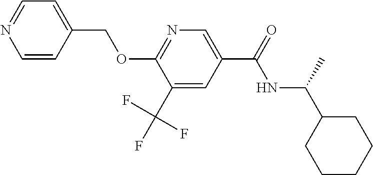 | 407.4 | 408 | >10,000 |
| 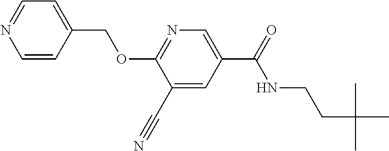 | 338.4 | 339 | >10,000 |
| 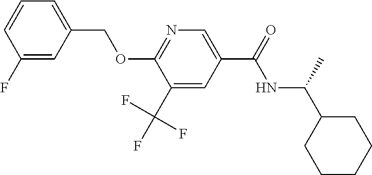 | 424.4 | 425 | >10,000 |
| 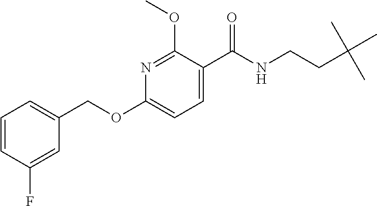 | 360.4 | 361 | >10,000 |
| 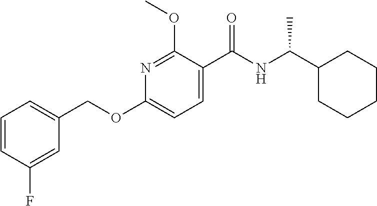 | 386.5 | 387 | >10,000 |
| 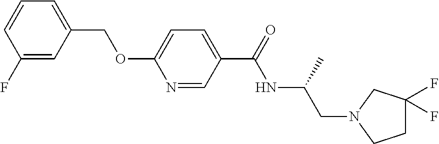 | 393.4 | 394 | >10,000 |
| 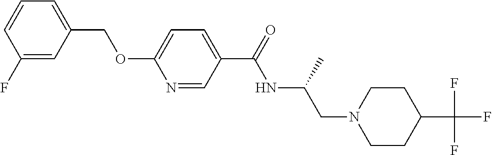 | 439.5 | 440 | >10,000 |
| 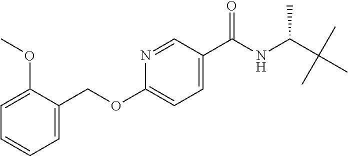 | 342.4 | 343 | >10,000 |
| 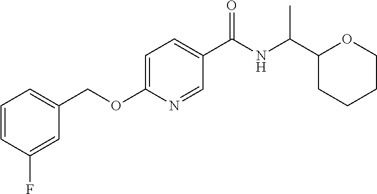 | 358.4 | 359 | >10,000 |
| 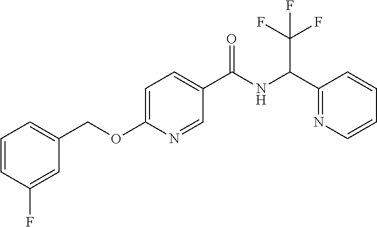 | 405.4 | 406 | >10,000 |
| 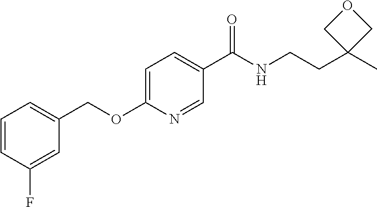 | 344.4 | 345 | >10,000 |
| 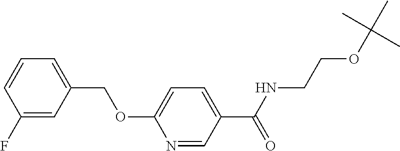 | 346.4 | 347 | >10,000 |
| 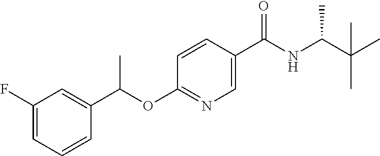 | 344.4 | 345 | >10,000 |
| 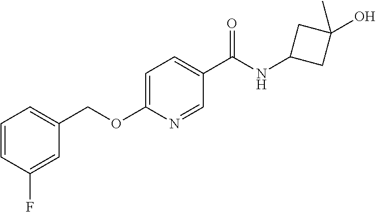 | 330.4 | 331 | >10,000 |
| 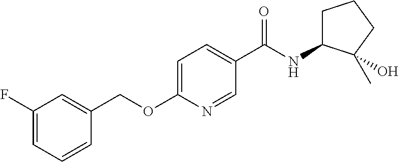 | 344.4 | 345 | >10,000 |
| 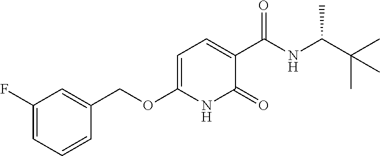 | 346.4 | 347 | >10,000 |
| 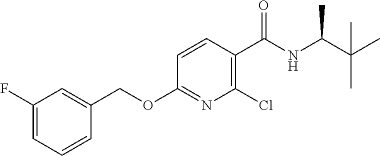 | 364.8 | 366 | >10,000 |

Thus, it is understood that a disclosed methods can be used to provide the disclosed compounds.

F. Pharmaceutical Compositions

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

A potentiated amount of an mGluR agonist to be administered in combination with an effective amount of a disclosed compound can be expected to vary from about 0.1 milligram per kilogram of body weight per day (mg/kg/day) to about 100 mg/kg/day and is expected to be less than the amount that is required to provide the same effect when administered without an effective amount of a disclosed compound. Preferred amounts of a co-administered mGluR agonist are able to be determined by one skilled in the art.

In the treatment conditions which require potentiation of metabotropic glutamate receptor activity an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the from of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The disclosed pharmaceutical compositions can further comprise other therapeutically active compounds, which are usually applied in the treatment of the above mentioned pathological conditions.

It is understood that the disclosed compositions can be employed in the disclosed methods of using.

G. Methods of Using the Compounds and Compositions

The amino acid L-glutamate (referred to herein simply as glutamate) is the principal excitatory neurotransmitter in the mammalian central nervous system (CNS). Within the CNS, glutamate plays a key role in synaptic plasticity (e.g., long term potentiation (the basis of learning and memory)), motor control and sensory perception. It is now well understood that a variety of neurological and psychiatric disorders, including, but not limited to, schizophrenia general psychosis and cognitive deficits, are associated with dysfunctions in the glutamatergic system. Thus, modulation of the glutamatergic system is an important therapeutic goal. Glutamate acts through two distinct receptors: ionotropic and metabotropic glutamate receptors. The first class, the ionotropic glutamate receptors, is comprised of multi-subunit ligand-gated ion channels that mediate excitatory post-synaptic currents. Three subtypes of ionotropic glutamate receptors have been identified, and despite glutamate serving as agonist for all three receptor subtypes, selective ligands have been discovered that activate each subtype. The ionotropic glutamate receptors are named after their respective selective ligands: kainite receptors, AMPA receptors and NMDA receptors.

The second class of glutamate receptor, termed metabotropic glutamate receptors, (mGluRs), are G-protein coupled receptors (GPCRs) that modulate neurotransmitter release or the strength of synaptic transmission, based on their location (pre- or post-synaptic). The mGluRs are family C GPCR, characterized by a large (˜560 amino acid) “venus fly trap” agonist binding domain in the amino-terminal domain of the receptor. This unique agonist binding domain distinguishes family C GPCRs from family A and B GPCRs wherein the agonist binding domains are located within the 7-strand transmembrane spanning (7™) region or within the extracellular loops that connect the strands to this region. To date, eight distinct mGluRs have been identified, cloned and sequenced. Based on structural similarity, primary coupling to intracellular signaling pathways and pharmacology, the mGluRs have been assigned to three groups: Group I (mGluR1 and mGluR5), Group II (mGluR2 and mGluR3) and Group III (mGluR4, mGluR6, mGluR7 and mGluR8). Group I mGluRs are coupled through Gαq/11 to increase inositol phosphate and metabolism and resultant increases in intracellular calcium. Group I mGluRs are primarily located post-synaptically and have a modualtory effect on ion channel activity and neuronal excitability. Group II (mGluR2 and mGluR3) and Group III (mGluR4, mGluR6, mGluR7 and mGluR8) mGluRs are primarily located pre-synaptically where they regulate the release of neurotransmitters, such as glutamate. Group II and Group III mGluRs are coupled to Gαi and its associated effectors such as adenylate cyclase.

Post-synaptic mGluRs are known to functionally interact with post-synaptic ionotropic glutamate receptors, such as the NMDA receptor. For example, activation of mGluR5 by a selective agonist has been shown to increase post-synaptic NMDA currents (Mannaioni et. al. J. Neurosci. 21:5925-5934 (2001)). Therefore, modulation of mGluRs is an approach to modulating glutamatergic transmission. Numerous reports indicate that mGluR5 plays a role in a number of disease states including anxiety (Spooren et. al. J. Pharmacol. Exp. Therapeut. 295:1267-1275 (2000), Tatarczynska et al. Br. J. Pharmaol. 132:1423-1430 (2001)), schizophrenia (reviewed in Chavez-Noriega et al. Curr. Drug Targets: CNS & Neurological Disorders 1:261-281 (2002), Kinney, G. G. et al. J. Pharmacol. Exp. Therapeut. 313:199-206 (2005)), addiction to cocaine (Chiamulera et al. Nature Neurosci. 4:873-874 (2001), Parkinson's disease (Awad et al. J. Neurosci. 20:7871-7879 (2000), Ossowska et al. Neuropharmacol. 41: 413-420 (2001), and pain (Salt and Binns Neurosci. 100:375-380 (2001).

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

In one aspect, the subject compounds can be coadministered with ant-Alzheimer's agents, beta-secretase inhibitors, gamma-secretase inhibitors, muscarinic agonists, muscarinic potentiatorsHMG-CoA reductase inhibitors, NSAIDs and anti-amyloid antibodies.

In another aspect, the subject compounds can be administered in combination with sedatives, hypnotics, anxiolytics, antipsychotics, selective serotonin reuptake inhibitors (SSRIs), monoamine oxidase inhibitors (MAOIs), 5-HT2 antagonists, GlyT1 inhibitors and the like such as, but not limited to: risperidone, clozapine, haloperidol, fluoxetine, prazepam, xanomeline, lithium, phenobarbitol, and salts thereof and combinations thereof.

In another aspect, the subject compound can be used in combination with levodopa (with or without a selective extracerebral decarboxylase inhibitor), anticholinergics such as biperiden, COMT inhibitors such as entacapone, A2a adenosine antagonists, cholinergic agonists, NMDA receptor antagonists and dopamine agonists.

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating, preventing, ameliorating, controlling or reducing the risk of a variety of neurological and psychiatric disorders associated with glutamate dysfunction.

Examples of disorders associated with glutamate dysfunction include: acute and chronic neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, addictive behavior, including addiction to substances (including opiates, nicotine, tobacco products, alcohol, benzodiazepines, cocaine, sedatives, hypnotics, etc.), withdrawal from such addictive substances (including substances such as opiates, nicotine, tobacco products, alcohol, benzodiazepines, cocaine, sedatives, hypnotics, etc.), obesity, psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

Anxiety disorders that can be treated or prevented by the compositions disclosed herein include generalized anxiety disorder, panic disorder, and obsessive compulsive disorder. Addictive behaviors include addiction to substances (including opiates, nicotine, tobacco products, alcohol, benzodiazepines, cocaine, sedatives, hypnotics, etc.), withdrawal from such addictive substances (including substances such as opiates, nicotine, tobacco products, alcohol, benzodiazepines, cocaine, sedatives, hypnotics, etc.) and substance tolerance.

Thus, in some aspects of the disclosed method, the disorder is dementia, delirium, amnestic disorders, age-related cognitive decline, schizophrenia, psychosis including schizophrenia, schizophreniform disorder, schizoaffective disorder, delusional disorder, brief psychotic disorder, substance-related disorder, movement disorders, epilepsy, chorea, pain, migraine, diabetes, dystonia, obesity, eating disorders, brain edema, sleep disorder, narcolepsy, anxiety, affective disorder, panic attacks, unipolar depression, bipolar disorder, psychotic depression.

Thus, provided is a method for treating or prevention schizophrenia, comprising: administering to a subject at least one disclosed compound; at least one disclosed pharmaceutical composition; and/or at least one disclosed product in a dosage and amount effective to treat the disorder in the subject. At present, the fourth edition of the Diagnostic and Statistical Manual of Mental Disorders (DSM-IV) (1994, American Psychiatric Association, Washington, D.C.), provides a diagnostic tool including schizophrenia and related disorders.

Also provided is a method for treating or prevention anxiety, comprising: administering to a subject at least one disclosed compound; at least one disclosed pharmaceutical composition; and/or at least one disclosed product in a dosage and amount effective to treat the disorder in the subject. At present, the fourth edition of the Diagnostic and Statistical Manual of Mental Disorders (DSM-IV) (1994, American Psychiatric Association, Washington, D.C.), provides a diagnostic tool including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified.

a. Treatment of a Neurological and/or Psychiatric Disorder Associated with Glutamate Dysfunction

In one aspect, the invention relates to a method for the treatment of a neurological and/or psychiatric disorder associated with glutamate dysfunction in a mammal comprising the step of administering to the mammal a therapeutically effective amount of least one compound having a structure represented by a formula:

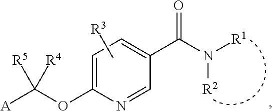

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, the mammal is a human. In a further aspect, the mammal has been diagnosed with a need for treatment of the disorder prior to the administering step. In a further aspect, the method further comprises the step of identifying a mammal in need of treatment of the disorder.

In a further aspect, the disorder is a neurological and/or psychiatric disorder associated with mGluR5 dysfunction. In a further aspect, the disorder is selected from dementia, delirium, amnestic disorders, age-related cognitive decline, schizophrenia, psychosis including schizophrenia, schizophreniform disorder, schizoaffective disorder, delusional disorder, brief psychotic disorder, substance-related disorder, movement disorders, epilepsy, chorea, pain, migraine, diabetes, dystonia, obesity, eating disorders, brain edema, sleep disorder, narcolepsy, anxiety, affective disorder, panic attacks, unipolar depression, bipolar disorder, and psychotic depression.

In a further aspect, the disorder is a disease of uncontrolled cellular proliferation. In a further aspect, the disorder is cancer, for example, breast cancer, renal cancer, gastric cancer, or colorectal cancer. In a further aspect, the disorder is selected from lymphoma, cancers of the brain, genitourinary tract cancer, lymphatic system cancer, stomach cancer, larynx cancer, lung, pancreatic cancer, breast cancer, and malignant melanoma.

b. Potentiation of Metabotropic Glutamate Receptor Activity

In one aspect, the invention relates to a method for potentiation of metabotropic glutamate receptor activity in a mammal comprising the step of administering to the mammal a therapeutically effective amount of least one compound having a structure represented by a formula:

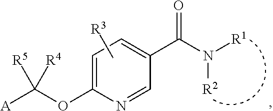

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, the mammal is a human. In a further aspect, the mammal has been diagnosed with a need for treatment of the disorder prior to the administering step. In a further aspect, the method further comprises the step of identifying a mammal in need of treatment of the disorder. In a further aspect, the metabotropic glutamate receptor is mGluR5.

c. Partial Agonism of Metabotropic Glutamate Receptor Activity

In one aspect, the invention relates to a method for partial agonism of metabotropic glutamate receptor activity in a mammal comprising the step of administering to the mammal a therapeutically effective amount of least one compound having a structure represented by a formula:

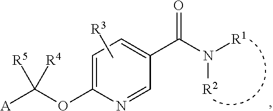

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, the mammal is a human. In a further aspect, the mammal has been diagnosed with a need for treatment of the disorder prior to the administering step. In a further aspect, the method further comprises the step of identifying a mammal in need of treatment of the disorder. In a further aspect, the metabotropic glutamate receptor is mGluR5.

d. Enhancing Cognition

In one aspect, the invention relates to a method for enhancing cognition in a mammal comprising the step of administering to the mammal an effective amount of least one compound having a structure represented by a formula:

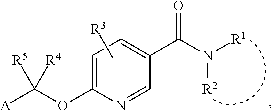

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, the mammal is a human. In a further aspect, the cognition enhancement is a statistically significant increase in Novel Object Recognition. In a further aspect, the cognition enhancement is a statistically significant increase in performance of the Wisconsin Card Sorting Test.

e. Modulating mGluR5Activity in Mammals

In one aspect, the invention relates to a method for modulating mGluR5 activity in a mammal comprising the step of administering to the mammal an effective amount of least one compound having a structure represented by a formula:

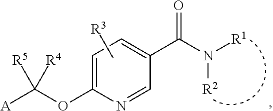

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, the mammal is a human. In a further aspect, the mammal has been diagnosed with a need for modulating mGluR5 activity prior to the administering step. In a further aspect, the mammal has been diagnosed with a need for treatment of a disorder related to mGluR5 activity prior to the administering step. In a further aspect, the method further comprises the step of identifying a mammal in need of decreasing mGluR5 activity.

In one aspect, modulating is increasing. In a further aspect, modulating is potentiation. In a further aspect, modulating is partial agonism.

In one aspect, an effective amount is a therapeutically effective amount.

In one aspect, the disorder is a neurological and/or psychiatric disorder associated with mGluR5 dysfunction. In a further aspect, the disorder is selected from dementia, delirium, amnestic disorders, age-related cognitive decline, schizophrenia, psychosis including schizophrenia, schizophreniform disorder, schizoaffective disorder, delusional disorder, brief psychotic disorder, substance-related disorder, movement disorders, epilepsy, chorea, pain, migraine, diabetes, dystonia, obesity, eating disorders, brain edema, sleep disorder, narcolepsy, anxiety, affective disorder, panic attacks, unipolar depression, bipolar disorder, and psychotic depression.

In a further aspect, the disorder is a disease of uncontrolled cellular proliferation. In a further aspect, the disorder is cancer. In a further aspect, the disorder is selected from breast cancer, renal cancer, gastric cancer, and colorectal cancer. In a further aspect, the disorder is selected from lymphoma, cancers of the brain, genitourinary tract cancer, lymphatic system cancer, stomach cancer, larynx cancer, lung, pancreatic cancer, breast cancer, and malignant melanoma.

f. Modulating mGluR5Activity in Cells

In one aspect, the invention relates to a method for modulating mGluR5 activity in at least one cell, comprising the step of contacting the at least one cell with an effective amount of least one compound having a structure represented by a formula:

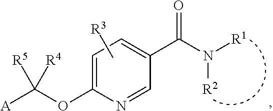

wherein - - - - - is an optional covalent bond; wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted cyclic organic residue selected from aryl, heteroaryl, cycloalkyl, heterocycloalkyl, cycloalkenyl, and heterocycloalkenyl; or a pharmaceutically acceptable salt or N-oxide thereof.

In one aspect, modulating is increasing. In a further aspect, modulating is potentiation. In a further aspect, modulating is partial agonism.

In one aspect, the cell is mammalian. In a further aspect, the cell is human. In a further aspect, the cell has been isolated from a mammal prior to the contacting step.

In a further aspect, contacting is via administration to a mammal. In a further aspect, the mammal has been diagnosed with a need for modulating mGluR5 activity prior to the administering step. In a further aspect, the mammal has been diagnosed with a need for treatment of a disorder related to mGluR5 activity prior to the administering step.

2. Manufacture of a Medicament

In one aspect, the invention relates to a method for the manufacture of a medicament for potentiation of metabotropic glutamate receptor activity in a mammal comprising combining a therapeutically effective amount of a disclosed compound or product of a disclosed method with a pharmaceutically acceptable carrier or diluent.

3. Use of Compounds

In one aspect, the invention relates to the use of a disclosed compound or a product of a disclosed method. In a further aspect, a use relates to the manufacture of a medicament for the treatment of a disorder associated with glutamate dysfunction in a mammal. In a further aspect, the disorder is a neurological and/or psychiatric disorder. In a further aspect, the disorder is a disease of uncontrolled cellular proliferation. In a further aspect, a use relates to treatment of a neurological and/or psychiatric disorder associated with glutamate dysfunction in a mammal.

In a further aspect, a use relates to potentiation of metabotropic glutamate receptor activity in a mammal. In a further aspect, a use relates to partial agonism of metabotropic glutamate receptor activity in a mammal. In a further aspect, a use relates to enhancing cognition in a mammal. In a further aspect, a use relates to modulating mGluR5 activity in a mammal. In a further aspect, a use relates to modulating mGluR5 activity in a cell.

In a further aspect, the compound has a structure represented by a formula:

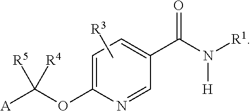

wherein R¹ is an C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide; wherein R³ is 0-1 non-hydrogen substituents independently selected from C1 to C4 alkyl, C1 to C4 haloalkyl, halide, hydroxyl, trifluoromethyl, cyano, nitro, azide, C1 to C4 alkoxy, thiol, C1 to C4 alkylsulfonyl, C1 to C4 carboxamide or C1 to C4 sulfonamide; wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; wherein A is an optionally substituted C3 to C9 cyclic organic residue selected from aryl, heteroaryl, cycloalkyl, heterocycloalkyl, cycloalkenyl, and heterocycloalkenyl; or a pharmaceutically acceptable salt or N-oxide thereof.

4. Kits

In one aspect, the invention relates to a kit comprising a disclosed compound or a product of a disclosed method and one or more of at least one agent known to increase mGluR5 activity; at least one agent known to decrease mGluR5 activity; at least one agent known to treat a neurological and/or psychiatric disorder; at least one agent known to treat a disease of uncontrolled cellular proliferation; or instructions for treating a disorder associated with glutamate dysfunction.

In a further aspect, the at least one compound or the at least one product and the at least one agent are co-formulated. In a further aspect, the at least one compound or the at least one product and the at least one agent are co-packaged.

H. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein. All ¹H NMR spectra were obtained on instrumentation at a field strength of 300 to 500 MHz.

The following exemplary compounds of the invention were synthesized. The Examples are provided herein to illustrate the invention, and should not be construed as limiting the invention in any way. The Examples are typically depicted in free base form, according to the IUPAC naming convention. However, some of the Examples were obtained or isolated in salt form.

As indicated, some of the Examples were obtained as racemic mixtures of one or more enantiomers or diastereomers. The compounds may be separated by one skilled in the art to isolate individual enantiomers. Separation can be carried out by the coupling of a racemic mixture of compounds to an enantiomerically pure compound to form a diastereomeric mixture, followed by separation of the individual diastereomers by standard methods, such as fractional crystallization or chromatography. A racemic or diastereomeric mixture of the compounds can also be separated directly by chromatographic methods using chiral stationary phases.

### 1. Methyl 6-(benzyloxy)nicotinate (1.1a).

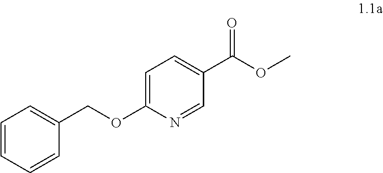

To a solution of benzyl alcohol (605 mg, 5.60 mmol) in DMF (5 mL) was added NaH (95% dry, 134 mg, 5.60 mmol) and stirred for 40 mins. Added a solution of methyl 6-bromonicotinate (1.21 g, 5.60 mmol) in DMF (5 mL) and stirred at room temperature for 18 h. The reaction was diluted with water (35 mL) and extracted with EtOAc (2×30 mL). The combined organic extracts were washed with water (2×45 mL) and brine (50 mL), dried over MgSO₄, concentrated under vacuum and purified by automated flash chromatography (silica gel) using 0 to 20% EtOAc/hexanes to afford 1.1a (890 mg, 65%) as a clear yellow oil: ¹H-NMR (400 MHz, CDCl₃) δ 8.86 (d, J=2.5 Hz, 1H), 8.16 (dd, J=8.0, 2.5 Hz, 1H), 7.51-7.32 (m, 5H), 6.84 (d, J=8.0 Hz, 1H), 5.47 (s, 2H), 3.93 (s, 3H); LC-MS (214 nm) >98%, 244.2 (M+H).

### 2. Methyl 6-(benzyloxy)-2-methylnicotinate (1.1b).

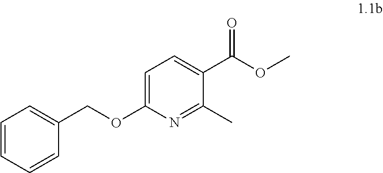

1.1b was synthesized and isolated in a similar manner to intermediate 1.1a starting from methyl 6-chloro-2-methylnicotinate (see Altman, M. et al. WO2008156726): ¹H-NMR (400 MHz, CDCl₃) δ 8.15 (d, J=8.0 Hz, 1H), 7.51-7.34 (m, 5H), 6.65 (d, J=8.0 Hz, 1H), 5.46 (s, 2H), 3.89 (s, 3H), 2.80 (s, 3H); LC-MS (214 nm) >98%, 258.2 (M+H).

### 3. Methyl 6-(3-fluorobenzyloxy)nicotinate (1.1c).

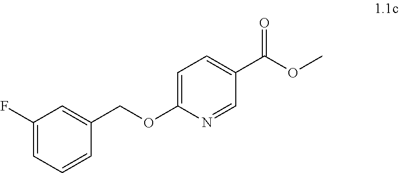

1.1c was synthesized and isolated in an similar manner to intermediate 1.1a starting from methyl 6-bromonicotinate: ¹H-NMR (400 MHz, CDCl₃) δ 8.86 (d, J=2.5 Hz, 1H), 8.20 (dd, J=8.0, 2.5 Hz, 1H), 7.41-7.33 (m, 1H), 7.21 (dd, J=17.0, 7.5 Hz, 2H), 7.03 (td, J=8.0, 2.5 Hz, 1H), 6.86 (d, J=8.0 Hz, 1H), 5.47 (s, 2H), 3.93 (s, 3H); LC-MS (214 nm) >98%, 262.2 (M+H).

### 4. Methyl 6-(4-fluorobenzyloxy)nicotinate (1.1d).

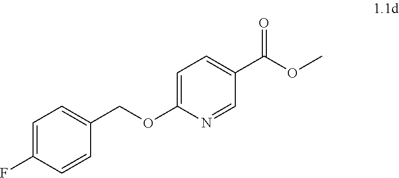

Compound 1.1d was synthesized and isolated in a similar manner to compound 1.1a: ¹H-NMR (400 MHz, CDCl₃) δ 8.63 (d, J=2.5 Hz, 1H), 8.21 (dd, J=8.0, 2.5 Hz, 1H), 7.50-7.44 (m, 2H), 7.12-7.06 (m, 2H), 6.83 (d, J=8.0 Hz, 1H), 5.32 (s, 2H), 3.94 (s, 3H); LC-MS (214 nm) >98%, 262.2 (M+H).

### 5. Methyl 6-(3,5-difluorobenzyloxy)nicotinate (1.1e).

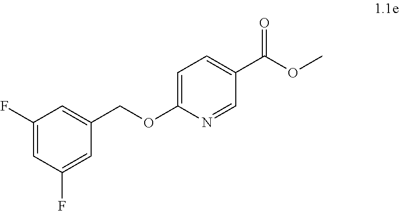

Intermediate 1.1e was prepared analogously as outlined in Scheme 1 and described for 1.1a. LC-MS (214 nm) >98%; 280.2 (M+H).

### 6. Methyl 6-(2-fluorobenzyloxy)nicotinate (1.1f).

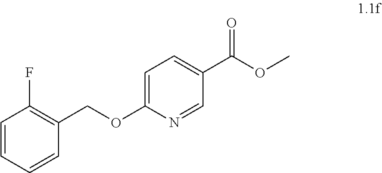

Intermediate 1.1f was prepared analogously as outlined in Scheme 1 and described for 1.1a. LC-MS (214 nm) >98%, 262.2 (M+H).

### 7. Methyl 6-(3-methylbenzyloxy)nicotinate (1.1g).

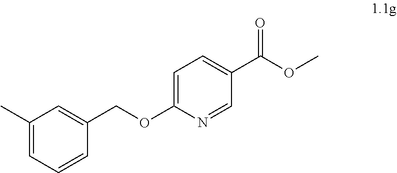

Intermediate 1.1 g was prepared analogously as outlined in Scheme 1 and described for 1.1a. LC-MS (214 nm) >98%, 258.3 (M+H).

### 8. Methyl 6-(pyridin-3-ylmethoxy)nicotinate (1.1h).

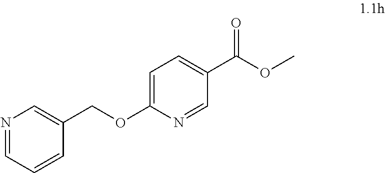

Intermediate 1.1h was prepared analogously as outlined in Scheme 1 and described for 1.1a. LC-MS (214 nm) >98%; 244.2 (M+H).

### 9. Pyridin-4-ylmethyl 6-(pyridin-4-ylmethoxy)nicotinate (1.1i).

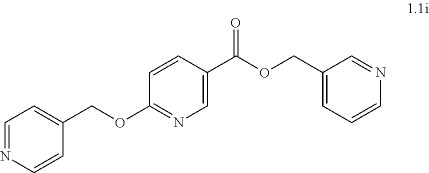

Intermediate 1.1i was prepared analogously as outlined in Scheme 1 and described for 1.1a. ¹H-NMR (400 MHz, CDCl₃) δ 8.90 (d, J=2.0 Hz, 1H), 8.66 (d, J=4.5, 2.0 Hz, 2H), 8.63 (d, J=4.5, 2.0 Hz, 2H), 8.27 (dd, J=8.0, 2.5 Hz, 1H), 7.36 (d, J=6.0 Hz, 2H), 7.34 (d, J=6.0 Hz, 2H), 6.94 (d, J=8.0 Hz, 1H), 5.52 (s, 2H), 5.40 (s, 2H); LC-MS (214 nm) >98%, 322.1 (M+H).

### 10. 6-Butoxynicotinic acid (1.1j).

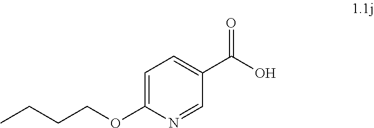

Step A. Over 0.5 h NaH (95% dry, 666 mg, 28 mmol) was added in portions to a stirred solution of neat butanol (10 mL). Methyl 6-bromonicotinate (1.5 g, 7 mmol) was added and the mixture heated in a microwave reactor for 20 min at 105° C. The reaction mixture was poured into water and extracted with EtOAc (2×35 mL). The combined extracts were washed sequentially with water and brine, then dried (Na₂SO₄), filtered and concentrated. The residue was purified using automated chromatography (silica gel) using 0-20% EtOAc in hexanes to give 1.1 g of butyl 6-butoxynicotinate (63% yield): LC-MS (214 nm) >98%, 251.9 (M+H).

Step B. Hydrolysis. A 4.0 N solution of aq. LiOH (10 mL, 40 mmol) was added to a solution of butyl 6-butoxynicotinate (1.1 g, 4.4 mmol) dissolved in MeOH (15 mL) and stirred overnight at room temperature. The reaction mixture was poured into water, acidified with HCl, and extracted with EtOAc (3×25 mL). The combined extracts were washed sequentially with water and brine, then dried (Na₂SO₄), filtered and concentrated to give intermediate 1.1j: ¹H NMR (400 MHz, CDCl₃) δ 8.92 (d, J=2.4 Hz), 8.21 (dd, J=8.0, 2.4 Hz, 1H), 6.79 (d, J=8 Hz, 1H), 4.41 (t, J=6.4 Hz, 2H), 1.80 (m, 2H), 1.50 (m, 2H), 1.00 (t, J=7.2 Hz); LC-MS (214 nm) >98%, 196.1 (M+H).

### 11. Cyclopentylmethyl 6-(cyclopentylmethoxy)nicotinate (1.1k)

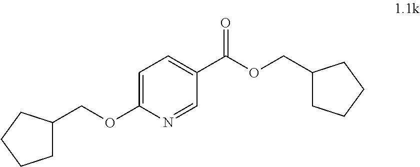

To a solution of cyclopentylmethanol (983 μL, 9.17 mmol) in DMF (10 mL) was added 95% NaH (233 mg, 9.17 mmol) and stirred for 40 mins. A DMF solution (8 mL) of tert-butyl 6-bromonicotinate (21.5 g, 8.33 mmol) was added and stirred for 18 h. The mixture was diluted with water (100 mL) and extracted with EtOAc (2×70 mL). The combined organic extracts were washed with water (2×120 mL) and brine (120 mL), dried over MgSO₄, and concentrated under reduced vacuum. The resulting residue was purified by automated flash chromatography (silica gel) using 0 to 10% EtOAc in hexanes to afford 1.1 k as a pale yellow oil: ¹H-NMR (400 MHz, CDCl₃) δ 8.83 (d, J=2.5 Hz, 1H), 8.15 (dd, J=8.0, 2.5 Hz, 1H), 6.77 (d, J=8.0 Hz, 1H), 4.31-4.19 (m, 4H), 2.43-2.31 (m, 2H), 1.90-1.79 (m, 4H), 1.73-1.55 (m, 8H), 1.44-1.32 (m, 4H); LC-MS (214 nm) >98%, 304.2 (M+H).

### 12. Methyl 6-(cyclopentylmethoxy)-2-methylnicotinate (1.1l)

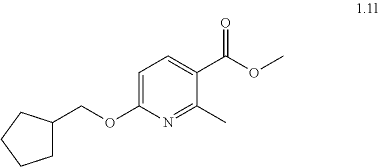

Intermediate 1.11 was synthesized in an analogous manner as 1.1 k: ¹H-NMR (400 MHz, CDCl₃) δ 8.12 (dd, J=6.0, 3.0 Hz, 1H), 6.60 (d, J=8.0 Hz, 1H), 4.21 (dd, J=18.5, 7.0 Hz, 2H), 3.99 (s, 3H), 2.78 (s, 3H), 2.44-2.27 (m, 1H), 1.90-1.78 (m, 2H), 1.73-1.57 (m, 4H), 1.44-1.29 (m, 2H); LC-MS (214 nm) >98%, 250.2 (M+H).

### 13. Methyl 6-(3-fluorobenzyloxy)-2-methylnicotinate (1.1m)

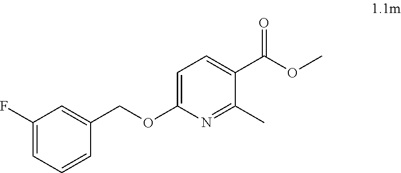

Intermediate 1.1m was synthesized in an analogous manner as 1.1 k: ¹H-NMR (400 MHz, CDCl₃) δ 8.15 (d, J=8.0 Hz, 1H), 7.34-7.31 (m, 1H), 7.28-7.18 (m, 2H), 7.11-7.05 (m, 1H), 6.68 (d, J=8.0 Hz, 1H), 5.46 (s, 2H), 3.90 (s, 3H), 2.79 (s, 3H); LC-MS (214 nm) >98%, 276.2 (M+H).

### 14. Methyl 2-methyl-6-(pyridin-4-ylmethoxy)nicotinate (1.1n)

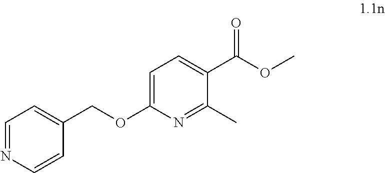

Intermediate 1.1n was synthesized in an analogous manner as 1.1 k: ¹H-NMR (400 MHz, CDCl₃) δ 8.62 (dd, J=5.0, 1.5 Hz, 2H), 8.18 (d, J=9.0 Hz, 1H), 7.37 (d, J=6.0 Hz, 2H), 6.72 (d, J=8.0 Hz, 1H), 5.49 (s, 2H), 3.90 (s, 3H), 2.76 (s, 3H); LC-MS (214 nm) >98%, 259.2 (M+H).

### 15. 6-Bromo-N-cyclohexylnicotinamide (2.2a).

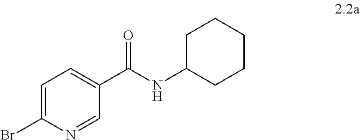

To a solution of DIPEA (1.27 g, 9.9 mmol), HATU (2.45 g, 6.4) mmol and 6-bromonicotinic acid (1.0 g, 5 mmol) in DMF (15 mL) was added cyclohexylamine (686 mg, 7.4 mmol) and the mixture was allowed to stir overnight at room temperature. The reaction mixture was poured into water and extracted with EtOAc (2×35 mL). The combined extracts were washed sequentially with water and brine, then dried (Na₂SO₄), filtered and concentrated. The residue was purified via automated flash chromatography (silica gel) using 0-35% EtOAc in hexanes to give 1.14 g of title compound (82% yield): ¹H NMR (400 MHz, CDCl₃) δ 8.69 (d, J=2.4 Hz, 1H), 7.97 (dd, J1=8 Hz, J2=2.6 Hz, 1H), 7.59 (d, J=8 Hz, 1H), 5.93 (s, br, 1H), 3.99 (m, 1H), 2.06 (m, 2H), 1.82-1.76 (m, 2H), 1.72-1.66 (m, 1H), 1.45 (m, 2H), 1.29 (m, 3H); LC-MS (214 nm) >98%, 283.2 (M+H).

### 16. 6-Bromo-N-cyclobutylnicotinamide (2.2b).

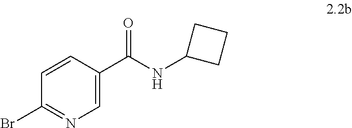

Intermediate 2.2b was prepared analogously as described for 2.2a: LC-MS (214 nm) >98%, 256.2 (M+H).

### 17. 6-Bromo-N-(3,3-dimethylbutyl)nicotinamide (2.2c)

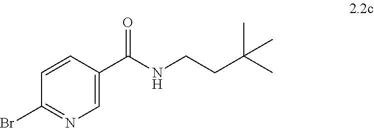

Intermediate 2.2c was prepared analogously as described for 2.2a: LC-MS (214 nm) >98%, 286.2 (M+H).

### 18. 6-(Benzyloxy)-N-(3,3-dimethylbutyl)nicotinamide

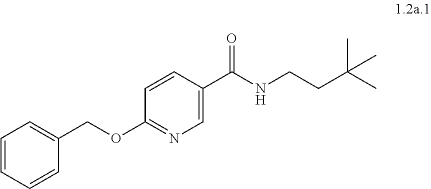

To a solution of intermediate 1.1a (600 mg, 2.47 mmol) in THF (10 ml) and MeOH (2 mL) was added a solution of LiOH (237 mg, 9.87 mmol) in water (2 mL) and stirred at room temperature for 4 h. The reaction was quenched upon addition of 1 N HCl (12 mL) and extracted with EtOAc (2×20 mL). The combined organic extracts were dried over MgSO₄ and concentrated under vacuum. The residue was dissolved in DMF (24 mL), DIPEA added (1.06 mL, 5.92 mmol) followed by HATU (1.12 g, 2.96 mmol). The solution was placed into 24 separate vials, the selected amine added (0.16 mmol) and the contents allowed to stir for 20 h. The reactions were diluted with water (5 mL) and extracted with EtOAc (2×3 mL). The combined organic extracts from each reaction were dried, concentrated and purified by mass-directed prep LC: ¹H-NMR (400 MHz, CDCl₃) δ 8.57 (d, J=2.5 Hz, 1H), 8.02 (dd, J=8.0, 2.5 Hz, 1H), 7.47 (d, J=8.0 Hz, 2H), 7.42-7.33 (m, 3H), 6.85 (d, J=8.0 Hz, 1H), 5.91 (br s, 1H), 5.45 (s, 2H), 3.53-3.45 (m, 2H), 1.58-1.52 (m, 2H), 1.00 (s, 9H); LC-MS (214 nm) >98%, 313.2 (M+H).

### 19. (R)-3-amino-2-methylbutan-2-ol

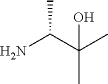

(R)-3-Amino-2-methylbutan-2-ol was prepared starting from D-alanine following one of two methods described from either Tetrahedron, 2009, 65, 3611-3614 or from WO2009075830A1.

### 20. Additional Chiral Amines

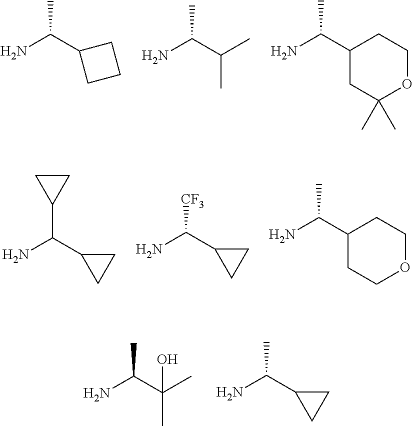

In a manner similar to the previous example, chiral amines as shown above were prepared utilizing Ellman methodology according to WO2009075830A1. Starting from the prerequisite aldehyde, Ellman reagent condensation followed. The appropriate nucleophile addition ensued followed by separation-purification and sulfinamide deprotection to give the final preferred amines according to WO2009075830A1.

### 21. (R)-6-(Benzyloxy)-N-(1-cyclohexylethyl)nicotinamide

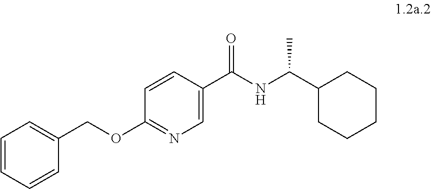

Example 1.2a.2 was prepared in a manner similar to 1.2a.1: ¹H-NMR (400 MHz, CDCl₃) δ 8.58 (d, J=2.5 Hz, 1H), 8.01 (dd, J=8.0, 2.5 Hz, 1H), 7.47-7.28 (m, 5H), 6.86 (d, J=8.0 Hz, 1H), 5.80 (br s, 1H), 5.45 (s, 2H), 4.15-4.04 (m, 1H), 1.88-1.64 (m, 5H), 1.48-1.38 (m, 1H), 1.36-1.21 (m, 2H), 1.21 (d, J=6.5 Hz, 3H), 1.20-0.98 (m, 3H); LC-MS (214 nm) >98%, 339.2 (M+H).

### 22. 6-(Benzyloxy)-N-(4-fluorophenyl)nicotinamide

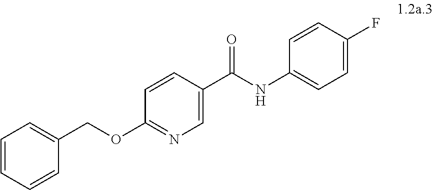

Example 1.2a.3 was prepared in a manner similar to 1.2a.1: ¹H-NMR (400 MHz, d₆-DMSO) δ 10.30 (s, 1H), 8.78 (d, J=2.5 Hz, 1H), 8.25 (dd, J=8.5, 2.5 Hz, 1H), 7.78-7.71 (m, 2H), 7.47 (d, J=8.0 Hz, 2H), 7.43-7.32 (m, 3H), 7.20 (t, J=8.0 Hz, 2H), 7.02 (d, J=8.5 Hz, 1H), 5.46 (s, 2H); LC-MS (214 nm) >98%; 323.1 (M+H).

### 23. 6-(Benzyloxy)-N-(cyclopropylmethyl)-2-methylnicotinamide

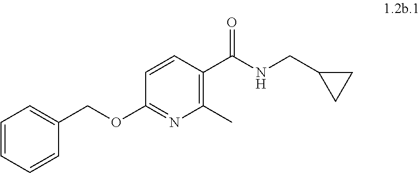

Example 1.2b.1 was prepared in a manner similar to 1.2a.1: ¹H-NMR (400 MHz, CDCl₃) δ 7.65 (d, J=8.0 Hz, 1H), 7.52-7.46 (m, 2H), 7.44-7.31 (m, 3H), 6.64 (d, J=8.0 Hz, 1H), 5.81 (br s, 1H), 5.43 (s, 2H), 3.31 (dd, J=7.0, 5.5, 2H), 2.64 (s, 3H), 1.10-1.04 (m, 1H), 0.62-0.55 (m, 2H), 0.31 (q, J=5.5 Hz, 2H); LC-MS (214 nm) >98%, 297.2 (M+H).

### 24. N-(3,3-Dimethylbutyl)-6-(3-fluorobenzyloxy)nicotinamide

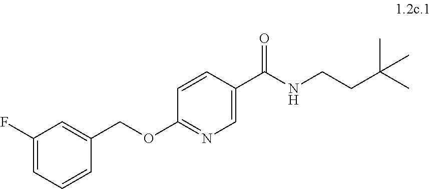

Example 1.2c.1 was prepared in a manner similar to 1.2a.1: ¹H-NMR (400 MHz, CDCl₃) δ 8.56 (d, J=2.5 Hz, 1H), 8.03 (dd, J=8.5, 2.5 Hz, 1H), 7.39-7.32 (m, 1H), 7.28-7.19 (m, 2H), 7.06-6.98 (m, 1H), 6.87 (d, J=8.5 Hz, 1H), 5.90 (s, 1H), 5.45 (s, 2H), 3.54-3.46 (m, 2H), 1.62-1.52 (m, 2H), 1.01 (s, 9H); LC-MS (214 nm) >98%, 331.0 (M+H).

### 25. N-Cyclohexyl-6-(3-fluorobenzyloxy)nicotinamide

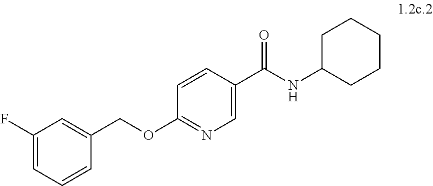

Example 1.2c.2 was prepared in a manner similar to 1.2a.1: ¹H-NMR (400 MHz, CDCl₃) δ 8.57 (d, J=2.5 Hz, 1H), 8.03 (dd, J=8.0, 2.5 Hz, 1H), 7.36-7.32 (m, 1H), 7.26-7.19 (m, 3H), 7.05-6.98 (m, 1H), 6.87 (d, J=8.0 Hz, 1H), 5.83 (br s, 1H), 5.45 (s, 2H), 4.01-3.96 (m, 1H), 2.06-2.00 (m, 2H), 1.83-1.72 (m, 3H), 1.49-1.31 (m, 2H), 1.27-1.17 (m, 3H); LC-MS (214 nM) >98%, 329.2 (M+H).

### 26. (R)—N-(1-cyclohexylethyl)-6-(pyridin-4-ylmethoxy)nicotinamide

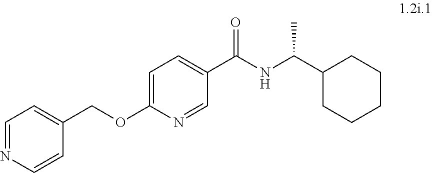

Example 1.2i.1 was prepared in a manner similar to 1.2a.1: ¹H-NMR (400 MHz, CDCl₃) δ 8.79 (d, J=7.0 Hz, 2H), 8.49 (d, J=2.5 Hz, 1H), 8.11 (dd, J=7.0, 2.5 Hz, 1H), 7.77 (d, J=6.0 Hz, 2H), 6.99 (d, J=7.0 Hz, 1H), 5.79 (s, 1H), 5.65 (s, 2H), 4.11-4.03 (m, 1H), 1.84-1.65 (m, 5H), 1.50-1.39 (m, 1H), 1.35-1.25 (m, 2H), 1.21 (d, J=7.0 Hz, 3H), 1.21-1.01 (m, 3H); LC-MS (214 nM) >98%, 340.2 (M+H).

### 27. 6-Butoxy-N-(2,4-difluorophenyl)nicotinamide

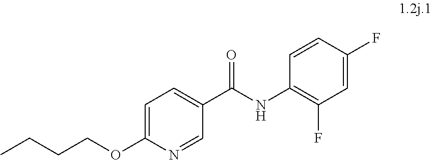

Intermediate 1.1j 6-Butoxynicotinic acid (50 mg, 0.26 mmol), 2,4-difluoroaniline (40 mg, 0.31 mmol), DIPEA (166 mg, 0.56 mmol) and HATU (136 mg, 0.36 mmol) were combined in DMF (3 mL) and stirred overnight at room temperature. The reaction mixture was poured into water and extracted with EtOAc (2×25 mL). The combined extracts were washed sequentially with water and brine, then dried (Na₂SO₄), filtered and concentrated. The residue was purified on silica gel using 0-70% EtOAc in hexanes to give 51 mg (65% yield). ¹H NMR (400 MHz, CDCl₃) δ 8.71 (d, J=2.4 Hz, 1H), 8.40-8.34 (m, 1H), 8.09 (dd, J1=8.8 Hz, J2=2.4 Hz, 1H), 7.83 (s, br, 1H), 6.97-6.91 (m, 2H), 6.84 (d, J=8.8 Hz, 1H), 4.40 (t, J=6.8 Hz, 2H), 1.84-1.77 (m, 2H), 1.54-1.48 (m, 2H), 1.00 (t, J=7.2 Hz, 3H); LC-MS (214 nm) >98%, 307.2 (M+H).

### 28. (R)—N-(1-cyclohexylethyl)-6-(3-fluorobenzyloxy)-2-methylnicotinamide

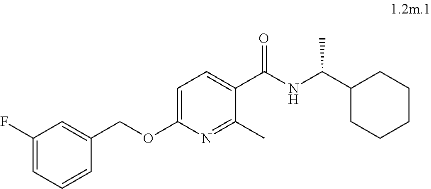

Example 1.2m.1 was prepared in a manner similar to 1.2a.1 using intermediate 1.1m: ¹H-NMR (400 MHz, CDCl₃) δ 7.61 (d, J=8.5 Hz, 1H). 7.39-7.32 (m, 1H), 7.25-7.18 (m, 2H), 7.05-6.99 (m, 1H), 6.65 (d, J=8.0 Hz, 1H), 5.49 (d, J=9.0 Hz, 1H), 5.42 (s, 2H), 4.12-4.03 (m, 1H), 2.61 (s, 3H), 1.86-1.65 (m, 5H), 1.49-1.39 (m, 1H), 1.35-1.22 (m, 2H), 1.21 (d, J=7.0 Hz, 3H), 1.21-0.99 (m, 3H); LC-MS (214 nm) >98%, 371.2 (M+H).

### 29. (R)-6-((3-fluorobenzyl)oxyl)-N-(3-hydroxy-3-methylbutan-2-yl)nicotinamide

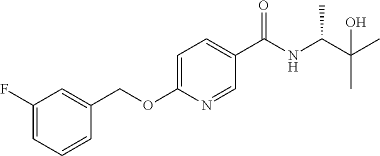

A flask charged with the TFA salt of (R)-3-amino-2-methylbutan-2-ol (659 mg, 3.0 mmol), 6-((3-fluorobenzyl)oxy)nicotinic acid (750 mg, 3.0 mmol), HATU (1.38 g, 3.6 mmol) and N,N-diisopropylethylamine (1.56 mL, 9.0 mmol) were mixed in DMF (15.0 mL) at room temperature. The reaction mixture was stirred at the same temperature for 2 h, quenched with H₂O (50.0 mL), extracted with EtOAc (30.0 mL×3). The organic layers were combined and washed with brine (60.0 mL), dried over MgSO₄, and concentrated under reduced pressure. The crude material was purified by column chromatography (EtOAc:Hexanes=10-100%), giving product (R)-6-((3-fluorobenzyl)oxy)-N-(3-hydroxy-3-methylbutan-2-yl)nicotinamide as a while solid in 67% yield. ¹H NMR (400 MHz, MeOD): δ 8.65 (d, J=2.4 Hz, 1H), 8.13 (dd, J=8.4, 2.4 Hz, 1H), 7.39 (dd, J=7.6, 6.0 Hz, 1H), 7.28 (d, J=7.6 Hz, 1H), 7.21 (d, J=9.6 Hz, 1H), 7.05 (td, J=8.4, 2.4 Hz, 1H), ), 6.95 (d, J=8.8 Hz, 1H), 5.47 (s, 2H), 4.14 (q, J=6.8 Hz 1H), 1.26-1.24 (overlapped, 9H); LCMS (ESI), single peak, m/e 333.2 ([M+1]⁺); HRMS (ESI) m/e 333.1613 ([M+1]⁺, 100%) calcd for C₁₈H₂₂N₂O₃F, 333.1614.

### 30. Tert-Butyl 2,6-dichloronicotinate (A)

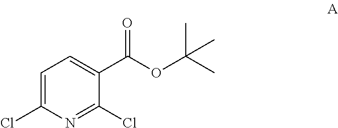

2,6-Dichloronicotinic Acid (5 g, 26 mmol) was dissolved in THF (100 mL) and treated sequentially with Boc₂O (30 mmol, 6.5 g) and DMAP (1.8 g, 15 mmol). The reaction was allowed to stir overnight, and then washed sequentially with 1M HCl (50 mL), 10% aq K₂CO₃ (2×50 mL) and brine (50 ml). The organics were dried (MgSO₄) and concentrated to a reddish oil which partially solidified on standing. A small sample was treated with 1g Silica and filtered through celite with DCM and concentrated for an NMR spectrum. The rest was used as is. ¹HNMR (400 MHz, CDCl₃) δ 8.84 (d, 1H, J=8 Hz), δ 7.68 (d, 1H, J=8 Hz), δ 1.27 (s, 9H).

### 31. Tert-Butyl 2-chloro-6-((3-fluorobenzyl)oxy)nicotinate (B)

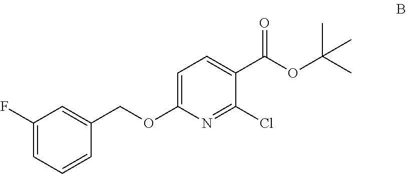

To a solution of 3-fluoro benzyl alcohol (92 mg, 0.725 mmol) in DMF (2 mL) was added NaH (95% dry, 18 mg, 0.725 mmol) and stirred for 30 mins at 0° C. The solution was cooled to −78° C. A solution of A (200 mg, 0.806 mmol) in DMF (1 mL) was cooled to 0° C. and slowly added. The reaction was allowed to warm to room temperature and stirred for 18 h. The reaction was diluted with water (10 mL) and extracted with EtOAc (2×15 mL). The combined organic extracts were washed with water (10 mL) and saturated aqueous solution of lithium chloride (10 mL), dried over Na₂SO₄, concentrated under vacuum and purified by automated flash chromatography (silica gel) using 0 to 5% EtOAc/hexanes to afford an inseparable mixture of B and a regioisomer (118 mg, 48%) as a colorless oil: LC-MS (220 nm) >98%, 338.2 (M+H).

### 32. (R)-2-chloro-N-(3,3-dimethylbutan-2-yl)-6-((3-fluorobenzyl)oxy)nicotinamide (C)

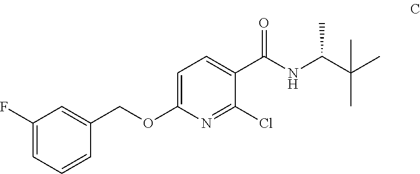

Lithium hydroxide (85 mg, 3.51 mmol) was added to a solution of B (118 mg, 0.351 mmol) in methanol (0.5 mL), THF (2 mL), and water (1 mL). The reaction was stirred at 40° C. overnight. The reaction was acidified with 1N HCl and extracted with EtOAc (2×5 mL). The combined organic extracts were washed with brine, dried over Na₂SO₄ filtered, and concentrated under vacuum. The residue was dissolved in DMF (2 mL), DIPEA (0.742 mL, 0.646 mmol) added, followed by HATU (246 mg, 0.646 mmol) and (R)-(−)-3,3-dimethyl-2-butylamine (0.066 mL, 0.497 mmol). The reaction was allowed to stir at room temperature for 20 h. The reaction was diluted with water (5 mL) and extracted with EtOAc (2×3 mL). The combined organic extracts were washed with aqueous saturated lithium chloride solution, dried over Na₂SO₄, and concentrated under vacuum. The residue was purified and the regioisomers separated on RP-HPLC using a linear gradient of acetonitrile in water (0.1% TFA) to give title compound: ¹H-NMR (400 MHz, CDCl₃) δ 8.12 (d, J=8.4 Hz, 1H), 7.38-7.3 (m, 1H), 7.16 (m, 1H), 7.06-6.97 (td, J=8.4 Hz, 2.5 Hz 1H), 6.80 (d, J=8.4 Hz, 1H), 6.49 (d, J=9.3 Hz, 1H), 5.39 (s, 2H), 4.14-4.04 (m, 1H), 1.17 (d, J=6.8 Hz, 1H), 0.98 (s, 9H); LC-MS (220 nm) >98%, 365.2 (M+H).

### 33. (R)—N-(3,3-dimethylbutan-2-yl)-6-((3-fluorobenzyl)oxy)-2-oxy)-1,2-dihydropyridine-3-carboxamide (D)

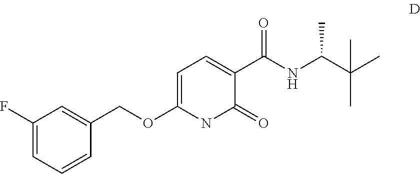

To a solution of C (26 mg, 0.0713 mmol) in 1,4-dioxane (1 mL) and water (1 mL) was added KOH (8 mg, 0.143 mmol), Pd(dba)₂ (0.82 mg, 0.0014 mmol), and tert-Butyl XPhos (2.4 mg, 0.0057 mmol). The reaction was heated with stirring at 115° C. for 60 hrs. The reaction was quenched upon addition of 2 drops of 1 N HCl and extracted with EtOAc (2×5 mL). The combined organic extracts were washed with brine (5 mL), dried over Na₂SO₄, concentrated under vacuum, and purified by RP-HPLC using a linear gradient of acetonitrile in water (0.1% TFA) to give title compound: ¹H-NMR (400 MHz, DMSO) δ 9.6 (br s, 1H), 8.3 (d, J=8.1 Hz, 1H), 7.5-7.16 (m, 4H), 6.11 (br s, 1H), 5.32 (s, 2H), 3.86 (br s, 1H), 1.05 (d, J=6.5 Hz, 1H), 0.98 (s, 9H); LC-MS (220 nm) >98%, 347.2 (M+H).

### 34. Behavior Evaluation

### In Vivo Hyperlocomotion Testing of A1

Locomotor activity was assessed as mean distance traveled (cm) in standard 16×16 photocell testing chambers measuring 43.2 cm (Length)×43.2 cm (Width)×30.5 cm (Height) (Med Associates, St. Albans, Vt.). Animals were habituated to individual activity chambers for at least 30 min prior to drug administration. Following administration of drug or vehicle, activity was recorded for a 90 minute time period. Data was expressed as the mean (±SEM) distance traveled recorded in 5 min intervals over the test period. The data was analyzed using repeated measures analysis of variance (ANOVA) followed by post-hoc testing using Dunnett's test, when appropriate. A difference was considered significant when p≦0.05.

Drugs: d-Amphetamine sulfate was obtained from Sigma (Cat#A5880-1G; St. Louis, Mo.). 10 mg of amphetamine was dissolved in 10 ml of water. Test compound was formulated in volumes of 10 mls. The appropriate amount according to the dosage was mixed into a 20% HP-β-CD solution. The solution was formulated so that animals were injected with a volume equal to 10× body weight. The mixture was then ultrahomogenized on ice for 2-3 minutes using the Dismembrator. Then the pH was checked using 0-14 EMD strips and adjusted to a pH of 6-7 if necessary. The mixture was then vortexed and stored in a warm sonication bath until time to be injected.

Doses:

1. Amphetamine 1 mg/kg, SC

2. Test compound A1, PO

3. Vehicle, pH 7, SC & IP

Animals: Male Sprague-Dawley rats weighing 225 g-275 g, between 2-3 months old (Harlan, Inc., Indianapolis, Ind.), were used. They were kept in the animal care facility certified by the American Association for the Accreditation of Laboratory Animal Care (AALAC) under a 12-hour light/dark cycle (lights on: 6 a.m.; lights off: 6 p.m.) and had free access to food and water. The experimental protocols performed during the light cycle were approved by the Institutional Animals Care and Use Committee of Vanderbilt University and conformed to the guidelines established by the National Research Council Guide for the Care and Use of Laboratory Animals.

Amphetamine-induced Hyperlocomotion: Male Harlan Sprague Dawley rats were habituated in Smart Open Field locomotor activity test chambers (Hamilton-Kinder, San Diego, Calif.) with 16×16 photobeams to automatically record locomotor activity for 30 min and then dosed with vehicle or test compound. The rats were then placed into cages. At 60 min, all rats were injected subcutaneously with 1 mg/kg amphetamine or vehicle and then monitored for an additional 60 min. Animals are monitored for a total of 120 minutes. Data are expressed as changes in ambulation defined as total number of beam breaks per 5 min periods.

Data Analysis: The data for the dose-response studies were analyzed by a between-group analysis of variance. If there was a main effect of dose, then each dose group was compared with the vehicle amphetamine group. The calculations were performed using JMP IN 8 (SAS Institute, Cary, N.C.) statistical software and graphed using SigmaPlot9 (Saugua, Mass.).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 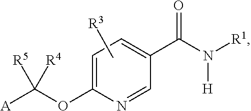
wherein R¹ is an C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide;
wherein R³ represents 0-1 substituents independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide;
wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl;
wherein A is an optionally substituted C3 to C9 cyclic organic residue selected from aryl, heteroaryl, cycloalkyl, heterocycloalkyl, cycloalkenyl, and heterocycloalkenyl;
or a pharmaceutically acceptable salt or N-oxide thereof,
wherein the compound exhibits potentiation of mGluR5 response to glutamate as an increase in response to non-maximal concentrations of glutamate in human embryonic kidney cells transfected with rat mGluR5 in the presence of the compound, compared to the response to glutamate in the absence of the compound.

2. The compound of claim 1, wherein R¹ is optionally substituted C1 to C9 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, and cyclononyl.

3. The compound of claim 1, wherein R¹ is optionally substituted aryl selected from phenyl and phenyl substituted with 1-3 groups independently selected from halide, hydroxyl, trifluoromethyl, cyano, nitro, azide, C1 to C4 alkoxy, thiol, C1 to C4 alkylsulfonyl, and C1 to C4 sulfonamide.

4. The compound of claim 1, wherein R¹ is optionally substituted heteroaryl selected from oxazolyl, isoxazolyl, pyrazolyl, furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

5. The compound of claim 1, wherein R³ is present as one non-hydrogen substituent selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

6. The compound of claim 1, wherein R³ is trifluoromethyl.

7. The compound of claim 1, having a structure represented by a formula: 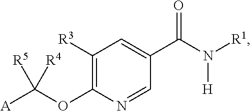
wherein R³ is selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide.

8. The compound of claim 1, having a structure represented by a formula:

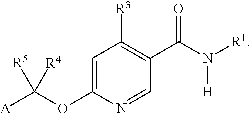

9. The compound of claim 1, having a structure represented by a formula:

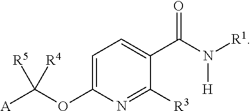

10. The compound of claim 1, having a structure represented by a formula:

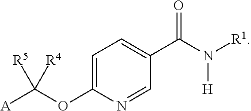

11. The compound of claim 1, wherein R⁴ and R⁵ are hydrogen.

12. The compound of claim 1, wherein R¹ has a structure represented by a formula: 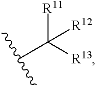
wherein R¹¹≠R¹²≠R¹³ and wherein R¹¹, R¹², and R¹³ are independently selected from hydrogen, an optionally substituted organic residue selected from alkyl, aryl, heteroaryl, cycloalkyl, heterocycloalkyl, cycloalkenyl, and heterocycloalkenyl, or two of R¹¹, R¹², and R¹³, together with the intermediate carbon, comprise an optionally substituted heterocyclic ring having from two to seven carbons, while the other of R¹¹, R¹², and R¹³ is hydrogen, an optionally substituted organic residue selected from alkyl, aryl, heteroaryl, cycloalkyl, heterocycloalkyl, cycloalkenyl, or heterocycloalkenyl,
thereby forming a stereocenter at the intermediate carbon.

13. The compound of claim 12, wherein one enantiomer of the compound has an about three-fold lower EC₅₀ for positive allosteric modulation of mGluR5 than the opposite enantiomer.

14. A method for the treatment of a neurological and/or psychiatric disorder associated with glutamate dysfunction in a mammal comprising the step of administering to the mammal a therapeutically effective amount of least one compound having a structure represented by a formula: 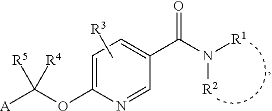
wherein - - - - - is an optional covalent bond;
wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons;
wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide;
wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl;
wherein A is an optionally substituted cyclic organic residue selected from aryl, heteroaryl, cycloalkyl, heterocycloalkyl, cycloalkenyl, and heterocycloalkenyl;
or a pharmaceutically acceptable salt or N-oxide thereof.

15. The method of claim 14, wherein the disorder is a neurological and/or psychiatric disorder associated with mGluR5 dysfunction.

16. The method of claim 14, wherein the disorder is selected from dementia, delirium, amnestic disorders, age-related cognitive decline, schizophrenia, psychosis including schizophrenia, schizophreniform disorder, schizoaffective disorder, delusional disorder, brief psychotic disorder, substance-related disorder, movement disorders, epilepsy, chorea, pain, migraine, diabetes, dystonia, obesity, eating disorders, brain edema, sleep disorder, narcolepsy, anxiety, affective disorder, panic attacks, unipolar depression, bipolar disorder, and psychotic depression.

17. The method of claim 14, wherein the compound has a structure represented by a formula: 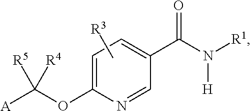
wherein R¹ is an C1 to C9 organic residue selected from alkyl, aryl, heteroaryl, cycloalkyl, heterocycloalkyl, cycloalkenyl, and heterocycloalkenyl, wherein R¹ is optionally substituted with one or more of halide, hydroxyl, trifluoromethyl, cyano, C1 to C4 alkoxy, thiol, C1 to C4 alkylsulfonyl, or C1 to C4 sulfonamide;
wherein R³ represents 0-1 substituents independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide;
wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and
wherein A is an optionally substituted C3 to C9 cyclic organic residue selected from aryl, heteroaryl, cycloalkyl, heterocycloalkyl, cycloalkenyl, and heterocycloalkenyl.

18. A method of making a compound, or pharmaceutically acceptable salt or N-oxide thereof, comprising the step of reacting a first compound having a structure represented by a formula: 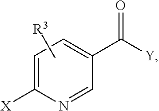
wherein X is halogen;
wherein Y is −OR⁶ or —NR¹R²;
wherein R⁶ is alkyl or aryl;
wherein R¹ is an optionally substituted C1 to C12 organic residue selected from alkyl, aryl, heteroaryl, cycloalkyl, heterocycloalkyl, cycloalkenyl, and heterocycloalkenyl and R² is hydrogen, an optionally substituted C1 to C12 organic residue selected from alkyl, aryl, heteroaryl, cycloalkyl, heterocycloalkyl, cycloalkenyl, and heterocycloalkenyl, or N, R¹, and R² together comprise an optionally substituted heterocyclic ring having from two to seven carbons; and
wherein R³ comprises three substituents independently selected from hydrogen, C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide;
with a second compound having a structure represented by a formula: 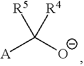
wherein R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and
wherein A is an optionally substituted cyclic organic residue selected from aryl, heteroaryl, cycloalkyl, heterocycloalkyl, cycloalkenyl, and heterocycloalkenyl,
thereby providing a compound having a structure represented by a formula:

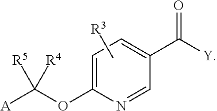

19. The method of claim 18, wherein reacting is a nucleophilic substitution reaction in the presence of sodium hydride.

20. The method of claim 18, wherein the compound provided has a structure represented by a formula: [chemical structure]
